(19) United States
(12) Patent Application Publication
(10) Pub. No.: US 2021/0293825 A1
(43) Pub. Date: Sep. 23, 2021

(54) METHODS FOR PRECISION THERAPEUTIC TARGETING OF HUMAN CANCER CELL MOTILITY AND KITS THEREOF

(71) Applicant: UNITED STATES GOVERNMENT AS REPRESENTED BY THE DEPARTMENT OF VETERANS AFFAIRS, Washington, DC (US)

(72) Inventors: Raymond Bergan, Portland, OR (US); Ryan Gordon, Portland, OR (US)

(21) Appl. No.: 17/262,556

(22) PCT Filed: Feb. 7, 2019
(86) PCT No.: PCT/US2019/017006

§ 371 Date: Jan. 22, 2021

Related U.S. Application Data
(60) Provisional application No. 62/628,243, filed on Feb. 8, 2018.

(51) Int. Cl. G01N 33/68

(52) U.S. Cl. CPC G01N 33/6845; G01N 2440/14; G01N 2500/20; G01N 2500/02

(57) ABSTRACT

Disclosed are methods for identifying an agent of interest that alters binding or activity of a client protein to a chaperone and kits thereof.

### CROSS REFERENCE TO RELATED APPLICATIONS

This application claims the benefit of the filing date of U.S. Provisional Application No. 62/628,243, filed on Feb. 8, 2018. The content of this earlier filed application is hereby incorporated by reference herein in its entirety.

### STATEMENT REGARDING FEDERALLY SPONSORED RESEARCH

This invention was made with government support under grant number IBX002842A awarded by the U.S. Department of Veterans Affairs and grant number P30 CA069533 awarded by the National Institutes of Health. The government has certain rights in the invention.

### INCORPORATION OF THE SEQUENCE LISTING

The present application contains a sequence listing that was submitted in ASCII format via EFS-Web concurrent with the filing of the application, containing the file name “37759_0083P1_Sequence_Listing.txt” which is 8,192 bytes in size, created on Jan. 25, 2019, an is herein incorporated by reference in its entirety.

### BACKGROUND

Chaperone proteins play important regulatory roles in the cell, affecting a wide range of biological processes. They mediate their effects by inducing changes on their client proteins. There are hundreds of client proteins in the cell. The largest category of client proteins are protein kinases. There are over 400 known protein kinases, and most of them are considered to be client proteins. General inhibitors of chaperone proteins, such as HSP90, directly bind to HSP90 and inhibit its chaperone activity. In this manner, they broadly inhibit the function of many client proteins. Such an effect is highly toxic to cells, to animals and to humans, and thereby has precluded their use as therapeutics for human diseases. It has been difficult to identify chemicals, i.e., drug candidates that selectively inhibit chaperone protein-mediated effects on client proteins. A major reason for this relates to the highly complex nature of the mechanism of action of how chaperone proteins work.

### SUMMARY

Other features and advantages of the present compositions and methods are illustrated in the description below, the drawings, and the claims.

Disclosed herein are methods for identifying one or more agents of interest that alter binding or activity of a client protein to a chaperone-co-chaperone complex, the methods comprising: a) forming a cell-free chaperone-co-chaperone complex in vitro with an isolated chaperone protein and a co-chaperone protein; b) incubating the chaperone-co-chaperone complex with a client protein in the presence or absence of the one or more agents of interest; c) assaying the binding of the client protein to the chaperone-co-chaperone complex or activity of the client protein in step (b); and d) determining whether the one or more agents of interest alter the binding or activity of the client protein in step (c), thereby identifying the one or more agents of interest that alter binding or activity of the client protein to the chaperone-co-chaperone complex.

Disclosed herein are methods for identifying one or more agents of interest that alters binding or activity of a client protein to a chaperone-co-chaperone complex, wherein the chaperone-co-chaperone complex is HSP90β/CDC37, the methods comprising: a) forming a cell-free chaperone-co-chaperone complex in vitro with an isolated chaperone protein and a co-chaperone protein; b) incubating HSP90β/CDC37 with a client protein in the presence or absence of the one or more agents of interest; c) assaying the binding of the client protein to HSP90β/CDC37 or activity of the client protein in step (b); and d) determining whether the one or more agents of interest alter the binding or activity of the client protein in step (c), thereby identifying the one or more agents of interest that alter binding or activity of the client protein to HSP90β/CDC37.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1a-f show that KBU2046 selectively inhibits cell motility. FIG. 1a is a schematic flow of probe synthesis and development strategy. FIG. 1b shows human prostate metastatic cells (PC3, PC3-M), and HPV transformed normal (1532NPTX, 1542NPTX) and primary cancer (1532CPTX, 1542CPTX) cells, that were treated with 10 μM genistein (G), KBU2046 (46) or vehicle (CO), and after 3 days, cell invasion was measured. Values are mean±SEM of a single experiment in replicates of N=4, with similar findings in multiple separate experiments (also N=4). FIG. 1c shows single cell migration that was measured after treatment for 3 days with 10 μM KBU2046 or vehicle (control). Values are mean±SEM of a single experiment in replicates of N=24, with similar findings in a separate experiment (also N=24). *Denotes Student's t test P value <0.05, compared to controls. FIG. 1d shows that the concentrations at which KBU2046 or genistein inhibited cell growth after 3 days by 20% (IC₂₀) and 50% (IC₅₀), as is the percentage of growth inhibition at 50 μM. Values are mean±SEM from N>2 separate experiments, each in replicates of N=3. FIG. 1e shows the results of a human cord blood hematopoietic stem cell colony formation assay. Values are the mean±SD number of total, CFU-GM, CFU-GEMM or BFU-E colonies at 14 days after treatment with KBU2046, from a single experiment in replicates of N=2. FIG. 1f shows the induction of estrogen-responsive genes. Values are the mean±SD of a single experiment, with similar results seen in a separate experiment, both in replicates of N=2.

FIGS. 2a-d show that KBU2046 inhibits cancer metastasis and prolongs life. Cohorts of N=12 athymic mice bearing human PCa PC3-M cell orthotopic implants (a), or of N=5 non-tumor bearing athymic mice (b), were treated with KBU2046 incorporated into chow, and resultant lung metastasis (a) or plasma KBU2046 concentration (b) measured. Values are the mean±SEM. The relationship between dose and metastasis was evaluated by two-sided ANOVA (a). FIG. 2c a comprehensive characterization of KBU2046 pharmacokinetics. CD1 mice were dosed with 100 mg KBU2046/kg via oral gavage or intravenous injection (iv), and blood collected at the indicated time points (data from mice dosed at 25 mg/kg are in FIG. 12, and corroborate 100 mg/kg findings). For each route and time point, N=3 mice were sampled. Individual data points are the resultant plasma concentrations from individual mice, and are the mean of N=2 measurements. The dotted horizontal line denotes a concentration of 24 nM, which was the concentration of KBU2046 measured in the blood of mice whose metastasis were suppressed by 92% (a-b). FIG. 2d shows prolongation of survival in BCa bearing mice. Mice were orthotopically implanted with human breast cancer LM2-4H2N cells, the resultant primary tumors resected, and adjuvant treatment begun with KBU2046 by daily oral gavage five times/week. The survival of N=6 mice receiving vehicle was compared to that of N=6 mice receiving 25 mg/kg KBU2046 by the log rank (Mantel-Cox) test.

FIGS. 3a-e show that KBU2046 inhibits bone destruction. FIG. 3a shows the treatment schema. Athymic mice were given intracardiac (IC) injections of PC3-luc cells on day 0 under ultrasound guidance, and underwent weekly IVIS imaging starting seven days post injection. Cohorts of N=20 control, N=20 Pre (treatment from 3 days prior to IC injection through end of experiment), N=10 Pre7Stop (treatment from 3 days prior to IC injection through 7 days post IC injection) and N=10 Post treatment (treatment from 3 days post-IC injection through end of experiment) mice were dosed with 80 mg/kg KBU2046 daily by oral gavage and mock treated with vehicle all other times. FIG. 3c depicts whole body and FIG. 3b depicts mandible flux as determined from weekly IVIS imaging. FIG. 3d shows the results at week 4 post injection (i.e., at the end of the experiment), CT scans were performed on control, Pre and Post treatment cohorts, and mandibular destruction quantified. FIG. 3e shows representative images of Pre and control mice. Arrows denote areas of bone destruction in controls, and corresponding areas in the Pre mouse. Student's t test (b-c) and Fisher's exact test (d) P values between the denoted cohorts are shown.

FIGS. 4a-d show that KBU2046 decreases phosphorylation of HSP90β. FIG. 4a shows that probing for KBU2046-induced changes in protein phosphorylation. PC3-M or PC3 cells were pre-treated with 10 μM KBU2046 for 3 days, then with ±TGFβ and the resultant cell lysate probed for changes in protein phosphorylation with the KinomeView® assay. The depicted Western blot utilizes KinomeView® phospho-motif antibody, BL4176; the blue arrow denotes an 83 kDa band whose phosphorylation is inhibited by KBU2046 (see FIG. 17 for complete KinomeView® assay screening data). FIG. 4b shows the proteomic analysis. PC3 cells were pre-treated with KBU2046 or vehicle, then with TGFβ, proteins from the resultant cell lysate were immunoprecipitated with BL4176, and HSP90β was identified by LC-MS/MS analysis (SEQ ID NO: 1); see FIG. 19 for expanded proteomic assay data). The phospho-motif recognized by the antibody is underlined; S*- denotes Ser226, whose phosphorylation is decreased by KBU2046. FIGS. 4c and 4d show that the phospho-mimetic changes in HSP90β Ser²²⁶ structure regulate human PCa cell invasion and KBU2046 efficacy. PC3-M cells were transfected with S226A-, S226D-, or WT-HSP90β, or empty vector (VC), treated with KBU2046 or vehicle, and cell invasion measured. Values are the mean±SEM of a representative experiment of multiple experiments (all in replicates of N=3); *denotes t-test P value <0.05 between bracketed conditions, or compared to VC.

FIGS. 5a-d show that KBU2046 stabilizes CDC37/HSP90β heterocomplexes. FIG. 5a shows that KBU2046 stabilizes HSP90β/CDC37 heterocomplexes in a DARTS assay. Equimolar amounts of HSP90β and CDC37 protein were pre-incubated with KBU2046, and resultant thermolysin reaction products were detected by silver stain following SDS-PAGE. The mean value (from N=3 independent experiments) of protein bands indicated by arrows is displayed below each lane, and are expressed as the percentage of untreated control. ANOVA P values for changes in band intensity with concentration are displayed. FIG. 5b shows the In-silico model of CDC37 (purple) and HSP90β (grey) depicting KBU2046 hydrogen bonding with Gln119 of HSP90β. FIG. 5c shows the lipophilic potential surface of the computed ligand binding pocket of the CDC37/HSP90β model with KBU2046 bound (color code: brown—hydrophobic; green—hydrophilic). FIG. 5d shows the potential surface of the whole CDC37/HSP90β dimer (color code: green—HSP90β; grey—CDC37; cyan—Arg167 from CDC37 bisecting the larger pocket and creating a new cleft into which KBU2046 binds).

FIGS. 6a-g shows KBU2046-mediated changes in the signature of client proteins bound to the HSP90β/CDC37 heterocomplex mediate effects upon cell motility. FIG. 6a shows the results of the LUMIER assay. HEK293T cells were transfected with 1 of 420 different protein kinases, treated with 10 μM KBU2046 (N=5 replicates) or vehicle control (N=5) for three days, and LUMIER assays performed. The N=17 kinases that gave significant findings (Student's t-test <0.05) in the same direction in each of two separate experiments are depicted. Each separate treatment and kinase condition in each of two separate experiments was conducted in replicates of N=5. FIG. 6b shows that the experiment was then repeated for these 17 kinases in the presence of TGFβ treatment, and those demonstrating significant differences (t-test <0.05) in the same direction as in (a) are denoted by *. FIGS. 6c and 6d show the results of the wound healing assay. PC3 cells were transfected with siRNA targeting RAF1 (si-Raf1) or non-targeting siRNA (si-control), treated with KBU2046 or vehicle as above, and RAF1 protein measured by Western blot (c) and effects upon wound healing measured (d). FIG. 6e shows the inhibition of RAF1. Purified recombinant HSP90β, CDC37, and RAF1 were combined with KBU2046, as indicated, incubated in an in vitro kinase assay for the indicated times, and Western blot for RAF1-Ser³³⁸ phosphorylation performed. FIGS. 6f and 6g show the effect on HSP90β/CDC37 heterocomplex formation and function in vitro. Purified recombinant HSP90β, CDC37, RAF1, SGK3 or MAP3K6 were combined and treated with KBU2046 or vehicle control, as indicated, incubated in an in vitro kinase assay for the indicated times, and Western blot performed, as denoted. Experiments were repeated at separate times at least once, with similar results.

FIG. 7 shows that KBU2046 inhibits Raf1 activation in human prostate cancer cells. PC3 and PC3-M human prostate cancer cells were treated with 10 μM KBU2046 for the indicated lengths of time; control cells (C) were treated with vehicle. Levels of RAF1-Ser³³⁸ phosphorylation (pRaf1), total RAF1, and GAPDH proteins were then measured by Western blot.

FIGS. 8a-d shows the synthesis of KBU2046. Following the synthetic strategy outlined in FIG. 1a, 4′,5,7-trihydroxyisoflavone was used as the chemical scaffold. FIG. 8a shows synthetic round #1. As this scaffold had anti-invasion efficacy, it was first evaluated which of its chemical fragments were important for activity by synthesizing a set of compounds lacking individual functional groups, and assessing their effects upon cell invasion and cell growth inhibition. Key informative findings include but are not limited to: the ring C4′-hydroxyl group is important for activity (compare compounds 1 and 2) and removal of the C7-hydroxyl group (which mediates binding to the ER) does not affect activity (compare compounds 2 and 8). Other relevant findings include but are not limited to: movement of the C4′-hydroxyl is associated with retention of activity (compare compounds 2 and 5), and it is possible to achieve growth inhibition while having minimal impact upon invasion; consider compounds 1, 3, 4, and 6. Note, demethylation within the cell cannot be predicted. Therefore, the loss of function was considered to be informative for methylated compounds. For example, consider compound 7, where methylation of the C4′-hydroxyl group leads to loss of invasion (compare to compound 8). This adds further evidence of the importance of the C4′-hydroxyl for activity. In contrast, while the C7- and C4′-hydroxyl groups of compound 16 are methylated, it retains anti-invasion activity, indicating that demethylation within the cell could possibly influence the results. For cell invasion, PC3-M cells were treated with 10 μM compound for a total of 3 days, and cell invasion assays were run at the end of the 3 day period, in the presence of compound. Values are the mean±SEM of three separate experiments, each run in replicates of N=3. Three day MTT cell growth inhibition assays were performed with PC3-M cells. Values are the mean±SEM of a single experiment, in replicates of N=4, repeated at least once (also N=4). FIG. 8b shows synthetic round #2. The initial structure-activity relationship (SAR) data informed the second round of compound synthesis. Key biological findings include but are not limited to: it is possible to retain anti-invasion efficacy while having minimal effect upon cell growth inhibition (compound 22). Additionally, reduction of the C2-C3 double bond does not confer loss of activity (compound 22) and appears to reduce off-target cell toxicity. Other findings include but are not limited to: moving the C4 carbonyl group to generate the coumarin core confers loss of activity (consider compounds 23, 24 and 25). FIG. 8c shows synthetic round #3. FIG. 8d shows synthetic round #4. Key findings include but are not limited to: a new chemical entity was identified, now termed KBU2046 (compound 46), with anti-invasive efficacy at least equal to that of the starting compound, 4′,5,7-trihydroxyisoflavone, but that has no growth inhibitory effects. Compared to 4′,5,7-trihydroxyisoflavone, KBU2046 is non-planar, lacks hydroxyl groups, and particularly those that mediate ER binding, is halogen-substituted, and has a distinctly different biological profile. These characteristics place KBU2046 in a chemically distinct class, compared to the starting compound. Further, KBU2046 possesses novel biological characteristics; described herein.

FIG. 9 shows KBU2046 has minimal-to-no cell toxicity in the NCI 60 cell line panel. KBU2046 was submitted to the Developmental Therapeutics Program (DTP) of the US National Cancer Institute (NCI), underwent initial screening across the NCI 60 cell line panel per DTP protocol (Shoemaker, R. H., Nat Rev Cancer 6, 813-23 (2006)) and the resultant COMPARE diagram is depicted herein. Based upon its lack of cell toxicity, NCI did not select KBU2046 to go on to multi-dose testing.

FIG. 10 shows that KBU2046 does not activate the estrogen receptor (ER). Values are the mean±SD of a single experiment, with similar results seen in a separate repeat experiment, both in replicates of N=2.

FIG. 11 shows the chemical properties of KBU2046 that favor its ability to reach the cellular target when delivered systemically.

FIGS. 12a-b shows the results of the pharmacokinetic (PK) analysis of KBU2046. FIG. 12a shows concentration versus time plot. Individual data points are the resultant plasma concentrations from individual mice, are the mean of N=2 measurements, and are expressed as ng/ml. The 100 mg dosing data was re-plotted and expressed as nM, and constitutes FIG. 2c. FIG. 12b shows the resultant pharmacokinetic parameters.

FIG. 13 shows that KBU2046 does not inhibit primary tumor cell growth. Data are the mean±SEM tumor weight of mice treated in FIG. 2a.

FIG. 14 shows that KBU2046 treatment is not associated with systemic off-target effects.

FIGS. 15a-c shows intra-cardiac injection of PC3-luc cells. FIG. 15a shows PC3-luc cells were injected under ultrasound guidance into the left ventricle. Depicted are snapshots of real time ultrasound images of a mouse undergoing intra-cardiac (IC) injection. The mouse is positioned with the head to the right and the sternum on top. Red arrow: left ventricle; green arrow: the needle positioned within the left ventricle; yellow arrow: injectate containing PC3-luc cells exiting the ventricle through the aorta. FIG. 15b confirms that the injection was successful. Mice under IVIS imaging 30 minutes post IC injection. Successful injections are characterized by a relatively uniform distribution throughout the body, consistent with cells being injected into and distributed by the vasculature. Unsuccessful injections lack this pattern and are characterized by a local collection of cells in the thoracic cavity. FIG. 15c shows representative IVIS images. Depicted are IVIS images from a control (non-treated) mouse, and from one treated with KBU2046 (i.e., treatment began 3 days prior to IC injection).

FIGS. 16a-e show that KBU2046 does not inhibit the MKK4 pathway. FIG. 16a is a depiction of established MKK4 pathway regulating human PCa cell metastasis. FIG. 16b shows that KBU2046 does not bind to MKK4, as measured by fluorescence-based thermal shift assay. Values are the mean±SD (of N=2 replicates) increase in melting temperature (ΔTm) of purified recombinant MKK4 induced by the indicated concentrations of KBU2046 or genistein. FIG. 16c shows that KBU2046 does not inhibit MKK4 in an in vitro kinase assay. The indicated concentrations of KBU2046 were added to recombinant activated MKK4, and its ability to phosphorylate kinase dead p38a MAPK (K53A) was measured by Western blot for total (p38 MAPK) and phosphorylated (pp38 MAPK) forms of p38 MAPK. FIGS. 16d and 16e show that KBU2046 does not inhibit downstream phosphorylation of p38 MAPK or of HSP27 in cells. PC3-M cells were pre-treated for 24 hours with 50 μM KBU2046 or genistein, as indicated, then with TGFβ, and Western blot performed. These findings are from a single experiment, with similar findings in identical separate experiments, repeated at least once.

FIGS. 17a-c show proteomic analysis of the effects of KBU2046 on the kinome and screening for effects on the kinome. FIG. 17a shows the identification of an 83 kDa band of interest (red arrows). This constitutes the change that was repeatable across two experiments, and it was observed in PC3 and PC3-M cells, as well as in tumor tissue. FIG. 17b shows bands of initial potential interest that did not repeat (blue arrows). FIG. 17b shows the other Western blots of phospho-motif antibodies that were evaluated on initial screen. NI=tumor not informative; this denotes a tumor sample that yielded an abnormal coomassie blue staining pattern. Data from this sample was therefore not considered further.

FIG. 18 shows that KBU2046 retains efficacy even under conditions of TGFβ-stimulated increases in cell invasion. The invasion of PC3-M cells pretreated with KBU2046 or vehicle control (CO) and then with ±TGFβ was measured. Data are the mean±SEM of a single experiment; similar findings were observed in a replicate experiment (both experiments were in replicates of N=4).

FIGS. 19a-b show the results of the identification of the 83 kDa band using a proteomic approach. FIG. 19a shows PC3 cells that were pre-treated with 10 μM KBU2046 or vehicle (control) for 3 days, then treated with TGFβ for 1 hr, and the resultant cell lysates were subjected to immunoprecipitation with BL4176 (Kinoview® phospho-motif antibody). SEQ ID NOs: 2-20 are listed (in order from top to bottom under the heading “Peptide”. FIG. 19b shows the evaluation of HSP90β levels. In order to assess whether phosphorylation changes detected in FIG. 17a were not due to changes in HSP90β protein, total HSP90β protein levels were measured by Western blot, after treatment of PC3 and PC3-M cells as described in FIG. 17a.

FIGS. 20a-d show that changes in HSP90β Ser²²⁶ structure as well as HSP90β expression regulate PCa cell invasion and KBU2046 efficacy. FIG. 20a shows that levels of HSP90β expression after transfection of cells. In FIGS. 4c and 4d, PC3-M cells were transfected with S226A, S226D-, or WT-HSP90β, or empty vector (VC) and resultant effects upon cell invasion and KBU2046 efficacy measured. Depicted herein are associated Western blots probing for HSP90β, FLAG (transfected HSP90β was FLAG-tagged) and GAPDH. FIGS. 20b-d show that the knockdown of HSP90β decreases cell invasion and abrogates KBU2046 efficacy. PC3-M cells were transfected with siRNA to HSP90β (siHSP90b) or non-targeting siRNA (siCO). FIG. 20b shows that the level of HSP90β (HSP90b) and HSP90α (HSP90α) transcript levels were measured by qRT/PCR, and expressed relative to that of GAPDH. FIG. 20c shows that the level of HSP90β protein expression was measured by Western blot. FIG. 20d shows cells were treated with KBU2046 (46) or vehicle control, and cell invasion was measured. Values are the mean±SEM of a representative experiment of multiple experiments (all in replicates of N=3). *denotes t-test P value </=0.05 compared to siCO.

FIG. 21 shows that KBU2046 does not bind HSP90β or CDC37. Studies used purified recombinant HSP90β or CDC37. Fluorescent thermal shift (Krishna, S. N. et al., PLoS One 8, e81504 (2013)), isothermal titration calorimetry (Chavez, J. D., et al., Mol Cell Proteomics 12, 1451-67 (2013)), and biolayer interferometry assays (Makowska-Grzyska, M. et al., Biochemistry 51, 6148-63 (2012)), were performed, and failed to provide evidence of KBU2046 binding to either HSP90β or CDC37. For DARTS assay, HSP90β or CDC37 were individually pre-incubated with KBU2046, thermolysin added, and reaction products were separated by SDS PAGE and visualized by silver stain (depicted above). NTco—no thermolysin control. However, when the DARTS assay was conducted with both HSP90β and CDC37 present, KBU2046 protected both proteins from degradation in a concentration-dependent manner (see, FIG. 5a).

FIG. 22-d show that KBU2046 binds to intact cells, but not to isolated proteins. FIG. 22a shows that the chemical structure of KBU2046 linked to biotin (KBU2046-biotin) as synthesized. FIG. 22b shows that KBU2046-biotin is biologically active. PC3-M cells were pre-treated with 10 μM KBU2046 or with KBU2046-biotin for three days, and single cell motility assays conducted. Data are the mean±SEM of a single experiment of N≥24 cells; * denotes Student's 2-sided t-test p-value </=0.05 compared to control. FIG. 22c shows that KBU2046-biotin labels permeablized cells in a manner that can be competed off. PC3-M cells were labeled with 1 μM KBU2046-biotin+/−10 μM free KBU2046, followed by detection with FITC-streptavidin, and visualization by fluorescent microscopy (with equal exposure times). FIG. 22d shows protein array hybridization. KBU2046-biotin was hybridized to ProtoArray® Human Protein Microarray's at 0.5 and 10 μM with and without 10 fold excess free KBU2046.

FIGS. 23a-c show the construction of a structural model of HSP90β, CDC37 and KBU2046 interaction. FIG. 23a shows the HSP90 (magenta) nucleotide binding site surface (A, yellow) shown with bound inhibitor (Wright, L. et al., Chem Biol 11, 775-85 (2004)). When complexed with CDC37 (gray), a large cleft is formed at the interface (b). The CDC37 Arg167 residue dissects the cleft into two distinct sub-pockets (c). The nucleotide binding surface (C, yellow) is preserved, but a new sub-pocket (cyan) is formed. KBU2046 is shown docked into the newly formed site (C, wheat).

FIGS. 24a-d show that KBU2046-mediated changes in the signature of client proteins bound to HSP90β/CDC37 mediates effects upon cell motility. FIG. 24 a shows the results of the LUMIER assay (a). FIGS. 24b-d show the results of the wound healing assay. PC3 or PC3M cells were treated+/−KBU2046 and with siRNA to the denoted gene, or with non-targeting control siRNA (NT), and effects upon transcript (b) or protein expression (c) measured by qRT/PCR or Western blot, respectively. For qRT/PCR, values are expressed as the target gene/GAPDH ratio, normalized to that of NT/−KBU2046 cells. Under the same conditions, wound healing assays were performed (d).

FIG. 25 shows that KBU2046 affects client protein function and interaction with HSP90β/CDC37 heterocomplexes in vitro. RAF1, CDC37 and HSP90β proteins were added, as denoted, in a RAF1 in vitro kinase assay, and autophosphorylation of RAF1 detected by Western blot with phospho-CK2 antibody. Non-specific binding to HSP90β and CDC37 proteins provides a measure of their presence and equal amounts for loading controls.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a derivative is disclosed and discussed and a number of modifications that can be made to a number of molecules are discussed, each and every combination and permutation of derivative and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D.

Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Other than in the operating examples, or where otherwise indicated, all numbers expressing quantities of ingredients or reaction conditions used herein should be understood as modified in all instances by the term “about.” The term “about” when used in connection with percentages can mean a range of +1-10%.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Thus, for example, reference to “a therapeutic” includes a plurality of such therapeutics; reference to “the therapeutic” is a reference to one or more therapeutics known to those skilled in the art, and so forth.

The use of the term “containing,” as well as other forms, such as “contains” and “contained,” is not limiting. Also, terms such as “element” or “component” encompass both elements or components comprising one unit and elements or components comprising more than one unit unless specifically stated otherwise.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety.

The term “therapeutic” refers to a composition that treats a disease.

As used herein, the term “subject” or “patient” refers to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition. In some instances, a therapeutically effective amount is an amount of a therapeutic that provides a therapeutic benefit to an individual.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer or cancer metastasis.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75-100 percent as compared to native or control levels.

“Modulate”, “modulating” and “modulation” as used herein mean a change in activity or function or number. The change may be an increase or a decrease, an enhancement or an inhibition of the activity, function, or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100 percent, or more, or any amount of promotion in between compared to native or control levels. In an aspect, the increase or promotion is 10-20, 20-30, 30-40, 40-50, 50-60, 60-70, 70-80, 80-90, or 90-100 percent as compared to native or control levels. In an aspect, the increase or promotion is 0-25, 25-50, 50-75, or 75-100 percent, or more, such as 200, 300, 500, or 1000 percent more as compared to native or control levels. In an aspect, the increase or promotion can be greater than 100 percent as compared to native or control levels, such as 100, 150, 200, 250, 300, 350, 400, 450, 500 percent or more as compared to the native or control levels.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample).

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the term “agent of interest” refers to a “test compound” or a “drug candidate compound”. As such, these compounds can comprise organic or inorganic compounds, derived synthetically or from natural sources. Examples of said compounds include but are not limited to peptide, polypeptide, protein, nucleic acid, antibodies, oligomer, polymer or small molecule, and the like.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the phrase “an agent of interest that alters binding or activity” can mean a compound that inhibits or stimulates or can act on another protein which can inhibit or stimulate the protein-protein interaction of a complex two proteins.

As used herein, the term “client protein” refers to a protein that can be manipulated or processed, for example, folding by one or more chaperone proteins. Examples of client proteins include but are not limited to kinases.

As used herein, the term “chaperone complex” or “chaperone-co-chaperone complex” or “heterocomplex” refers to a group of two or more associated polypeptide chains or proteins. In an aspect, a chaperone-co-chaperone complex can refer to Hsp90b-Cdc37. Proteins or polypeptide chains (e.g., chaperone or chaperone protein) in a chaperone complex or chaperone-co-chaperone complex can be linked by non-covalent protein-protein interactions. A “chaperone” or “chaperone protein” are also known as “molecular chaperones”. A “chaperone” or “chaperone protein” or “molecular chaperone” is a protein that assists the covalent folding or unfolding and the assembly or disassembly of other macromolecular structures.

Disclosed herein are compositions and methods that serve to overcome the problem of identifying drug candidates that selectively inhibit chaperone protein-mediated effects on client proteins. Described herein are methods and assays that permit the biological effect of chaperone proteins on client proteins to be measured. Further, the disclosed assays and methods can be used to measure the effect chemicals (or drug candidates) may have on how chaperone proteins affect client proteins. As such, the methods and assays disclosed herein can be used to screen large sets of existing and new chemicals for their ability to affect specific individual, or sets of, client proteins. Further, these “effects” can in turn relate to the regulation of a wide range of biological processes. In this manner, the disclosed methods and assays can be used to efficiently screen for drugs that selectively act on a wide range of biological processes.

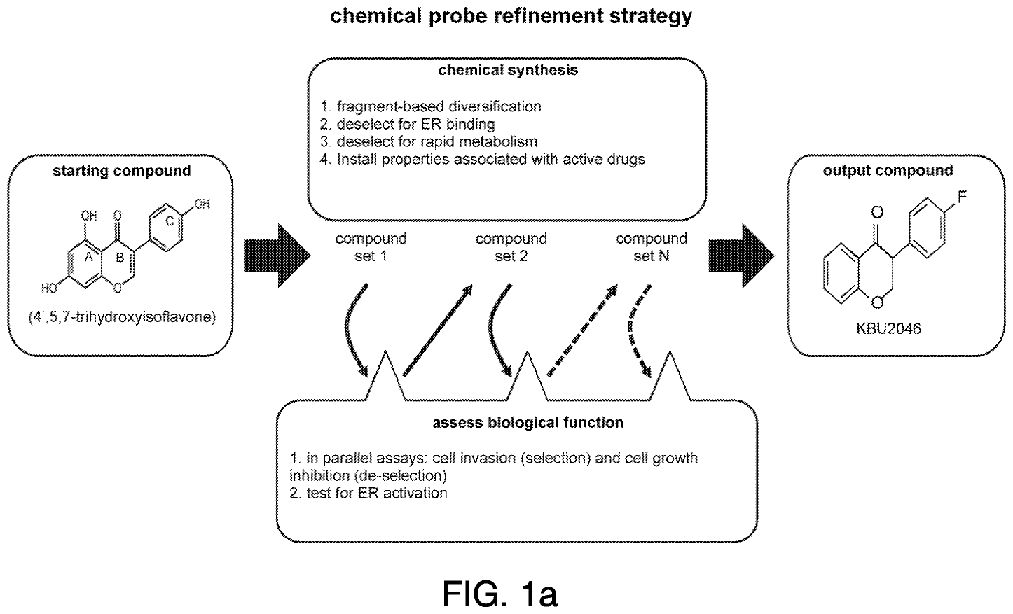

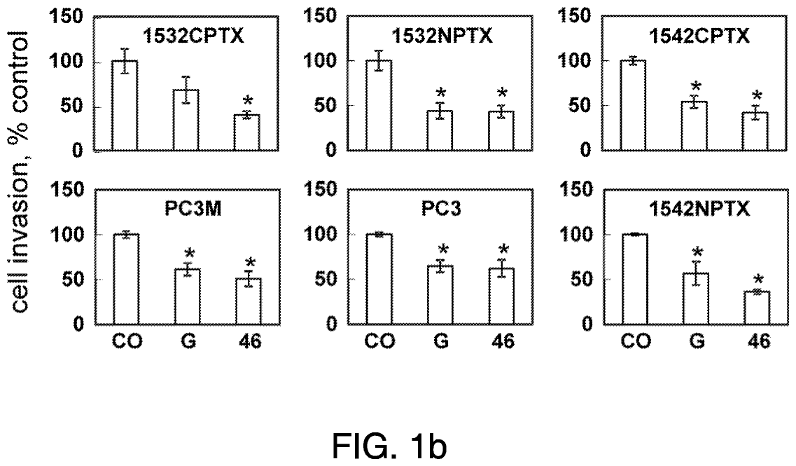

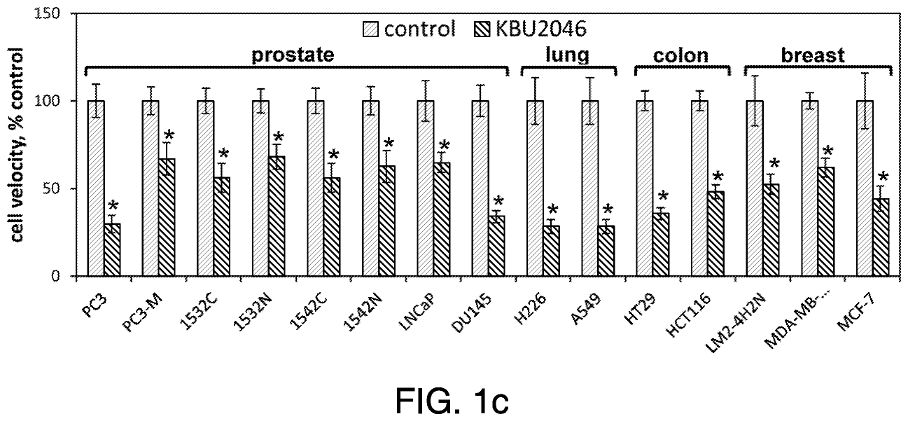

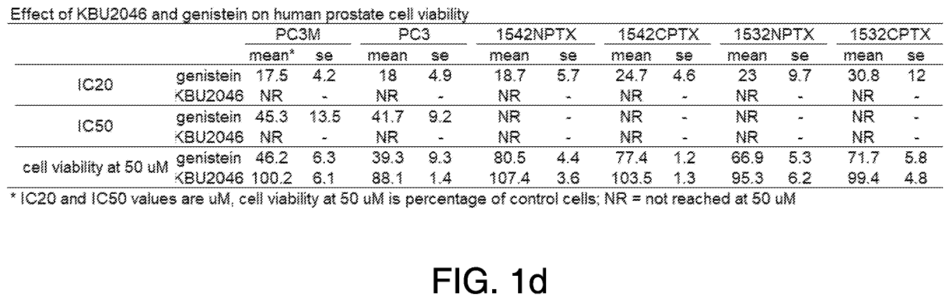

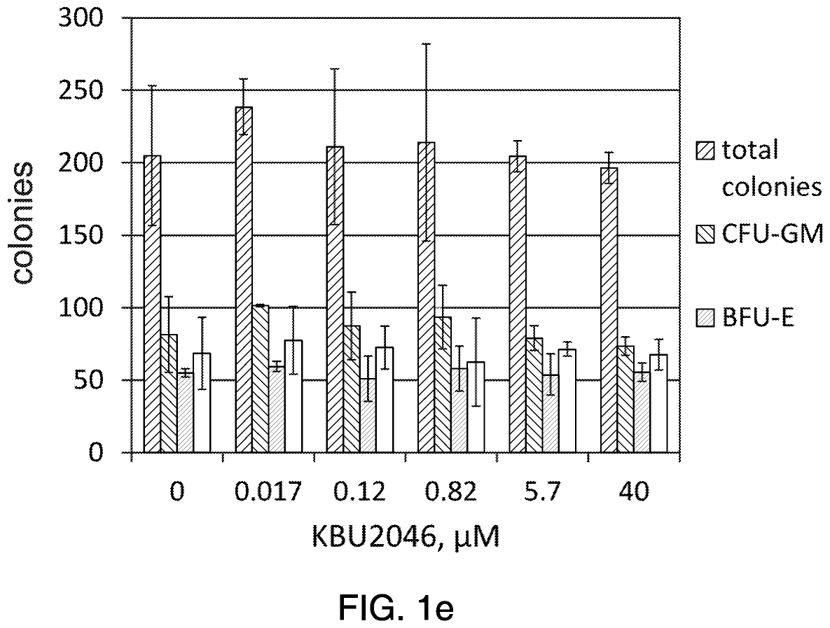

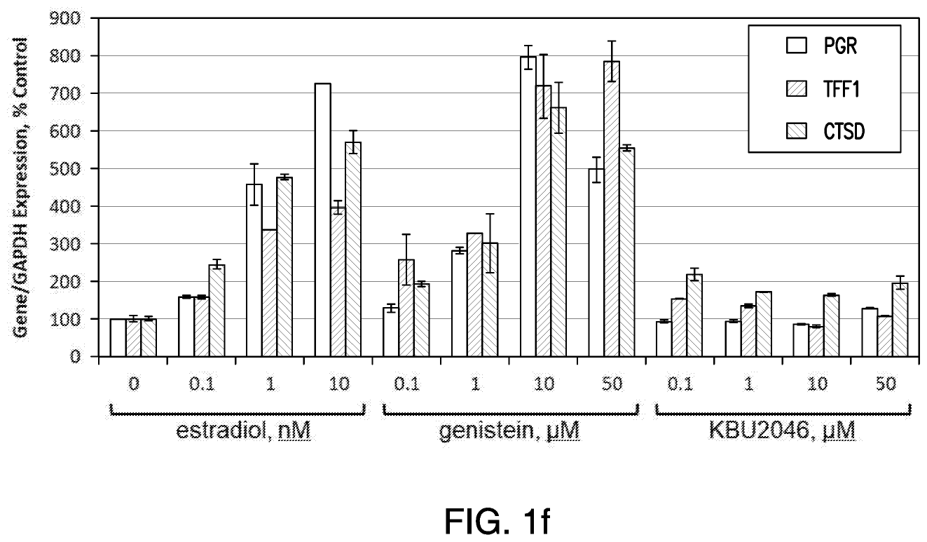

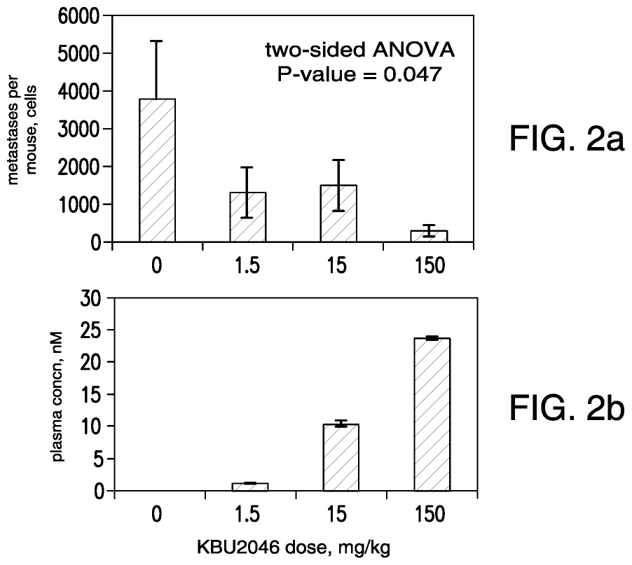

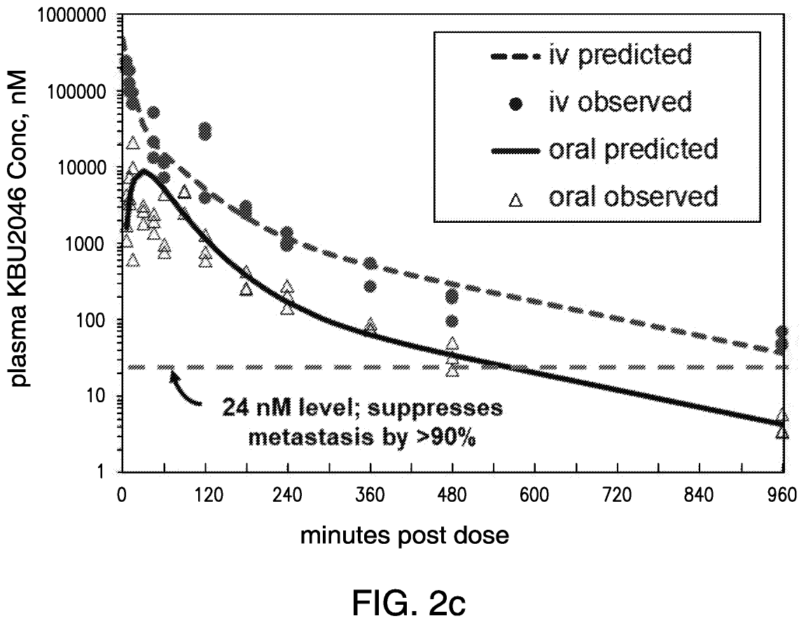

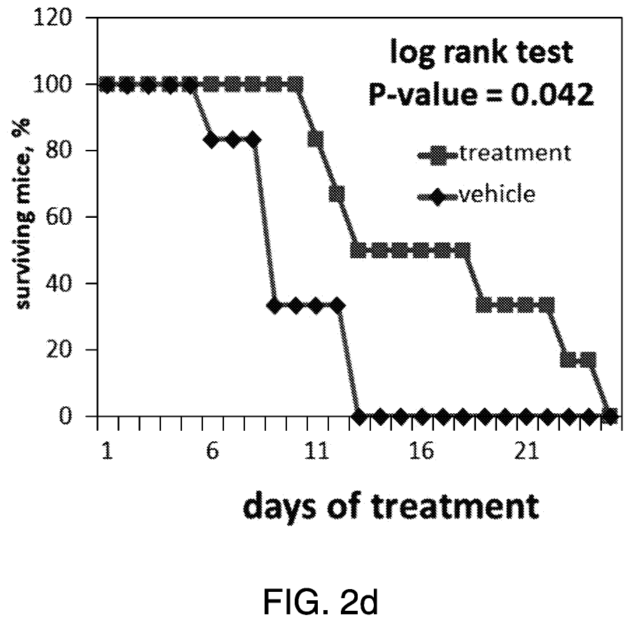

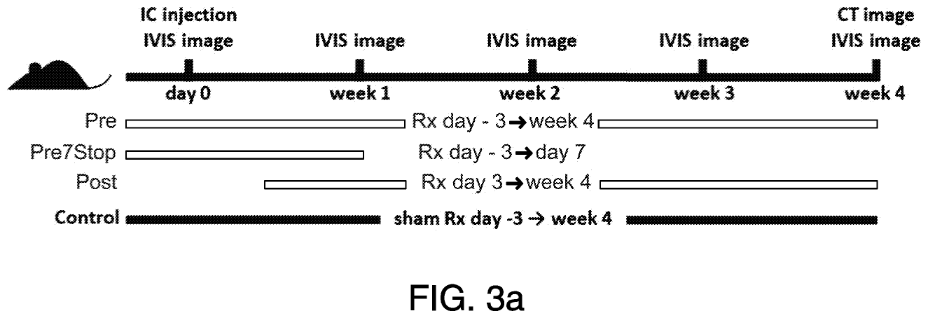

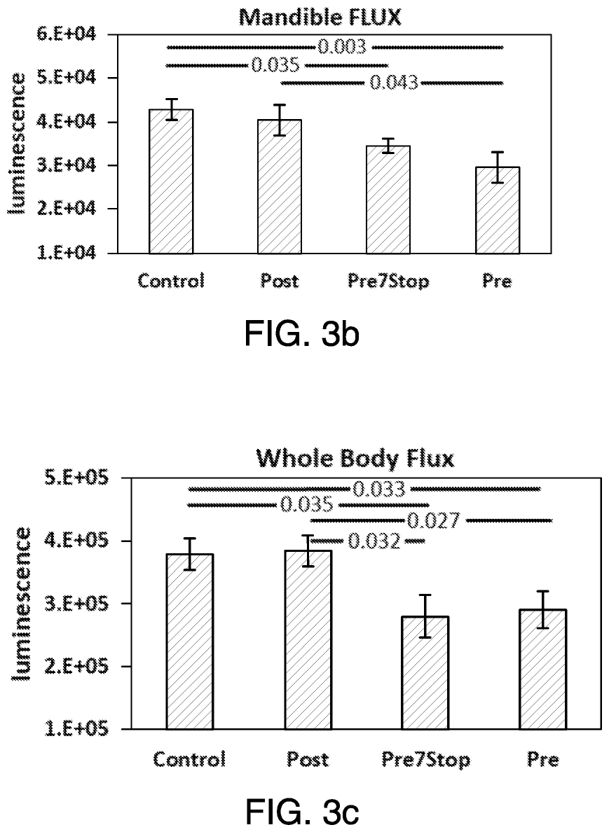

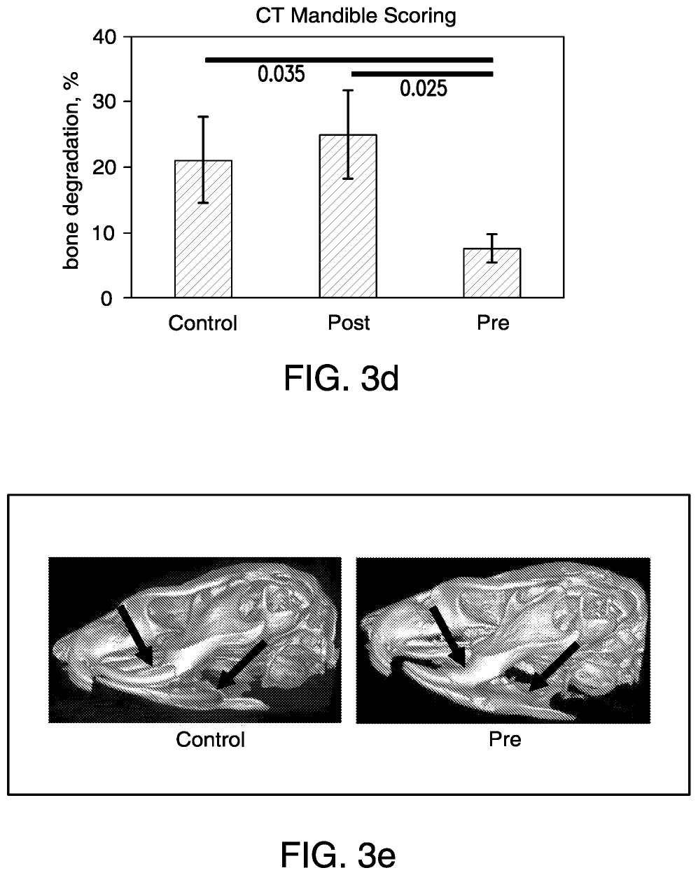

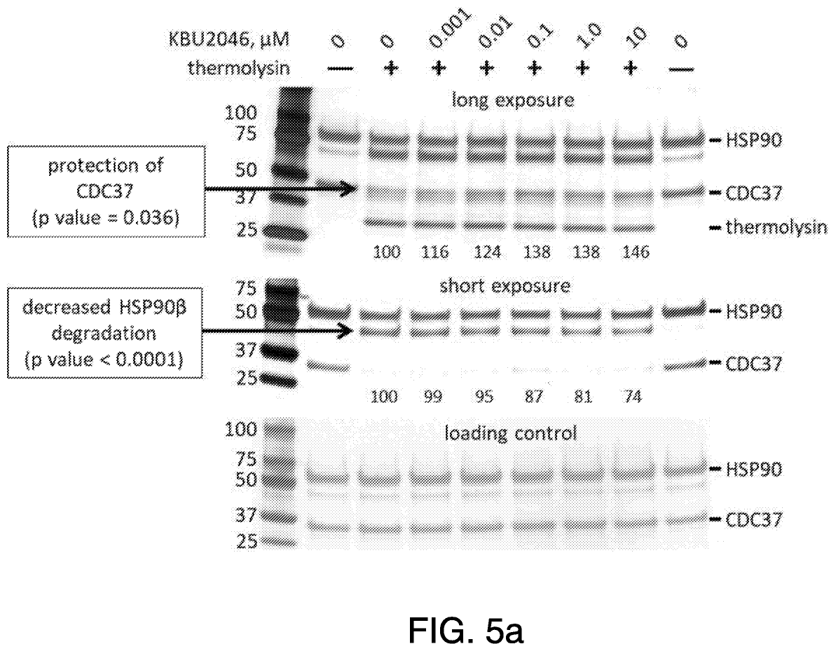

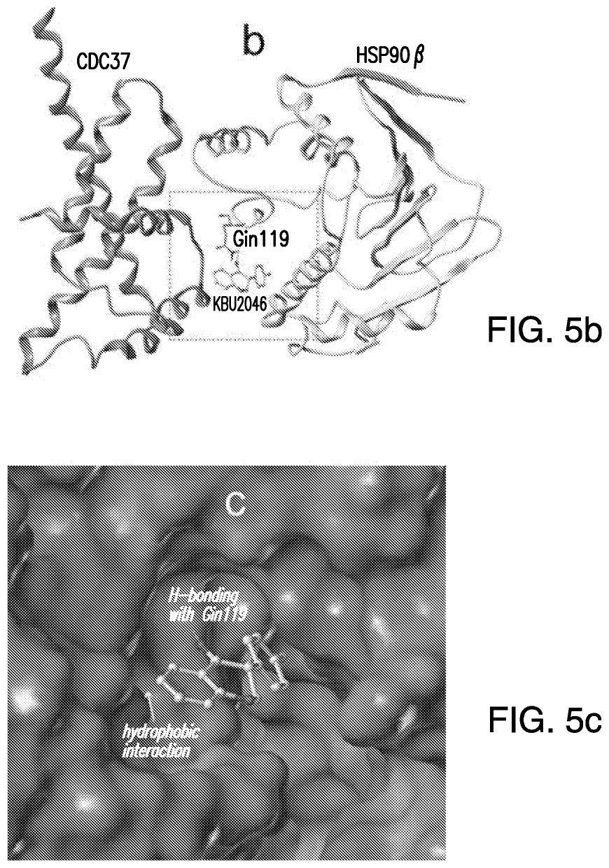

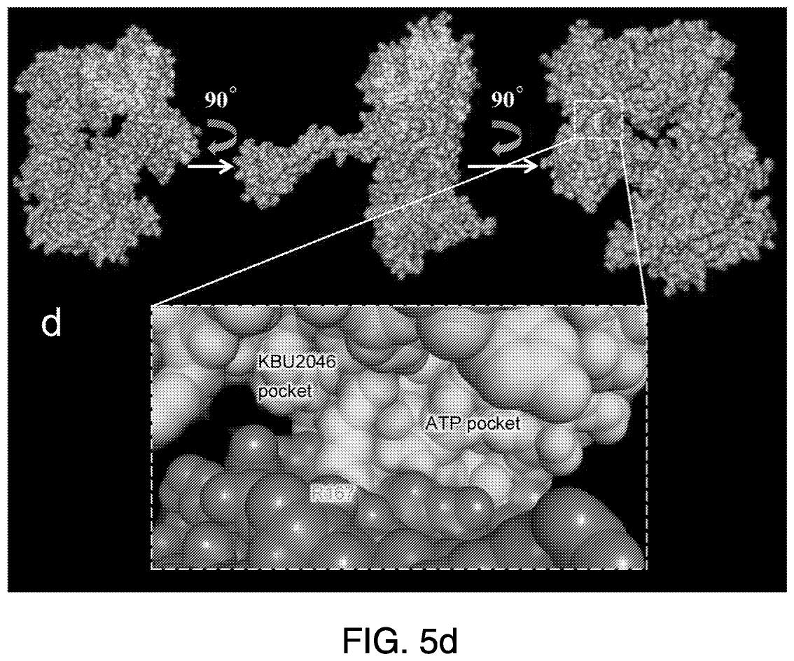

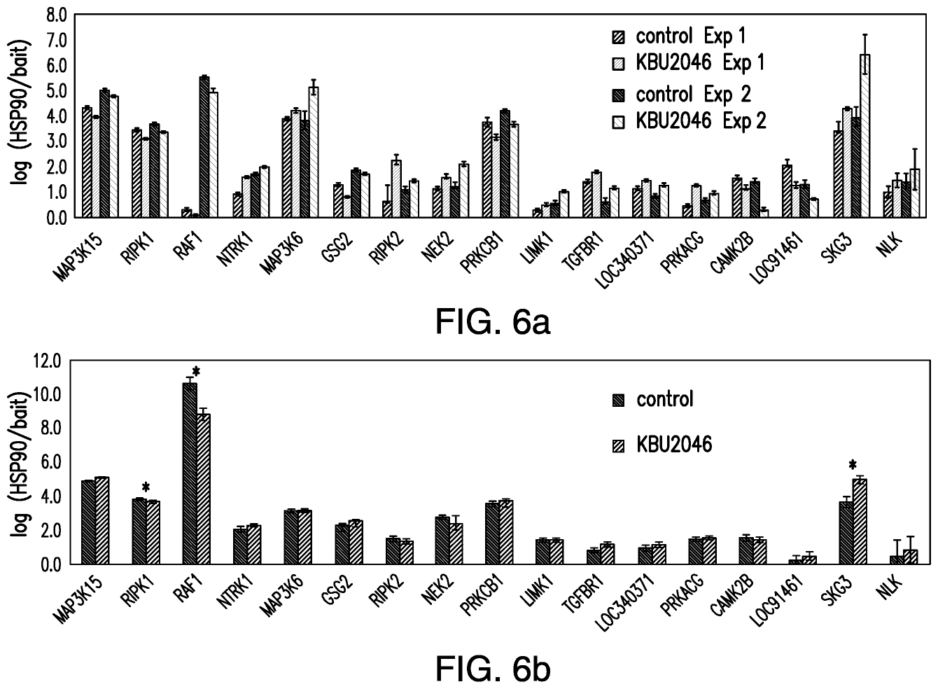

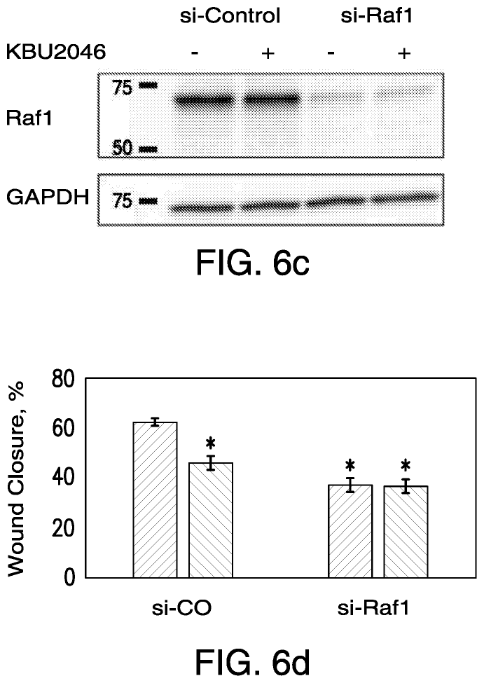

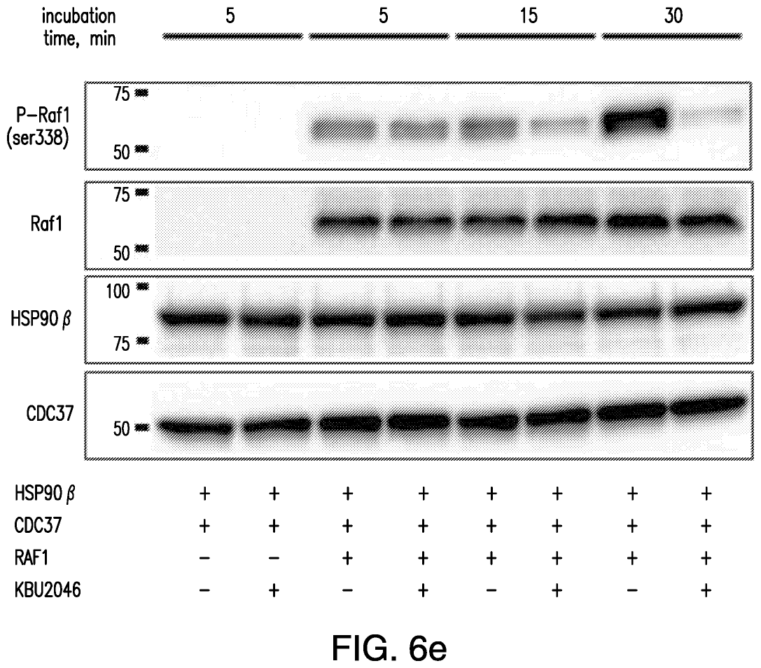

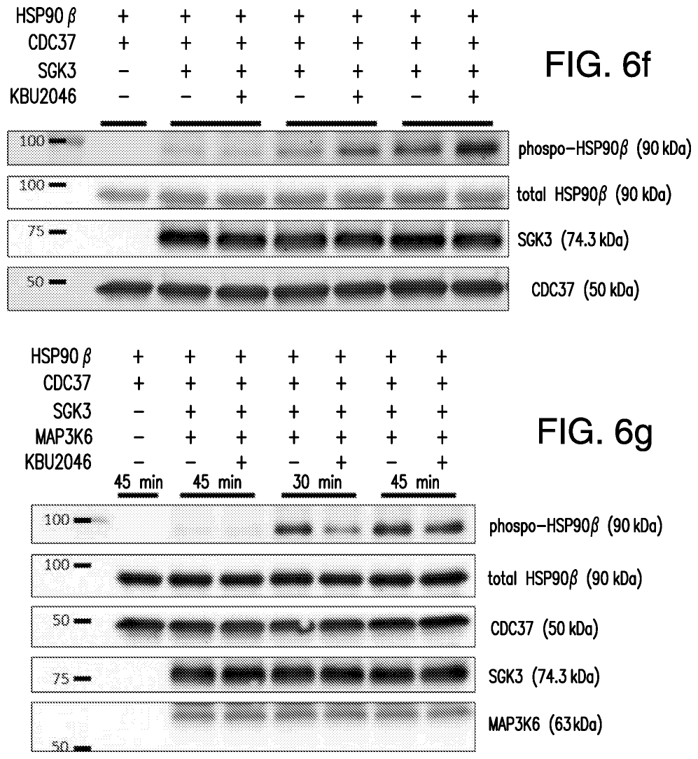

The assay as shown in FIGS. 6e-g demonstrates the ability of the small chemical, KBU2046, to inhibit chaperone-mediated activation of the client protein Raf1. This is important because it demonstrates that this assay can be used to detect biologically important effects on client proteins induced by chemicals. As described herein, the following comprehensive series of findings are demonstrated: i. Raf1 stimulates cancer cells to move, and KBU2046 stops cancer cells from moving by blocking the effect of Raf1 (e.g., FIGS. 6c-d); blocking cancer cells from moving represents an important biological/therapeutic effect because the increased movement of cancer cells is what causes them to move throughout the body, i.e., the spread of cancer, which leads to death; ii. Inside of cells, KBU2046 inhibits HSP90 from binding to Raf1 (see, FIGS. 6a-b); KBU2046 physically binds to a protein complex consisting of HSP90 and CDC37 (known as a co-chaperone), see, for example, FIG. 5; iv. KBU2046 stops human prostate cancer from destroying bone (e.g., FIG. 3), prostate cancer cells spread to bone in humans, form tumors in the bone, destroy the bone, and thereby cause high levels of morbidity and mortality in humans; v. KBU2046 stops human prostate cancer (e.g., FIGS. 2a-b) and human breast cancer (e.g., FIG. 2d) from spreading throughout the body; and vi. KBU2046 stops the movement of at least four different cancer cell types: breast, prostate, colon and lung cancer (e.g., FIG. 1c).

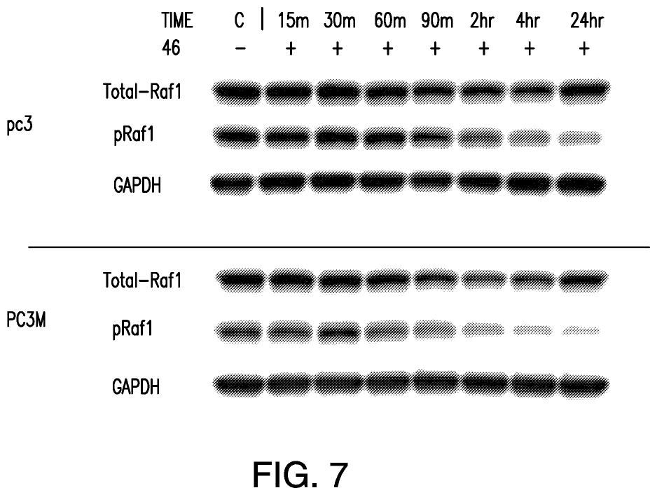

Also described herein and as shown in FIG. 7, is that KBU2046 decreases the activation of Raf1 in human prostate cancer cells. In FIG. 6e, it is demonstrated using the assay disclosed herein that KBU2046 inhibits phosphorylation of Raf1, and that it does so at a specific site, known as the activation motif. Inhibiting phosphorylation at this site, inhibits activation of Raf1. These data provide evidence that the findings demonstrated in vitro assay reflect what is actually happening inside of live cells. Increased cell motility is a fundamental characteristic of cancer cells (Talmadge, J. E. et al., Cancer Res 70, 5649-69 (2010)). It is required in order for cells to invade through the basement membrane, represents an initial step in the metastatic cascade and is important for cells to move from their primary organ of origin to distant metastatic sites. The movement of cancer cells out of their primary organ of origin greatly reduces the chances of survival (Wells, A. et al., Trends Pharmacol Sci 34, 283-9 (2013)). Movement of cells to distant organs, and their resultant destruction, constitutes a primary cause of cancer-associated morbidity and mortality (Minn, A. J. et al., Principals and Practice of Oncology (2008)). Processes that drive the development of increased cell motility represent high value therapeutic targets. However, comprehensive endeavors aimed at selectively inhibiting cancer cell motility and resultant metastasis have met with failure (Steeg, P. S., Nat Med 12, 895-904 (2006); and Krishna et al., Future Med Chem 6, 223-39 (2014)). While many pathways have been shown to regulate cell motility, they constitute pathways whose regulatory effects are pleiotropic (Krishna et al., Future Med Chem 6, 223-39 (2014)). It has therefore not been possible to identify regulators of cell motility possessing the selective capacity to support targeted manipulation.

Recognizing the importance and intractable nature of this problem, it was reasoned that it needed to be approached in an uncommon manner. The approach disclosed herein took into consideration that small chemicals have potent biological properties, that single atom changes in their structure can affect those properties, that chemical structure can be modulated and that as such they constitute highly refined biological probes. It was hypothesized that chemicals could be used to identify novel and selective sites that regulate cancer cell motility and that such sites would constitute high value therapeutic targets.

Herein a novel and selective regulatory mechanism for these processes was delineated using efficient synthesis routes and resultant small chemicals as biological probes. Described herein is the therapeutic potential of the resultant probe, KBU2046, so identified by demonstrating selectivity across comprehensive molecular, cellular and systemic assays. Efficacy of KBU2046 is demonstrated across several different in vitro models and across multiple murine models of human cancer metastasis, which includes decreased metastasis, decreased bone destruction and prolonged survival. Also, comprehensive pharmacokinetic and toxicity studies further support therapeutic potential. Finally, the molecular mechanism and its ability to perturb the novel regulatory process are also characterized.

Increased cancer cell motility constitutes a root cause of end organ destruction and mortality, but its complex regulation represents a barrier to precision targeting. The characteristics of small molecules were used to probe and selectively modulate cell motility. By coupling efficient chemical synthesis routes to multiple up-front in parallel phenotypic screens, it was identified that KBU2046 inhibits cell motility and cell invasion in vitro. Across three different murine models of human prostate and breast cancer, KBU2046 inhibits metastasis, decreases bone destruction and prolongs survival at nanomolar blood concentrations after oral administration. Comprehensive molecular, cellular and systemic-level assays support a high level of selectivity. KBU2046 binds chaperone-co-chaperone complexes (also referred to herein as heterocomplexes), selectively alters binding of client proteins that regulate motility and lacks the hallmarks of classical chaperone inhibitors, including toxicity. A cell motility regulatory mechanism was identified and a targeted therapeutic was synthesized, providing a platform to pursue studies in humans.

Methods

Disclosed herein are methods for identifying an agent of interest that alters binding or activity of a client protein to a chaperone complex, the methods comprising (a) forming a cell-free chaperone complex in vitro with an isolated chaperone protein and a co-chaperone protein; (b) incubating the chaperone complex with a client protein in the presence or absence of the agent of interest; (c) assaying the binding of the client protein to the chaperone complex or activity of client protein in step (b); and (d) determining whether the agent of interest alters the binding or activity of the client protein in step (c) so as to identify the agent of interest that alters the binding or activity of the client protein, thereby, identifying the agent of interest that alters binding or activity of the client protein to the chaperone complex. Also disclosed herein are methods for identifying one or more agents of interest that alters binding or activity of a client protein to a chaperone-co-chaperone complex. In an aspect, the method can comprise: a) forming a cell-free chaperone-co-chaperone complex in vitro with an isolated chaperone protein and a co-chaperone protein; b) incubating the chaperone-co-chaperone complex with a client protein in the presence or absence of the one or more agents of interest; c) assaying the binding of the client protein to the chaperone-co-chaperone complex or activity of the client protein in step (b); and d) determining whether the one or more agents of interest alter the binding or activity of the client protein in step (c). In an aspect, the method can identify one or more agents of interest that alter binding or activity of the client protein to the chaperone-co-chaperone complex. For example, the methods disclosed herein may identify one or more agents of interest in a high-throughput assay or screen. In an aspect, the high-throughput assay or screen can be automated.

Disclosed herein are methods for identifying one or more agents of interest that alters binding or activity of a client protein to a chaperone-co-chaperone complex. In an aspect, the chaperone-co-chaperone complex can be HSP90β/CDC37. In an aspect, the method can comprise: a) forming a cell-free chaperone-co-chaperone complex in vitro with an isolated chaperone protein and a co-chaperone protein; b) incubating HSP90β/CDC37 with a client protein in the presence or absence of the one or more agents of interest; c) assaying the binding of the client protein to HSP90β/CDC37 or activity of the client protein in step (b); and d) determining whether the one or more agents of interest alter the binding or activity of the client protein in step (c), thereby identifying the one or more agents of interest that alter binding or activity of the client protein to HSP90β/CDC37. In an aspect, the client protein can be a kinase. In an aspect, the kinase can be RAF1.

In an aspect, the method can further comprise incubating the isolated chaperone protein or the co-chaperone protein with the one or more agents of interest. In an aspect, the one or more agents of interest do not bind to the isolated chaperone protein in the absence of the isolated co-chaperone protein. In another aspect the one or more agents of interest do not bind to the co-chaperone protein in the absence of the chaperone protein.

For example, in step (b), incubating conditions may permit the client protein's kinase activity. Further, in an aspect, altering the structure of the one or more agents of interest can involve a change of one or more of the functional groups, introducing one or more substituent, a change in the oxidation state, or altering the backbone ring system or a combination thereof.

In an aspect, the altered activity can be the activity of the client protein to the chaperone complex or chaperone-co-chaperone complex. In an aspect, the activity can be kinase activity, phosphatase activity, ligase activity, E3 ligase activity or transcription factor activity or a combination thereof.

Examples of agents of interest include, but are not limited to, small molecules, biological agents, peptides, polypeptides, antibodies or derivatives or fragments thereof, aptamers, peptide nucleic acids (PNAs), nucleic acids, chemical compounds, flavonoid, coumestan, prenylflavonoid, isoflavone, lignan and a substituted natural phenolic compound. The one or more agents of interest as identified may alter cancer cell invasion and motility. In an aspect, the one or more agents of interest can reduce or inhibit cancer cell invasion. In an aspect, the one or more agents of interest can reduce or inhibit cancer cell motility. In a further aspect, the one or more agents of interest can alter the phosphorylation state of a chaperone protein or co-chaperone protein.

In an aspect, the agent of interest can be a kinase or a phosphatase. A kinase is an enzyme that catalyzes the transfer of a phosphate group from a molecule to a substrate via phosphorylation. Protein kinases are one type of kinases, and act on a protein by phosphorylating them on their serine, threonine, tyrosine or histidine residues. Phosphorylation can modify the function of a protein (e.g., increase or decrease a protein's activity, stabilize it or mark it for destruction, localize it within a specific cellular compartment, and it can initiate or disrupt its interaction with other proteins). A phosphatase is an enzyme that uses water to cleave a phosphoric acid monoester into a phosphate ion and an alcohol. Phosphatase enzymes are involved in many biological functions. Phosphorylation (e.g. by protein kinases) and dephosphorylation (by phosphatases) can serve diverse roles in cellular regulation and signaling.

In an aspect, the method can further comprise modifying the one or more agents of interest and repeating steps a) to d). In an aspect, the step of modifying the one or more agents of interest can comprise changing one or more of the functional groups, introducing one or more substituents, changing the oxidation state, altering the backbone ring systems, altering the molecular weight or a combination thereof.

In an aspect, the method can further comprise assaying one or more agents of interest for cell migration, and identifying and/or selecting one or more chemical derivatives having reduced or no cell migration.

In yet a further aspect, the method can further comprise assaying one or more agents of interest for cytotoxicity, and identifying and/or selecting one or more agents of interest having reduced or no cytotoxicity.

Additionally, in another aspect, the method can further comprise assaying one or more agents of interest for inhibiting cancer metastasis, and identifying and/or selecting one or more agents of interest that reduce or inhibit cancer metastasis.

Further, in another aspect, the method can further comprise assaying one or more agents of interest for promoting survival in a cancer xenograft animal model, and identifying and/or selecting one or more agents of interest promoting survival in the cancer xenograft animal model.

In an aspect, the method can further comprise assaying one or more agents of interest for inhibiting organ destruction in an animal, and identifying and/or selecting one or more agents of interest having reduced or no organ destruction property.

In some aspects, the method can further comprise assaying one or more agents of interest for altering phosphorylation of HSP90, and identifying and selecting one or more agents of interest altering phosphorylation of HSP90. In an aspect, the one or more agents of interest inhibit phosphorylation of HSP90. In an aspect, the one or more agents of interest promote phosphorylation of HSP90.

In an aspect, the method can further comprise assaying one or more agents of interest identified for altering phosphorylation of any chaperone, co-chaperone or client protein.

In an aspect, the method can further comprise assaying one or more agents of interest for altering post-translational modification of any chaperone, co-chaperone or client protein. In an aspect, the post-translation modification can be selected from the group consisting of phosphorylation, acetylation, nitrosylation, methylation, ubiquitination, sumoylation, acylation and oxidation.

In an aspect, the method can further comprise incubating an isolated chaperone protein or co-chaperone protein with the one or more agents of interest, wherein the one or more agents of interest does not bind to an isolated protein.

In an aspect, the method can further comprise determining phosphorylation status of chaperone protein or co-chaperone protein. In an aspect, the one or more agents of interest can alter the phosphorylation state of the chaperone protein or the co-chaperone protein. In an aspect, the one or more agents of interest can promote phosphorylation, inhibit phosphorylation, promote dephosphorylation or inhibit dephosphorylation of the chaperone protein or the co-chaperone protein.

Examples of chaperone proteins include, but are not limited to, Hsp100, Hsp104, Hsp110, Hsp90a, Hsp90b, Grp94, Grp78, Hsp72, Hsp71, Hsp70, Hsx70, Hsp60, Hsp47, Hsp40, Hsp27, Hsp20, hspb12, Hsp10, hspb7, Hspb6, Hspb4, HspB1, and alpha B crystallin.

Examples of co-chaperone proteins include, but are not limited to, Cdc37/p50, Aha1, auxilin, BAG1, CAIR-1/Bag-3, Chp1, Cyp40, Djp1, DnaJ, E3/E4-ubiquitin ligase, FKBP52, GAK, GroES, Hch1, Hip (Hsc70-interacting protein)/ST13, Hop (Hsp70/Hsp90 organizing protein)/STIP1, Mrj, PP5, Sacsin, SGT, Snl1, SODD/Bag-4, Swa2/Aux1, Tom34, Tom70, UNC-45, and WISp39.

Further, an example of a chaperone-co-chaperone complex include, but is not limited to, Hsp90b-Cdc37. Additionally, examples include a chaperone-co-chaperone grouping including any of the chaperone protein of Hsp100, Hsp104, Hsp110, Hsp90a, Hsp90b, Grp94, Grp78, Hsp72, Hsp71, Hsp70, Hsx70, Hsp60, Hsp47, Hsp40, Hsp27, Hsp20, hspb12, Hsp10, hspb7, Hspb6, Hspb4, HspB1, and alpha B crystallin.

In an aspect, the client protein is selected from the group consisting of kinases, phosphatases, ligases, E3 ligases and transcription factors. In an aspect, the client protein can be a polypeptide. In an aspect, the polypeptide can participate in cell motility, cytotoxicity, metastasis, survival, organ destruction, phosphorylation of HSP90beta, covalent modifications of chaperone proteins and/or a co-chaperone. Examples of client proteins include, but are not limited to, MAP3K15, RJPK1, RAF1, NTRK1, MAP3K6, GSG2, RIPK2, NEK2, PRKCB1, LIMK1, TGFBR1, LOC340371, PRKACG, CAMK28, OC81461, SGK3, NLK, and a fragment or derivative thereof. Additional examples include, but are not limited to, the following client proteins as shown in Table 1.

TABLE 1
Examples of client proteins.

| HGNC | HGNC | HGNC | HGNC | HGNC |
|---|---|---|---|---|
| symbol | symbol | symbol | symbol | symbol |
| AMHR2 | FRK | TBK1 | PRKCA | IGF1R |
| ICK | PRKY | PRKCB | CDK18 | TAOK3 |
| PSKH1 | MOS | WNK4 | GRK7 | ROR2 |
| FYN | MAP3K15 | SRPK1 | EPHA1 | PRKCZ |
| EIF2AK2 | TSSK6 | ERBB2 | ILK | PTK6 |
| ARAF | MYO3B | STYK1 | CHEK1 | ERBB4 |
| FER | MAP2K5 | HCK | PRKCG | PASK |
| PSKH2 | SGK2 | AURKC | GRK6 | CDK14 |
| PKN2 | GSK3A | BTK | EPHA2 | MUSK |
| PINK1 | INSRR | PRKG2 | SRPK3 | PTK2 |
| RAF1 | ALPK1 | CDK4 | PIM3 | PRKD1 |
| MAST2 | GRK4 | MAP2K7 | CDK11B | STK32C |
| PRKACB | SGK1 | MAPK15 | TESK1 | MAP4K2 |
| CDK15 | TNNI3K | LYN | LIMK1 | IKBKE |
| LIMK2 | TSSK2 | PRKX | NUAK2 | AURKB |
| MAP3K14 | TNK2 | JAK1 | FGR | PRKCI |
| MAP3K8 | AXL | TP53RK | PRKCH | PTK2B |
| MERTK | ERBB3 | TSSK3 | MAP4K1 | MINK1 |
| DYRK4 | FGFR1 | RPS6KA5 | MYLK3 | NEK9 |
| LCK | MATK | HIPK4 | RET | DAPK3 |
| TIE1 | ITK | MAPK4 | MAP3K9 | CAMKK2 |
| NPR2 | CLK3 | YES1 | CDK6 | AKT2 |
| CDK9 | NTRK3 | MYLK2 | ALK | PAK6 |
| MAPK7 | EIF2AK1 | BLK | RPS6KB1 | CAMK4 |
| FGFR3 | FLT4 | SGK223 | RPS6KC1 | PRKD2 |
| TESK2 | CSNK1A1 | IRAK2 | PRKAA2 | RPS6KA1 |
| PRKAA1 | ACVR1B | STK38 | BRAF | CAMK2A |
| MAP3K6 | DCLK2 | TSSK1B | MAP3K5 | CAMK2B |
| TNK1 | NTRK2 | PRKCE | RPS6KA2 | CAMK2D |
| EPHB6 | STK11 | NEK8 | STK38L | CLK2 |
| CDK7 | MAP4K4 | CAMK2G | FES | CDKL4 |
| CSF1R | NTRK1 | DYRK1B | RPS6KA6 | BUB1B |
| MAP3K12 | ACVR2B | PDGFRB | RPS6KA3 | MAP4K5 |
| EPHB1 | RIPK1 | PDIK1L | EPHA4 | BMPR2 |
| ACVR1C | SGK3 | PRKCQ | IRAK3 | SNRK |
| DDR2 | MAP3K2 | TYRO3 | DMPK | ULK2 |
| FASTK | ABL1 | RPS6KL1 | CAMKV | UHMK1 |
| MYLK4 | TYK2 | PKN1 | CAMK1G | CDK20 |
| KSR2 | CAMKK1 | CDK3 | PIM2 | TLK1 |
| BMPR1A | NEK11 | DDR1 | BMX | PDGFRA |
| | KSR1 | DYRK2 | STK32B | SLK |
| MAPK9 | CDK10 | PAK1 | KBTBD4 | NHLRC1 |
| STK33 | TRIB3 | MAPK13 | FBXL2 | SF3B3 |
| PLK4 | CSNK1E | SRPK2 | FBXL12 | KBTBD7 |
| RIOK1 | STK19 | SRC | KLHL6 | FBXO3 |
| TLK2 | NRBP2 | MAPK14 | ENC1 | RAB40A |
| ADCK1 | CDK1 | NEK6 | KCTD8 | RNF10 |
| MAP4K3 | CHEK2 | PXK | GAN | ANAPC2 |
| STK32A | ERN1 | STK24 | KLHL25 | KIAA0317 |
| EGFR | CSNK1D | EPHA10 | LOC440248 | KLHL15 |
| RIOK2 | STK16 | MAPK12 | FBXL14 | WWP1 |
| PRKAA1 | SIK1 | VRK1 | FBXW7 | PCGF1 |
| DSTYK | TGFBR1 | SYK | FBXO9 | HECTD3 |
| ZAK | CSNK2A2 | SGK196 | KLHL22 | TRIM49 |
| PLK2 | MKNK1 | CAMK1 | PRPF19 | KLHL1 |
| ACVRL1 | CDK19 | CDK16 | KLHL26 | TRIM10 |
| ACVR1 | AKT1 | PRKG1 | RAPSN | KLHL23 |
| RIPK2 | GRK6 | EEF2K | FBXO18 | TRIM2 |
| SIK2 | TRIB1 | MAPKAPK5 | FBXL15 | ZBTB20 |
| RPS6KA4 | OXSR1 | PLK1 | KLHL10 | KLHL29 |
| CSNK1A1L | NRBP1 | MAP2K6 | FBXL8 | KLHL32 |
| CSNK1G1 | SCYL3 | CSK | TRIM56 | SH3RF2 |
| MARK3 | NLK | STK40 | TRIM17 | FBXO38 |
| LMTK2 | NEK7 | MAPKAPK3 | FBXL18 | LGALS3BP |
| PRKACG | MAPKAPK2 | MAP2K2 | KLHL36 | ASB3 |
| GSG2 | CSNK1G2 | STK25 | LNX1 | CUL2 |
| PKMYT1 | ZAP70 | DAPK2 | RNF19B | KLHL13 |
| AKT3 | PRKACA | PAK4 | ASB4 | PCGF3 |
| CDK10 | STK3 | BRSK2 | RNF10 | HERC6 |
| NEK3 | PAK2 | FBXW2 | BTRC | RHOBTB1 |
| CDK17 | PHKG2 | CUL3 | KLHL34 | FBXO40 |
| CDK11A | PIM1 | CUL4B | ARMC5 | FBXW5 |
| ADCK4 | MAPK1 | FBXO38 | DET1 | KCNG1 |
| AURKA | CDK2 | FBXO25 | KLHL14 | SPSB3 |
| CASK | CDK5 | SKP2 | VPS18 | TRIM36 |
| MAPK8 | ADRBK1 | TRIM56 | FBXO28 | ZBTB17 |
| DAPK1 | CAMKID | KLHL38 | FBXO17 | DTX4 |
| MARK1 | PAK7 | FBXO24 | RFWD3 | ASB6 |
| PDPK1 | TRIB2 | FBXL3 | FBXO27 | FBXO6 |
| PBK | MAPK3 | FBXW11 | KLHL36 | ZNF509 |
| RIOK3 | BMP2K | WSB2 | FBXL13 | MARCH9 |
| PLK3 | MAP2K3 | | ASB2 | LGALS3BP |
| LRSAM1 | TRIM41 | KCNS2 | KLHL20 | TRIM9 |
| FBXO4 | WWP2 | ARIH2 | TRIM21 | FBXL20 |
| TRIM32 | KCTD13 | RFPL4B | CBLL1 | TRIM72 |
| TNFAIP3 | TCEB3B | ASB13 | RNF212 | KCTD15 |
| PARK2 | RNF213 | ZNF238 | BMI1 | TRIM47 |
| TRIM74 | TRIM49 | RNF146 | ANUBL1 | TRIM48 |
| RCBTB2 | TNFAIP3 | RNF139 | KBTBD2 | PCGF6 |
| TRIM36 | LNX1 | KCNA2 | RNF183 | PCGF5 |
| RCBTB1 | RAB40B | SYVN1 | RNF13 | ZBTB37 |
| VPS41 | TRAF2 | RNF212 | KBTBD10 | XIAP |
| ASB17 | RNF214 | RHOBTB1 | STUB1 | KBTBD3 |
| KCNA5 | MGRN1 | TRIM69 | RBCK1 | MID2 |
| CUL1 | KCNG3 | ASB9 | TRIM35 | ASB10 |
| PCGF6 | RNF31 | UBE3B | BCL6B | BTBD1 |
| KCNS3 | BTBD12 | DPF1 | TRIM38 | ZBTB7B |
| TRIM73 | VHL | RHOBTB3 | RNF128 | KCTD4 |
| SPSB1 | TRIM52 | LONRF1 | ZBTB5 | ZBTB45 |
| KCNA6 | KCND2 | UBR2 | GMCL1 | LNX2 |
| SOCS6 | ABTB1 | ZNF131 | HERC3 | SPRYD5 |
| HERC4 | RBCK1 | KCND3 | KBTBD8 | TRIM54 |
| TRIM41 | KCNS3 | FBXO38 | SKP1 | FBXO32 |
| TRIM7 | KCTD3 | RSPRY1 | ASB9 | RNF145 |
| G2E3 | TRIM9 | ZSWIM2 | RNF103 | FBXO46 |
| RNF111 | UBE3A | KEAP1 | RNF8 | MARCH6 |
| RNF40 | TRIM15 | BIRC3 | BIRC2 | SPSB4 |
| MDM4 | TRIM39 | KCTD14 | RNF126 | SF3B3 |
| FBXO34 | BRCA1 | UBOX5 | MYNN | RNF133 |
| FBXO10 | FBXO15 | GTF2H2 | RING1 | FBXO22 |
| TRIM37 | LZTR1 | RNF150 | IPP | RNF185 |
| ZBTB40 | UHRF1 | MARCH11 | KCNA3 | UBE3C |
| FBXO5 | PML | KBTBD5 | ZBTB9 | ZBTB44 |
| ASB7 | TRIML1 | TRIM43 | KCTD16 | ZNRF1 |
| UBE3B | BTBD9 | ZBTB8 | RNF6 | RNF175 |
| BACH1 | ZBTB16 | RNF126 | UBE3C | ZBTB26 |
| CBLC | DTX2 | GTF2H2 | RNF135 | RNF170 |
| SOCS5 | TRIM22 | TRAIP | KCND3 | ZNF645 |
| KCND1 | RNF180 | RNF38 | ZBTB22 | FBXO32 |
| RNF14 | SOCS2 | TRIM59 | TRAF4 | ZNF645 |
| TCEB3B | RNF26 | FBXO30 | PJA1 | SKP1 |
| TRIM39 | PHF7 | RNF219 | MYLIP | KLHL28 |
| FBXO7 | TNFAIP1 | PPIL2 | ZBTB43 | RNF166 |
| KCNC1 | CUL4A | CGRRF1 | KCTD17 | RGS6 |
| TRIM55 | RBX1 | FBXO8 | SH3RF2 | ZBTB20 |
| SPOPL | PPIL2 | RNF182 | C17orf65 | CUL3 |
| TRIM27 | RC3H2 | FBXO2 | RNF113A | PREB |
| RNF7 | MKRN3 | RNF32 | RNF141 | ZBED4 |
| ZBTB44 | KLHL17 | KCTD17 | RNF138 | RAG1 |
| RBBP6 | RNF181 | CCNB1IP1 | TRIP12 | ZC3H7B |
| FBXL5 | ZBTB25 | RNF115 | UBR1 | FOXD4L6 |
| TRAF5 | TRIM23 | FBXW4 | HECW2 | RGS9 |
| ZNF509 | SOCS4 | RNF41 | DZIP3 | NR3C2 |
| RNF151 | KLHL12 | RNF130 | FBXO18 | POGK |
| ZFAND5 | MNAT1 | KCTD6 | FBXO18 | NR1H3 |
| LOC83459 | BIRC8 | RNF144B | PCGF5 | RGS7 |
| PCGF2 | RABGEF1 | KCNRG | RAG1 | PRDM1 |
| RNF7 | DTX3 | PEX10 | ZBTB46 | STAT2 |
| FBXO44 | RNF114 | NOSIP | MEX3D | NR2C2 |
| MID1 | RNFT1 | TRIM5 | KLHDC5 | PRDM1 |
| TRIM60 | KCTD9 | MGC23270 | MUL1 | CUL4B |
| RNF11 | SOCS3 | TRIM11 | ZBTB24 | GTF2IRD2 |
| TCEB2 | RNF5 | CDCA3 | RNF113B | DLX6 |
| ZFAND3 | SIAH1 | RNF32 | FBXO36 | NR1I3 |
| ZBTB38 | ZBTB6 | RCHY1 | FBXL10 | HP1BP3 |
| ASB14 | PEX10 | ASB11 | CBLB | SREBF1 |
| ZFP161 | SPSB2 | ZBTB44 | RNF20 | NFRKB |
| RNF5 | KCTD5 | NOSIP | BARD1 | KAT5 |
| MARCH3 | KLHL32 | RNF2 | UBE4A | IRF2 |
| BFAR | RLIM | RNF125 | FBXO34 | TFDP3 |
| ZBTB48 | NEURL3 | PXMP3 | PJA2 | TEAD2 |
| KLHL18 | RFFL | TRIM13 | FBXO42 | TEAD2 |
| BTBD10 | TRIM31 | KCTD1 | BRCA1 | C20orf194 |
| ZBTB10 | ZFAND6 | ZBTB32 | ASB8 | LARP4B |
| RFPL3 | MKRN3 | PCGF3 | TRIM42 | ZNF74 |
| KLHL28 | RNF144A | RNF111 | BARD1 | SIM2 |
| TRIM31 | FBXL18 | TRIM65 | DTX3L | NR1I2 |
| CISH | RNF220 | RCHY1 | LONRF3 | HMGA1 |
| KLHL7 | CUL1 | KLHL3 | ASB6 | TBX22 |
| RNF166 | VPS11 | BIRC7 | FBXO11 | BBX |
| MKRN2 | RNF24 | TRIM4 | CUL5 | ANAPC2 |
| RNF34 | FBXO8 | BIRC8 | CUL4A | CXXC1 |
| TRIM27 | LRRC29 | SIAH1 | RGS11 | DMRTA1 |
| ZBTB24 | RNF25 | WWP2 | | SETDB1 |
| NFIC | DLX2 | FOXM1 | MEOX1 | WT1 |
| SLFN11 | DEK | PKNOX1 | DMRT3 | ZSCAN12 |
| ZNF215 | SETDB2 | CUL1 | ZNF174 | MEIS2 |
| TCF25 | BRD9 | NEUROD1 | TRPS1 | ART5 |
| ISX | RFX5 | ZNF384 | HEYL | ZNF3 |
| TADA2A | SATB2 | MATR3 | THAP8 | SSH2 |
| ESR1 | TIGD7 | TFDP1 | FOXC2 | SOX15 |
| ATF3 | ZNF451 | PRDM14 | MAX | SNAI1 |
| THAP4 | CEBPG | LHX6 | MBD4 | ZNF333 |
| PPARD | TBX15 | RORC | HMG20B | ELF4 |
| CEBPE | ZNF333 | TFAM | POLR2L | PITX2 |
| FOXP2 | DVL2 | YEATS4 | EIF3K | ZBP1 |
| MKX | PITX1 | DVL1 | MYT1 | ZNF69 |
| MAX | HIST1H1T | ZNF512 | ZNF616 | USF1 |
| TP53 | PAX7 | CDC5L | TRIT1 | CAMTA2 |
| FOXM1 | MEOX2 | ZKSCAN1 | MIER2 | HESX1 |
| MAFG | HMGA1 | ZNF79 | SNRPN25 | SIX2 |
| GCM2 | DLX4 | ZFP28 | TADA2A | MESP2 |
| DMRTA1 | BARX2 | ZNF551 | ZBTB5 | HOXC11 |
| DNAJC2 | IRF3 | THRA | ATF2 | GTF2E2 |
| ETV7 | NR4A1 | NFIL3 | LEF1 | ZNF366 |
| ARNTL2 | ETV6 | MAX | NR5A1 | ZKSCAN2 |
| ZDHHC19 | CREB3 | HMGB1 | FOXN3 | GATA2 |
| TEF | ERF | NFATC1 | HLX | HMG20B |
| MECP2 | HOXC10 | OTX2 | MBNL2 | ELK3 |
| ZFPM2 | CBLL1 | MSC | CREB3 | TIGD6 |
| RFX3 | UNKL | ZNF488 | USP39 | ZNF497 |
| MAX | HOXD3 | E2F8 | SIX4 | PCGF6 |
| ZNF512B | DUS3L | VSX2 | TBX18 | FMNL2 |
| HOXA9 | SOX14 | LARP6 | PRR3 | ZNF557 |
| TEAD1 | MAX | ARNTL2 | HOXD10 | ZBTB43 |
| PCGF6 | HMGB4 | MEF2B | ELK1 | ETV4 |
| KAT7 | DLX1 | ZNF555 | ZNF699 | HIST1H1E |
| ZNF843 | DUS3L | ZNF434 | SALL2 | THAP7 |
| OTX1 | CHST12 | CSDA | CREB5 | FOXO4 |
| VAX2 | CREB3L1 | CDX4 | FOXJ2 | ZNF606 |
| IRF9 | CREM | DEPDC5 | DMRTB1 | FOSL2 |
| TBX10 | ELF5 | MLLT3 | RBPJ | USF2 |
| LMX1B | NR4A2 | NANOGP8 | GOLGA2 | TLX2 |
| FAM170A | MTA1 | NHLH1 | MEF2C | DMTF1 |
| H2AFX | ELF1 | NR2E1 | ZNF449 | CPSF4 |
| TFAP4 | OVOL1 | HMX2 | ZKSCAN3 | ZBTB37 |
| IFI16 | DMC1 | ZNF57 | SOHLH2 | E2F6 |
| REL | MESP1 | TERF2IP | ZC3H18 | ZSCAN2 |
| ZNF626 | MXD3 | GATA2 | ZNF517 | ZNF343 |
| ZNF281 | HOXA5 | ZNF224 | KIAA0415 | IGHM |
| FARSB | ZFP36L1 | MTA1 | LIN28A | HOXB6 |
| FOXP4 | HIST1H1A | CUL2 | RHOXF1 | ZNF800 |
| BCL6 | ARNT | VPS72 | ZNF428 | ZNF581 |
| KLF7 | THAP1 | ZBTB24 | JUN | VSX1 |
| CDX2 | MITF | THAP10 | RELA | DLX5 |
| EZH2 | ZKSCAN1 | ISL1 | ZNF341 | MEF2D |
| IKZF3 | TIGD5 | ZMAT3 | HMGB2 | ZZZ3 |
| ZNF141 | FOXP3 | AGXT2L2 | KLF11 | ZNF321 |
| DVL3 | ZNF639 | POLE4 | ZRSR1 | HOXA1 |
| ZBTB44 | GRHL2 | ZBTB44 | ZNF195 | PBX4 |
| THAP8 | GATAD1 | ZFP64 | FOXO3 | ZNF596 |
| ZNF529 | ZNF768 | NFE2L2 | ZNF343 | HOPX |
| ZNF687 | ZNF202 | CERS4 | LHX9 | KIAA1683 |
| NR0B1 | ESYT1 | RELL2 | SETDB1 | SHOX2 |
| FARSA | AKAP8L | RAPGEF4 | ZNF638 | HOXB13 |
| ZNF544 | STAT6 | SOX5 | ZNF503 | ZHX3 |
| GMEB1 | MEF2A | ZNF396 | SSH3 | MRPL28 |
| TRIM32 | NOC4L | RNF125 | POU5F1 | ZKSCAN4 |
| EGR3 | ZNF488 | HIST1H1C | ZBTB48 | MNT |
| TOX2 | TRMT1 | MXD4 | ARHGAP35 | ZNF155 |
| POU4F3 | ZNF500 | GLIS3 | ZNF652 | HEY1 |
| GATAD2B | HLF | MRPL28 | ZNF34 | ATMIN |
| ZFP36 | ZNF192 | ZNF205 | ZNF238 | ZSCAN1 |
| ZXDC | MRRF | ZBTB7B | ZNF343 | TGIF2LY |
| IKZF2 | LHX4 | NFAT5 | NUPL2 | NKX2-5 |
| LCORL | ZNF554 | ASCL4 | ZNF300 | DMRTC2 |
| LHX8 | HOXC9 | ATOH1 | ZNF536 | ZFP161 |
| NFKB1 | NFIA | NUFIP1 | HOMEZ | LARP1 |
| HOXB7 | ZNF425 | ZNF593 | MIER1 | ZNF217 |
| NFIB | PRDM5 | NFYB | ZNF324 | ZNF75D |
| NR1H3 | ARNTL | ZCCHC6 | SMAD4 | ZBTB6 |
| TGIF1 | ZRSR2 | RFX3 | ZNF175 | TRIM23 |
| SMAD5 | ZNF219 | ZBTB16 | ZBTB49 | ZNF131 |
| RFX6 | HAND2 | PITX1 | ZNF483 | STAT3 |
| ZNF750 | ZNF24 | ETS2 | RBM26 | GATA2 |
| TIGD4 | ZNF184 | RARA | TSHZ3 | ZNF346 |
| MBD4 | MLX | ZNF641 | HP1BP3 | ZNF684 |
| RNF138 | ZNF165 | KRTAP5-9 | ZNF582 | IGHM |
| RBM26 | ZSCAN21 | FOSL2 | OSR2 | ZNF187 |
| ZNF428 | ZNF658 | FLH3 | POU2F2 | TRAFD1 |
| ZNF3 | ZNF44 | ZNF3 | ZNF765 | C17orf49 |
| MBD3 | ZNF193 | SCAPER | SP6 | ESRRA |
| GATA3 | BOLA1 | PRKRIR | TSC22D4 | CREB3L1 |
| ZNF225 | ZNF496 | ZNF655 | POU5F1 | LHX5 |
| ZIM3 | PLEKHB1 | CERS3 | PMS1 | ZNF219 |
| ZNF221 | SATB1 | MSX2 | ZSCAN22 | RBM6 |
| SNAI3 | THRB | LARP1B | ZZZ3 | ZNF519 |
| ZNF169 | SPDEF | ZFP64 | RXRG | PLXNA4 |
| NEUROG1 | ZNF329 | TSC22D3 | HLA-DRB3 | ZNF414 |
| RFXANK | ZNF707 | ZNF277 | NR1D2 | FERD3L |
| NEUROG3 | TRIT1 | PRDM7 | TCF25 | SMARCC2 |
| SPIB | ZBTB49 | EBF3 | ZNF649 | ZNF772 |
| ZNF621 | SPIC | ZNF521 | JUNB | ZNF423 |
| GTF2F1 | TFAP2A | DDIT3 | ZFYVE26 | SMARCA5 |
| TFEB | IRF8 | AFF4 | KLF3 | CHD2 |
| ZNF770 | ARNT2 | CHRAC1 | MXRA8 | MBNL3 |
| ZNF85 | ETS2 | U2AF1L4 | SOX10 | ZNF394 |
| PRDM15 | CDX1 | NFATC3 | ZNF679 | EHF |
| IKZF1 | NR0B2 | RFX2 | RBM5 | FAM171B |
| ZNF169 | TCF19 | ZNF207 | IGHM | ZNF774 |
| EWSR1 | VDR | ZNF436 | DPF2 | FOXP2 |
| ZNF627 | VENTX | FLI1 | ZNF697 | SALL4 |
| ZBTB25 | PAX4 | BACH1 | ZNF581 | PAX3 |
| ZNF133 | EXOC2 | PLEKHA4 | FOXP1 | ZNF696 |
| MAEL | TOX | CHD1 | EDF1 | CRX |
| NFYA | ZNF396 | ZNF688 | ZNF563 | GABPB1 |
| ZNF610 | CSDC2 | ZNF8 | CERS3 | ZNF189 |
| CREB3L2 | POU6F1 | ATF6B | ZEB1 | TP63 |
| ZNF350 | ZNF317 | SP100 | HOXD10 | TCF12 |
| HOXA6 | IGHM | LMX1B | RXRB | LCOR |
| ZNF285 | ETV1 | ZNF653 | E2F8 | ESRRG |
| LARP4 | KLF12 | TSC22D1 | ZNF276 | GTF2H2 |
| PAX3 | ZIK1 | ZFYVE20 | SMAD1 | MYF6 |
| TEAD4 | MRPS31 | LARP1B | PPP2R3B | PLEK |
| ZNF317 | ZNF43 | IRF6 | ZNF550 | HOXA3 |
| ZNF783 | FOXA3 | ZNF543 | GCM1 | ZC3H14 |
| PRB4 | DMRTC2 | ZNF22 | HOGA1 | ZNF695 |
| TBR1 | XPA | ZNF26 | ZNF345 | PPP2R3B |
| TFR2 | ZNF32 | CUL5 | ID1 | PHB |
| SMAD9 | MBNL1 | ZKSCAN5 | ZNF2 | ZFHX3 |
| ZNF544 | FOXP4 | RBM10 | COPS2 | ETV5 |
| ZNF513 | RAPGEF3 | HOXB5 | ZNF786 | EWSR1 |
| AEBP2 | ZNF323 | ZNF446 | ZNF223 | ZNF195 |
| ZNF140 | ZNF490 | NR2E3 | GABPB1 | ATF1 |
| CPSF4 | ZNF558 | ZNF710 | ZNF276 | RPA2 |
| TUT1 | SCML4 | ZNF622 | NFYB | ZNF2 |
| CREB1 | PRDM4 | MYNN | ZNF416 | HINFP |
| REPIN1 | GATA1 | ZNF398 | ZNF672 | APTX |
| ZC3H8 | THAP6 | DPF3 | MIER1 | KLF9 |
| ZNF460 | STAT5B | PRDM5 | ZSCAN5A | C9ORF78 |
| ZNF571 | ONECUT2 | PRKRIR | RPA4 | ZBTB10 |
| ZDHHC19 | HAND1 | COPS3 | H1F0 | ZNF24 |
| ZNF334 | NR1H2 | HMGB4 | DEPDC7 | KLF4 |
| ZNF34 | MYOG | RAB11FIP3 | GABPB1 | HLA-DQB2 |
| ZNF71 | MEF2B | ZNF524 | RNF213 | RNF114 |
| ACY1 | IGHM | TUT1 | ZNF320 | ZNF10 |
| RBM10 | ZNF280A | TCF4 | STAT4 | ZGPAT |
| DACH2 | OLIG3 | HSF1 | RFX4 | PSMD12 |
| FOXN4 | JUNB | RLF | ZNF572 | ZNF430 |
| GTF2H2 | U2AF1 | ZNF511 | LARP1 | ZNF114 |
| SMARCE1 | TBX3 | ERG | EBF1 | LARP1 |
| SSRP1 | COPS4 | E4F1 | DEPTOR | ZBED1 |
| FOSL1 | HEY2 | ZNF561 | ZNF585A | NFE2L1 |
| ASCL2 | ZNF576 | AKAP8 | BARX1 | SIX1 |
| PPARG | NR1D1 | TAL2 | ZNF213 | DPF1 |
| THAP11 | ZBTB45 | ZC3H10 | ZNF232 | MRPS17 |
| ARID3A | ZNF264 | ZNF20 | POLR2L | ZDHHC11 |
| HOXB9 | SP4 | RNF166 | ZFP91 | PSMD12 |
| RARB | MXI1 | BCL6B | MRPL2 | POU2F1 |
| GATAD2A | TSC22D4 | ZSCAN29 | ZMAT5 | ZNF227 |
| ALX1 | ATF2 | DEPDC1B | GTF2F2 | HMG20B |
| IGHM | ZNF577 | ZNF200 | FOXA1 | STAT5A |
| ZNF124 | ZNF18 | RFX4 | HMG20A | ZBTB24 |
| DMC1 | PRB3 | ZNF454 | EGR1 | TLX3 |
| ZNF169 | NOC4L | CBX2 | KLF6 | ZNF501 |
| SP3 | TCF4 | ZNF207 | SMAD3 | ZNF331 |
| RELA | ZBTB9 | GRHL3 | MIXL1 | IRF5 |
| FOS | SNAI2 | TOE1 | EWSR1 | ZBP1 |

In an aspect, client proteins can participate in cell motility, cytotoxicity, metastasis, survival, organ destruction, phosphorylation of HSP90beta, covalent modifications of chaperone proteins and/or their clients. In an aspect, a client protein can be an agent of interest. For example, HSP90 can be HSP90α or HSP90β. In an aspect, HSP90 can be HSP90β. In an aspect, an altered phosphorylation of HSP90 can be a decrease in phosphorylation of serine-226 of HSP90β. For example, the decrease in phosphorylation of serine-226 of HSP90β can be a decrease relative to no chemical control.

In an aspect, the method can further comprise assaying one or more agents of interest for stabilizing a HSP90β/CDC37 heterocomplex, and identifying and selecting one or more agents of interest stabilizing the HSP90β/CDC37 heterocomplex. For example, in an aspect, stabilization of the HSP90β/CDC37 heterocomplex can comprise stabilization to proteolytic degradation, preserving intact polypeptide or reducing proteolytic degradation products.

In an aspect, the method can further comprise assaying one or more agents of interest for changes in signature of client proteins bound to a HSP90β/CDC37 heterocomplex, and identifying and selecting one or more agents of interest reducing or inhibiting association of the HSP90β/CDC37 heterocomplex and kinases participating in cell motility. In an aspect, the one or more agents of interest can alter the signature of client proteins bound to the HSP90β/CDC37 heterocomplex by reducing or inhibiting association of the HSP90β/CDC37 heterocomplex and a subset of client proteins. In an aspect, the subset of client proteins are or comprise one or more kinases participating in cell motility.

In an aspect, the method can further comprise assaying one or more agents of interest for stabilizing the chaperone-co-chaperone complex, wherein the chaperone-co-chaperone complex is HSP90β/CDC37, and identifying and selecting one or more agents of interest stabilizing the chaperone-co-chaperone complex. For example, in an aspect, stabilizing the HSP90β/CDC37 can comprise stabilizing proteolytic degradation, preserving intact polypeptide or reducing proteolytic degradation products.

In an aspect, the method can further comprise assaying one or more agents of interest for changes in signature of client proteins bound to the chaperone-co-chaperone complex, wherein the chaperone-co-chaperone complex is HSP90β/CDC37, and identifying and selecting one or more agents of interest changing the signature of client proteins bound to the chaperone-co-chaperone complex. In an aspect, the one or more agents of interest can alter the signature of client proteins bound to HSP90β/CDC37 by reducing or inhibiting association of HSP90β/CDC37 to a subset of client proteins. In an aspect, the subset of client proteins can be one or more kinases or can comprise one or more kinases participating in cell motility.

Examples of kinases participating in cell motility include, but are not limited to, RAF1, RIPK1, SGK3, MAP3K15, NTRK1, MAP3K6, GSG2, RIPK2, NEK2, PRKCB1, LIMK1, TGFBR1, LOC340371, PRKACG, CAMK2B, LOC91461, and NLK.

Disclosed herein are methods of evaluating or monitoring the effectiveness of a cancer treatment in a subject having cancer or diagnosing cancer in a subject suspected of having cancer. The methods can comprise measuring the level or activity of at least one biomarker in a sample comprising Raf1. In an aspect, the method can measure Raf1 phosphorylation in sample. In an aspect, the phosphorylation status of Raf1 in a sample can indicate the effectiveness of an agent of interest. In an aspect, the phosphorylation status of Raf1 in a sample can indicate the effectiveness of a cancer treatment in a subject. In an aspect, dephosphorylation or inhibition of Raf1 phosphorylation in a sample can indicate that an agent of interest has anti-cancer activity. In an aspect, dephosphorylation or inhibition of Raf1 phosphorylation in a sample can indicate that that a particular cancer treatment can be effective in reducing or ameliorating one or more signs of cancer.

Methods of Treatment

Also disclosed herein are methods of inhibiting, preventing or treating cancer or metastatic cancer in a subject. In an aspect, the method can comprise administering to the subject a therapeutically effective amount of an agent of interest identified by any of methods disclosed herein or a salt or a derivative thereof, thereby inhibiting, preventing or treating cancer or metastatic cancer in the subject.

Disclosed herein are methods of treating cancer or metastatic cancer in a subject. In an aspect, the method can comprise: identifying a subject in need of treatment; and administering a therapeutically effective amount of the agent of interest identified by the method disclosed herein or a salt or a derivative thereof.

Disclosed herein are methods of inhibiting cancer or metastatic cancer in a subject. In an aspect, the method can comprise: identifying a subject in need of treatment; and administering a therapeutically effective amount of the agent of interest identified by the method disclosed herein or a salt or a derivative thereof.

Disclosed herein are methods of preventing cancer or metastatic cancer in a subject. In an aspect, the method can comprise: identifying a subject in need of treatment; and administering a therapeutically effective amount of an agent of interest identified by the method disclosed herein or a salt or a derivative thereof.

The compositions described herein can be formulated to include a therapeutically effective amount of any of the agents of interest identified using any of the methods disclosed herein described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The compositions described herein can be formulated in a variety of combinations. The particular combination of one or more of the agents of interest identified in any of the methods disclosed herein can vary according to many factors, for example, the particular the type and severity of the cancer.

The compositions described herein can be administered to the subject (e.g., a human patient) in an amount sufficient to delay, reduce, or preferably prevent the onset of clinical disease. Accordingly, in some aspects, the subject can be a human subject. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

In some aspects, the cancer can be a primary or secondary tumor. In an aspect, the cancer can be a metastatic tumor. In other aspects, the primary or secondary tumor is within the patient's breast, lung, lung, prostate, head or neck, brain, bone, blood, colon, gastrointestinal track, esophagus or liver. In yet other aspects, the cancer has metastasized. In some aspects, the cancer may metastasize to one or more of the following sites: the breast, lung, liver or bone.

Disclosed herein, are methods of treating a patient with cancer. The cancer can be any cancer. In some aspects, the cancer can be breast cancer, lung cancer, brain cancer, liver cancer, prostate cancer, head or neck cancer, a blood cancer, colon cancer, gastrointestinal track cancer, bone cancer or esophageal cancer. In an aspect, the subject has been diagnosed with cancer prior to the administering step.

The therapeutically effective amount or dosage of the any of the agents of interest identified in any of the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The present compounds can be given to prior to, simultaneously with or after treatment with other agents or regimes.

Pharmaceutical Compositions

As disclosed herein, are pharmaceutical compositions, comprising one or more of the therapeutic compositions disclosed herein. As disclosed herein, are pharmaceutical compositions, comprising any of the agents of interest identified in any of the methods disclosed herein and a pharmaceutical acceptable carrier described herein. In some aspects, the composition can be formulated for oral or parental administration. In an aspect, the parental administration can be intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the agents of interest identified using any of the methods disclosed herein dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

Kits

In an aspect, kits are provided for measuring binding or activity of a client protein to a chaperone-co-chaperone complex disclosed herein. In an aspect, kits are provided for measuring the one or more biomarkers disclosed herein. The kits can comprise materials and reagents that can be used for measuring the level or activity of the one or more client proteins and/or the one or more biomarkers. These kits can include the reagents needed to carry out the measurements of the binding, activity or level of the client protein and/or biomarkers. Alternatively, the kits can further comprise additional materials and reagents.

The following examples are presented to illustrate the present invention and to assist one of ordinary skill in making and using the same. The examples are not intended in any way to otherwise limit the scope of the invention.

### EXAMPLES

### Example 1: KBUO46 does not Inhibit Kinase or Phosphatase Activity

Kinase assay system #1. The KINOMEscan™ assay (Ambit Biosciences). This assay evaluates 442 kinases, including 400 distinct parental kinases plus mutants known to alter activity or responsiveness. It does so in the context of an assay that measures the ability of putative inhibitors to inhibit binding of bacterial purified kinase to immobilized phospho-acceptor protein substrate. This approach has been successfully to identify kinase interactions of several small molecule kinase inhibitors (Fabian, M. A. et al., Nat Biotechnol 23, 329-36 (2005); and Karaman, M. W. et al., Nat Biotechnol 26, 127-32 (2008)). KBU2046 was tested at 10 μM. This assay was completely negative. There were two initial false positives (for a false positive rate of 0.4%), that were subsequently found to be negative in a dedicated follow up analysis. Specific and important negative findings include MKK4, p38 MAPK (α, β, γ and δ isoforms) and RAF1. Conclusions: there was no evidence that KBU2046 inhibits kinase function by competing for phospho-acceptor binding.

Kinase assay system #2. The Kinex™ kinase assay system (Kinexus Proteomics Company). This platform uses recombinant human protein kinases expressed in an insect expression system, thus allowing an avenue for post-translational modification. Also, this platform measures inhibition of ATP binding. This platform putatively measured 200 different kinases, and we tested KBU2046 at 1 and 10 μM. This assay was also completely negative. There were two important technical issues. First, at 10 μM KBU2046 interfered with the colorimetric-based readout of the assay. Second, it was found that ⅓^{rd} of the control enzymes were not active. However, while considering the above factors, this screen was informative for a number of kinases (where controls were active and at KBU2046 concentrations that did not interfere with detection). In this regard, it is highlighted that KBU2046 did not inhibit p38 MAPK (all isoforms), MKK4 or RAF1. Conclusions: there was no evidence that KBU2046 inhibits kinase function by competing for ATP binding in the active site.

Kinase assay system #3. KinaseProfiler™ and PhosphataseProfiler™ assay platforms (Millipore). This platform is radiometric-based (considered gold standard). It measures competition with respect to ATP for kinases and substrate protein for phosphatases. Most proteins were expressed via an insect-based system, and it evaluates a panel of 284 kinases and 20 phosphatases. There were two initial false positives, for a false positive rate of 0.7%, but both failed to be confirmed upon in-depth investigation. Important negative findings include: MKK4, MKK6, p38 MAPK, MAPKAPK2, RAF1, ERK, MEK1, JNK1, 2 and 3, and numerous other MAPK cascade-associated kinases. Conclusions: there is no evidence that KBU2046 inhibits kinase activity by competing for ATP binding in the active site. No evidence supports inhibition of phosphatase activity.

### Example 2: Identifying a Selective Inhibitor of Cell Motility

Flavonoids were selected as a chemical scaffold to advance probe synthesis because they exert a wide range of biological effects (Andersen et al., (CRC Press, Boca Raton, 2006)). 4′,5,7-trihydroxyisoflavone (genistein) was the starting point because of its known anti-motility properties. It was previously demonstrated that nanomolar concentrations of genistein inhibit human prostate cancer (PCa) cell invasion in vitro (Huang, X. et al., Cancer Research 65, 3470-8 (2005)), metastasis in a murine orthotopic model (Lakshman, M. et al., Cancer Res 68, 2024-32 (2008)) and in the context of a prospective human trial that it downregulates matrix metalloproteinase 2 (MMP-2) expression in prostate tissue (Xu, L. et al., Journal of the National Cancer Institute 101, 1141-1155 (2009)). While its diverse spectrum of biological effects renders it unusable as a selective and potent biological probe, these same properties maximize its potential to selectively probe a wide spectrum of bioactive sites upon chemical diversification.

A series of related molecular probes were developed through phenotypically driven structure activity relationship studies, specifically through chemical modification of the genistein structure (aromatic substitution and ring saturation). These compounds were advanced by iterative selection for inhibition of human PCa cell invasion (FIG. 1a and FIGS. 8-9). A parallel goal was deselection for inhibition of cell growth (an indicator of off-target effects). Knowing that genistein has estrogenic action, and guided by the crystal structure of genistein bound to estrogen receptor (ER) (Manas, E. S. et al., Structure 12, 2197-207 (2004), another goal was deselection for ER-binding. Through this strategy, (±)-3(4-fluorophenyl)chroman-4-one (KBU2046), a halogen-substituted isoflavanone, was discovered (FIG. 1a).

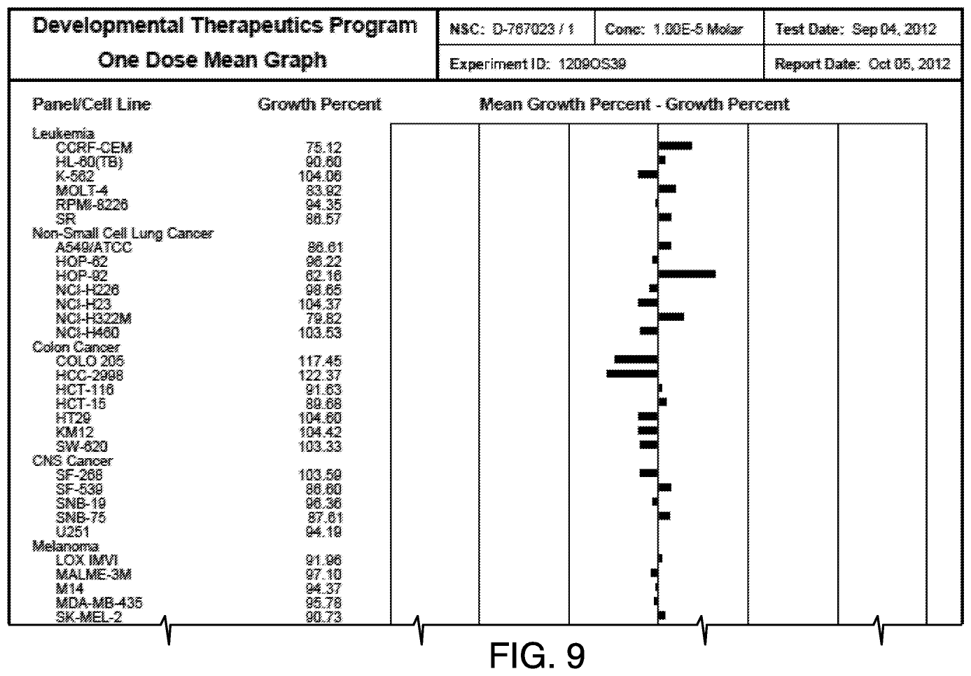

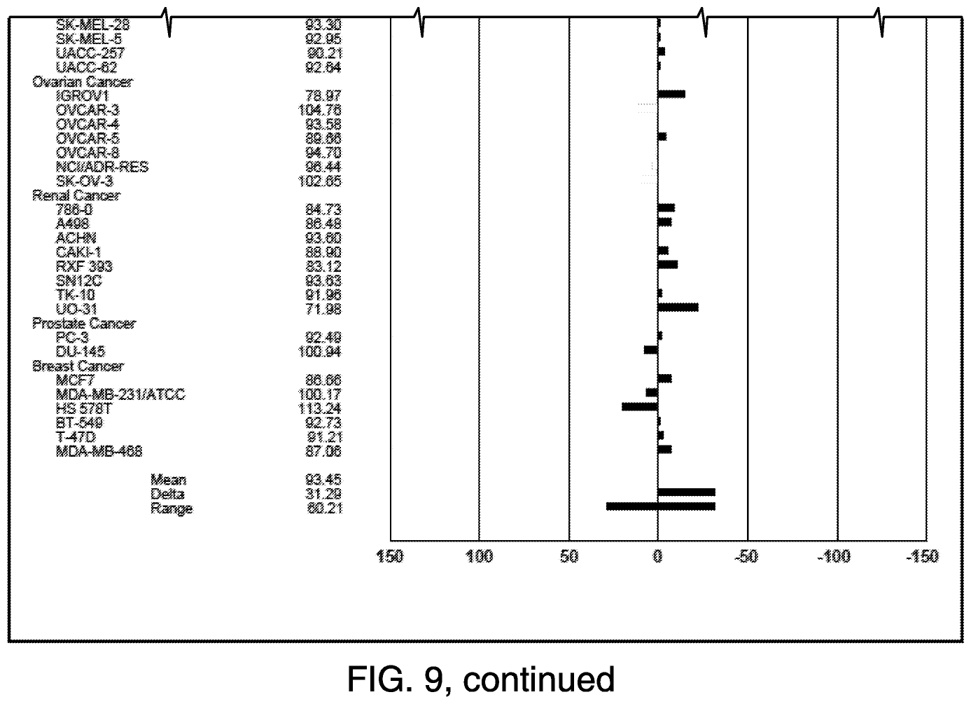

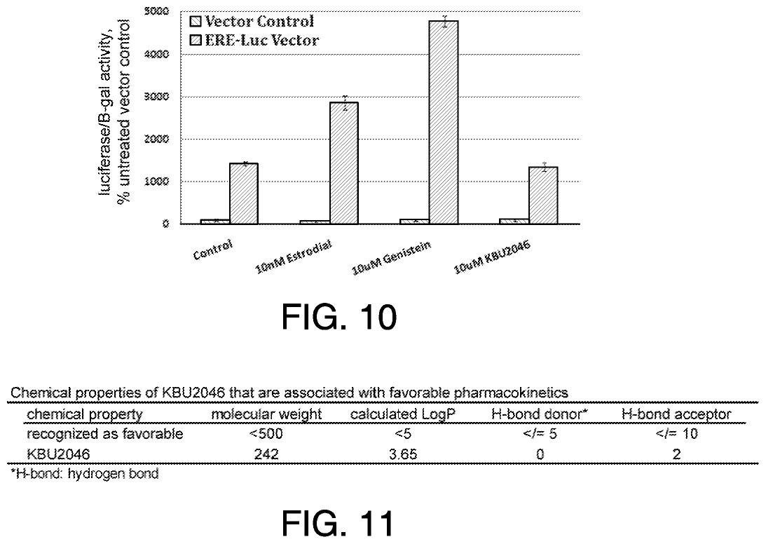

KBU2046 inhibits cell invasion with efficacy equal-to-or-greater-than that of genistein for human prostate cells, including normal prostate epithelial cells, as well as primary and metastatic PCa cells (FIG. 1b). Cell migration is a major determinant of cell invasion (Friedl et al., Nat Rev Cancer 3, 362-74 (2003)) and KBU2046 inhibited the migration of human prostate, breast, colon and lung cancer cells (FIG. 1c). Importantly, KBU2046 had high selectivity in cellular assays. It was not toxic to human prostate cells (FIG. 1d), to human bone marrow stem cells (FIG. 1e), nor to cells in the NCI-60 cell line panel (FIG. 9). Bone marrow toxicity is induced by a wide spectrum of therapeutic agents, and is frequently a dose-limiting toxicity for anti-cancer agents. Furthermore, in estrogen-responsive human breast cancer MCF-7 cells, KBU2046 did not activate estrogen-responsive genes (FIG. 1f and FIG. 10).

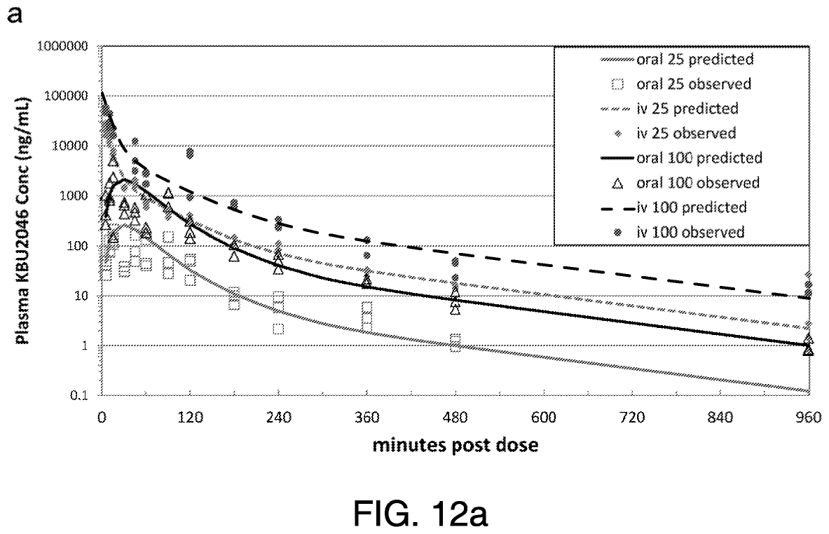

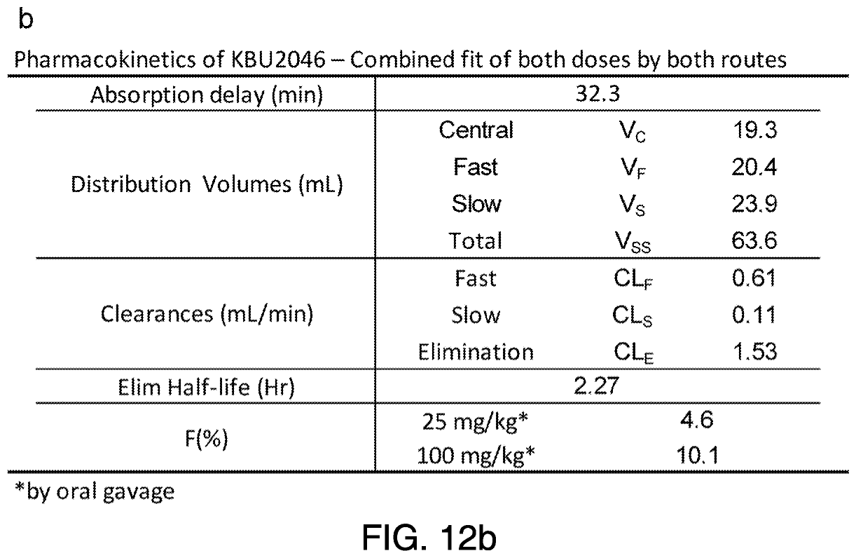

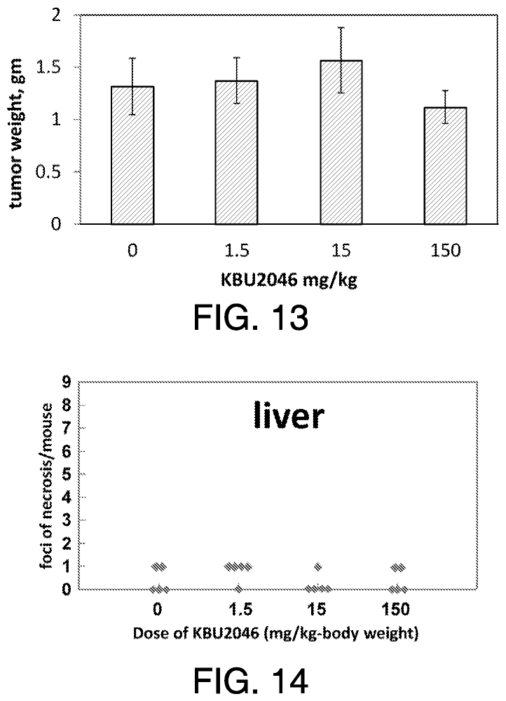

KBU2046 Inhibits Metastasis and Prolongs Survival. Because metastasis is a systemic process, effective small molecule probes must operate at the systemic level. The probe was designed to contain chemical properties known to be associated with systemically active small molecules (FIG. 11). Employing a murine orthotopic implantation model of human PCa previously characterized⁸, KBU2046 was shown to significantly decrease metastasis in a dose-dependent manner by up to 92%, at plasma concentrations of 1.1-24 nM (FIGS. 2a-b). Comprehensive characterization of KBU2046 pharmacokinetics demonstrated maintenance of plasma concentrations >24 nM for 9.3 hours after a single oral dose, and allowed for characterization of pharmacokinetic parameters (FIG. 2c and FIG. 12). At the systemic level, KBU2046 was a highly selective inhibitor of metastasis. Comprehensive analysis of primary tumor growth, animal behavior, weight, histologic examination of multiple organs and serum chemistry profiling, failed to identify KBU2046-associated off-target effects (FIGS. 13-14).

Recognizing the established link between metastasis and decreased survival in humans, KBU2046's impact on survival was evaluated. The orthotopic PCa model exhibits tumor growth around the urogenital tract, inhibiting renal function and precluding assessment of the impact of metastatic burden on survival. However, orthotopic implantation of human breast cancer cells, followed by surgical removal of the resultant primary tumor, provides a murine model wherein survival is dictated by metastatic burden (du Manoir, J. M. et al., Clin Cancer Res 12, 904-16 (2006)). KBU2046 significantly prolonged the survival of mice treated in a post-surgery adjuvant setting (FIG. 2d).

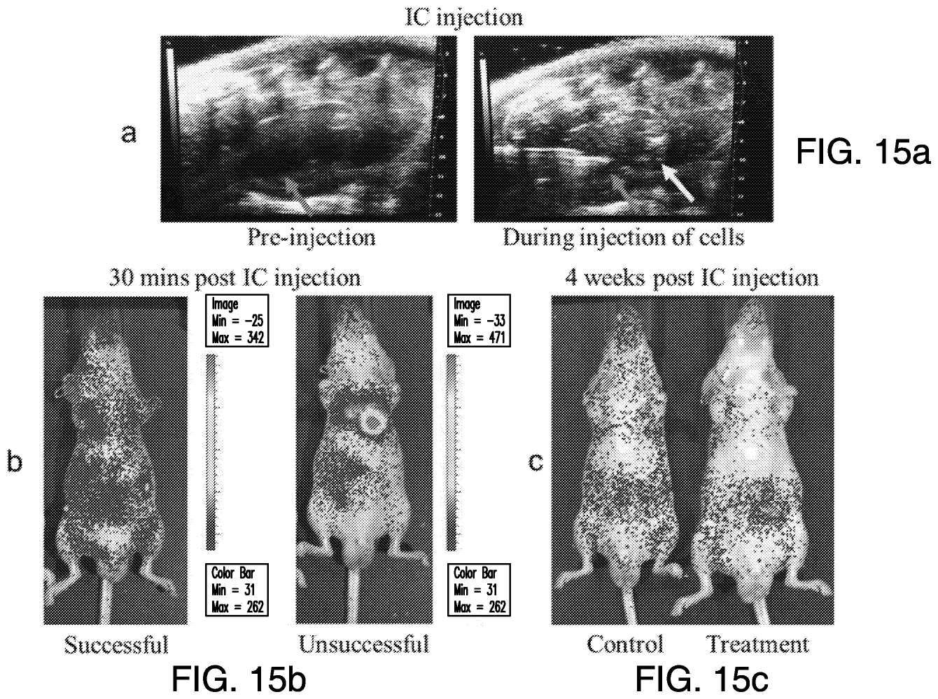

If KBU2046 were inhibiting metastasis through inhibition of cell motility, then it should have little-to-no effect upon the metastatic process once cells have implanted into distant organs. Recognizing that skeletal metastases are a major clinical problem with PCa, further assessment of this paradigm was pursued with an established PCa bone metastasis model (Chu, K. et al., Mol Cancer Res 6, 1259-67 (2008)). PC3 luciferase tagged (PC3-luc) cells were delivered by ultrasound-guided intracardiac (IC) injection and metastatic outgrowth monitored weekly via IVIS imaging (FIG. 3a and FIG. 15). Compared to control mice, the Pre cohort of mice (KBU2046 treatment starts 3 days prior to IC injection and continuing through the end of the experiment) experienced a significant decrease in total metastatic burden, as well as decreased metastasis to the mandible (for which this model is designed) (FIGS. 3b-c). In contrast, with the Post cohort of mice, metastasis to the total body as well as to the jaw do not differ from control mice. With the Post cohort of mice, cells are given 3 days post-IC injection to invade into distant organ sites before treatment is begun, with treatment then continuing through the end of the experiment. In the Pre7Stop cohort of mice, treatment starts 3 days prior to IC injection, continues through day 7 post-IC injection and is not given for the remaining 3 weeks of the experiment. Findings in this cohort of mice suggest an intermediate outcome between that of Pre and Post cohorts. Specifically, total body metastasis is significantly decreased in the Pre7Stop cohort, compared to both control and Post cohorts. While jaw metastasis is significantly decreased compared to control, it is not significantly decreased compared to the Post cohort of mice, yet the average value is below that of Post mice and is approaching that of the Pre cohort of mice. Degradation of the mandible was quantified with Computed Tomography in Pre, Post and control cohorts, demonstrating decreased destruction of bone in the Pre cohort of animals (FIGS. 3d-e).

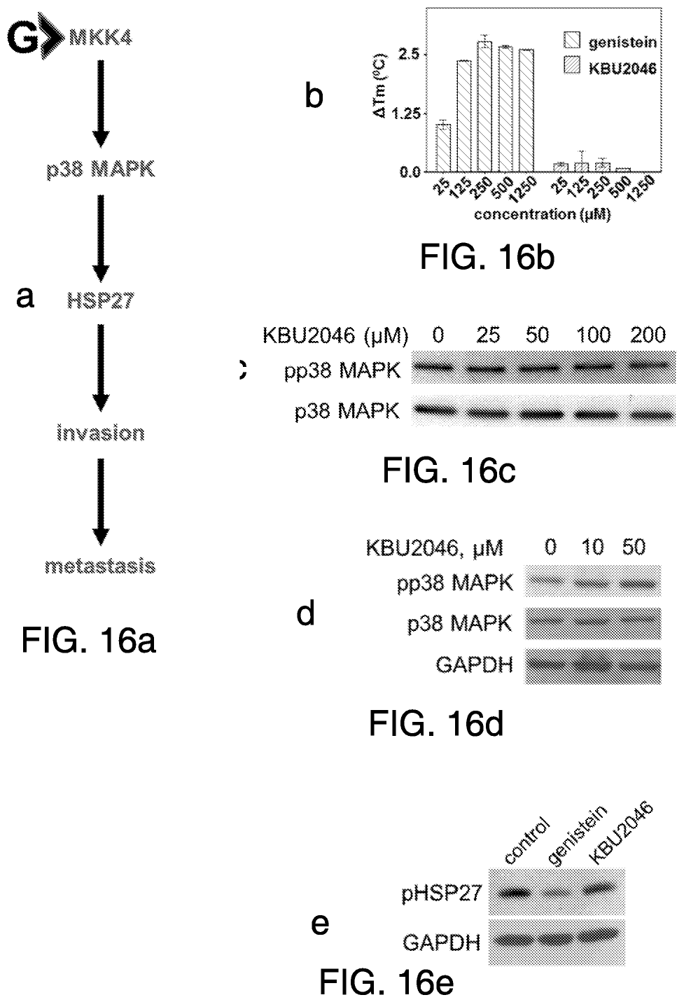

KBU2046 induces changes in HSP90β phosphorylation. With the aforementioned positive phenotypic cellular and animal studies, the molecular basis for KBU2046's biological action was sought. The initial investigations were guided by prior demonstration that low nanomolar concentrations of genistein inhibited the kinase activity of mitogen-activated protein kinase 4 (MKK4/MAP2K4/MEK4), (Xu, L. et al., Journal of the National Cancer Institute 101, 1141-1155 (2009)), in turn inhibiting downstream phosphorylation of p38 MAPK (Huang, X. et al., Cancer Research 65, 3470-8 (2005)) and of heat shock protein 27 (HSP27), (Xu, L. et al., Mol Pharmacol 70, 869-77 (2006)). This translated into inhibition of MMP-2 expression and cell invasion in vitro, inhibition of human PCa metastasis in mice (Lakshman, M. et al., Cancer Res 68, 2024-32 (2008)) and decreased MMP-2 expression in human prostate tissue (Xu, L. et al., Journal of the National Cancer Institute 101, 1141-1155 (2009)). In contrast to genistein, KBU2046 did not bind to MKK4 nor inhibit its kinase activity in vitro, and it did not inhibit downstream phosphorylation of p38 MAPK or of HSP27 in cells (FIG. 16). This finding, while surprising, demonstrates that the chemical probe strategy de-selected for inhibition of the MKK4 signaling axis. Importantly, this provides a measure of the unbiased nature of the chemical probe strategy.

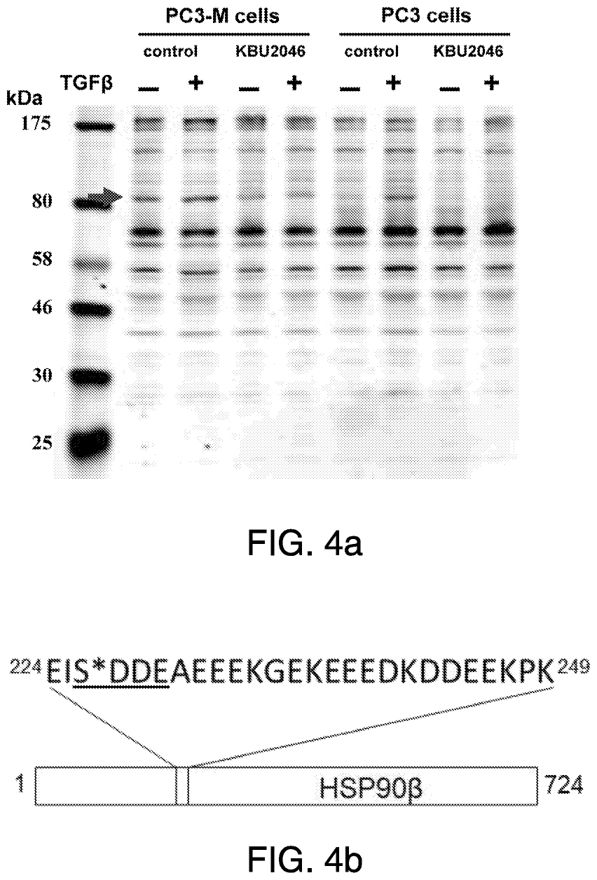

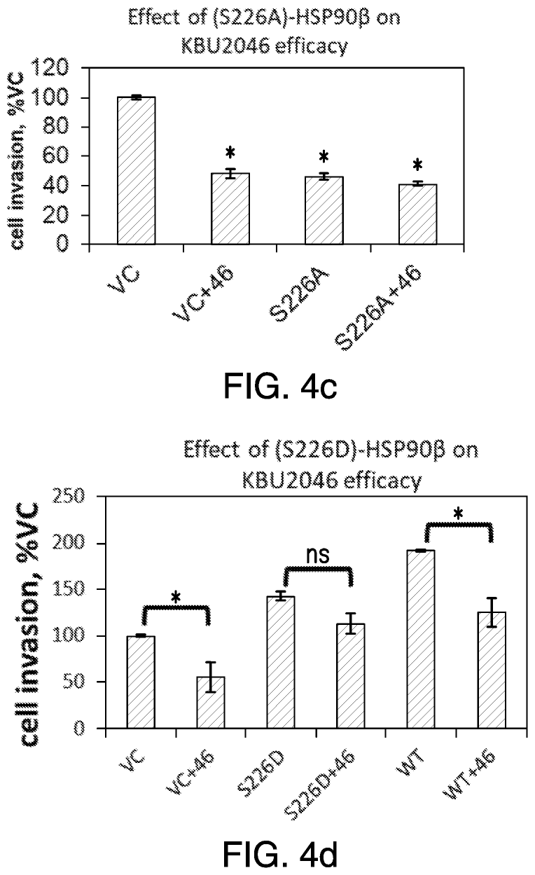

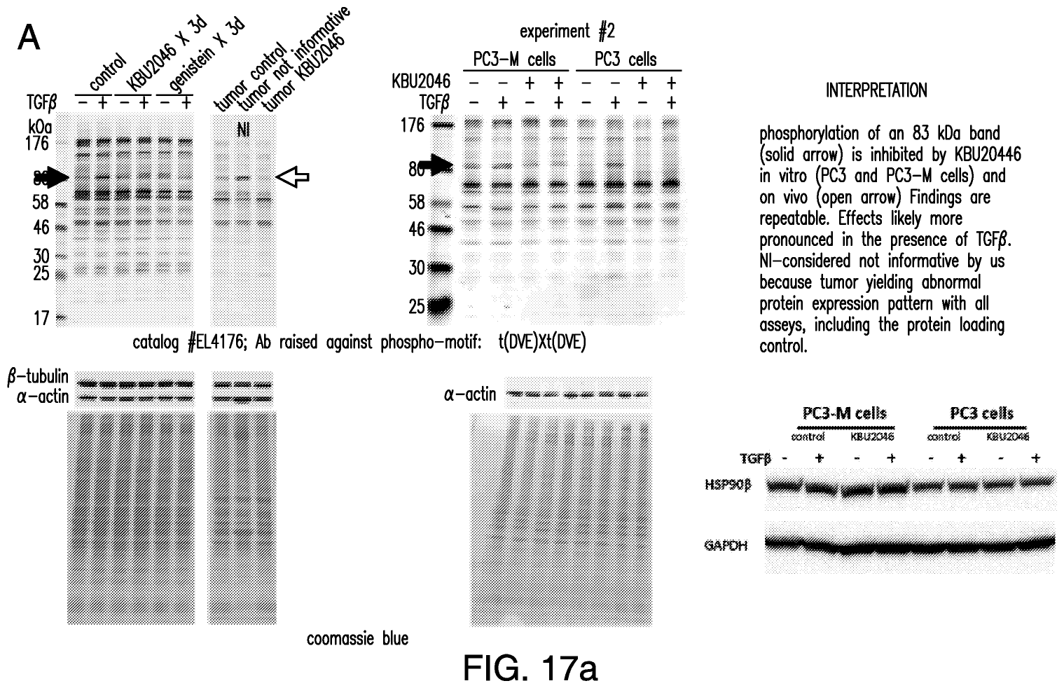

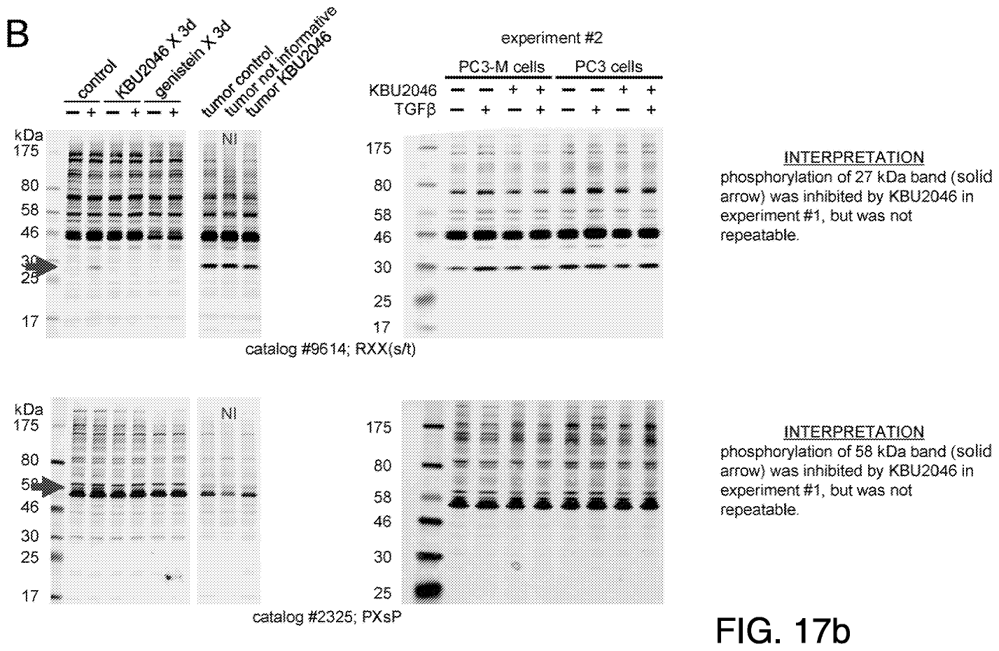

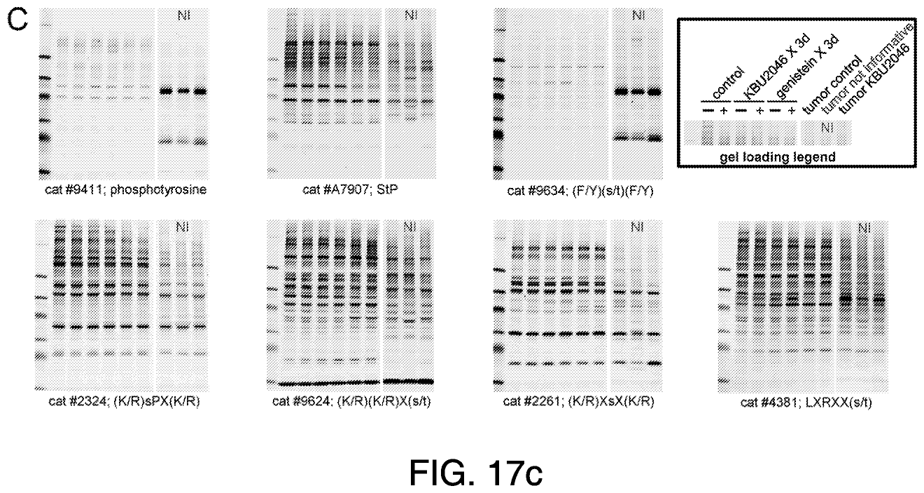

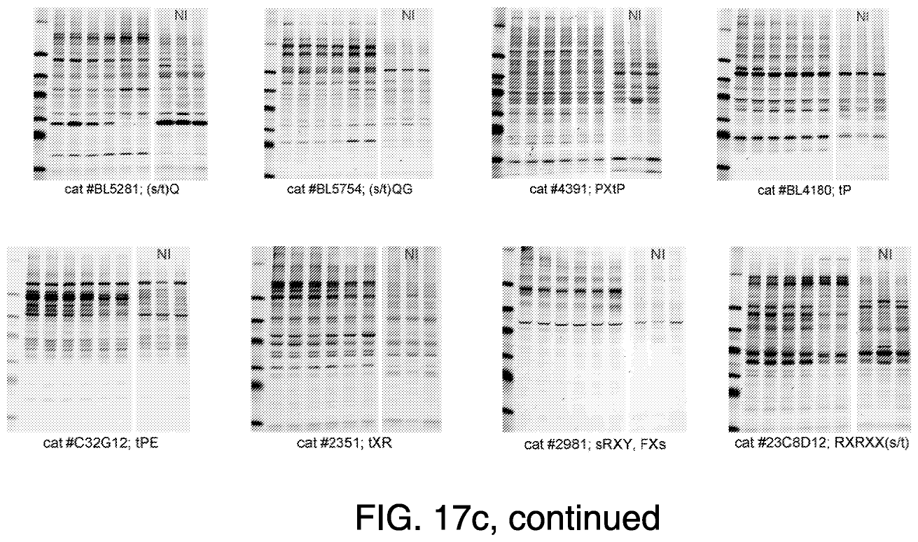

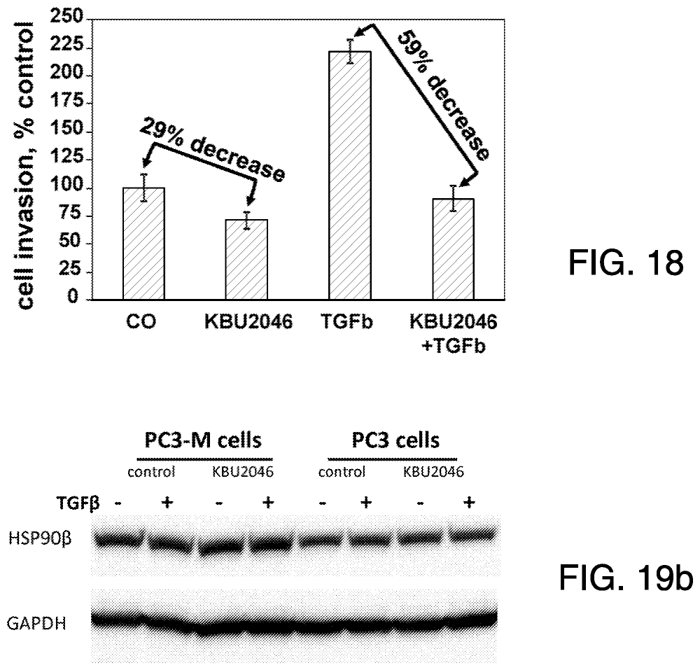

Seeking to identify KBU2046's biological target(s), alternative methods were pursued. The KinomeView® panel of antibodies (Cell Signaling Technology, Inc.) detect established protein phosphorylation motifs, and were used to probe for KBU2046-induced changes in protein phosphorylation (FIG. 4a and FIG. 17). Phosphoprotein changes that met the following criteria were prioritized: were induced in cells in vitro as well as in tumors of treated mice (from FIG. 2a), that counteracted transforming growth factor β (TGFβ)-induced effects, and that were reproducible. TGFβ is ubiquitous in vivo, is known to increase PCa cell invasion (Huang, X. et al., Cancer Research 65, 3470-8 (2005)), and KBU2046's anti-invasion efficacy remains in spite of TGFβ-stimulated increases in cell invasion (FIG. 18). Genistein was evaluated under identical treatment conditions for comparison. Genistein's many pharmacologic effects induced widespread changes in protein phosphorylation (FIG. 17). In contrast, KBU2046 induced a single change that met the pre-specified criteria, i.e., a decrease in intensity of an 83 kDa protein band (blue arrow in FIG. 4a). In tumors of treated animals KBU2046 had this same effect on this 83 kDa protein band (green arrow in FIG. 17a). The high molecular selectivity of KBU2046 was further supported by its failure to inhibit over 400 different protein kinases and 20 phosphatases examined, in three different in vitro assay systems (see, Example 1).

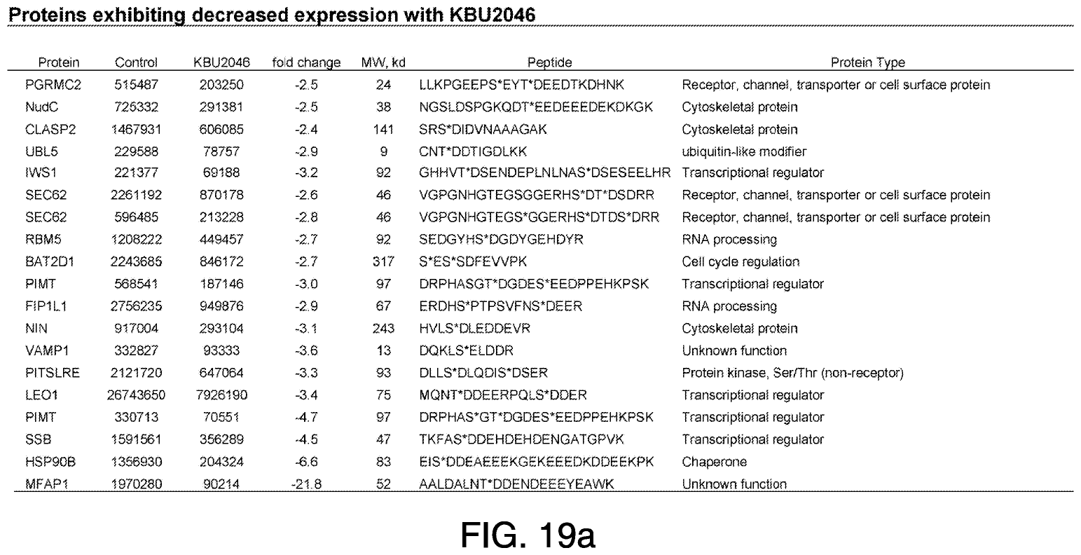

The 83 kDa protein was identified by pretreating PC3 cells with KBU2046 or vehicle control, treating with TGFβ and performing LC-MS/MS analysis on proteins pulled down by the KinomeView® antibody used in FIG. 4a. Resultant data were analyzed with the SEQUEST/Sorcerer data analysis suite, and proteins further selected based upon predetermined parameters (FIG. 19). This approach yielded a single protein, HSP90β, and indicated that KBU2046 decreased the abundance of phosphorylated Ser²²⁶ on HSP90β by 6.6 fold (FIG. 4b and FIG. 19).

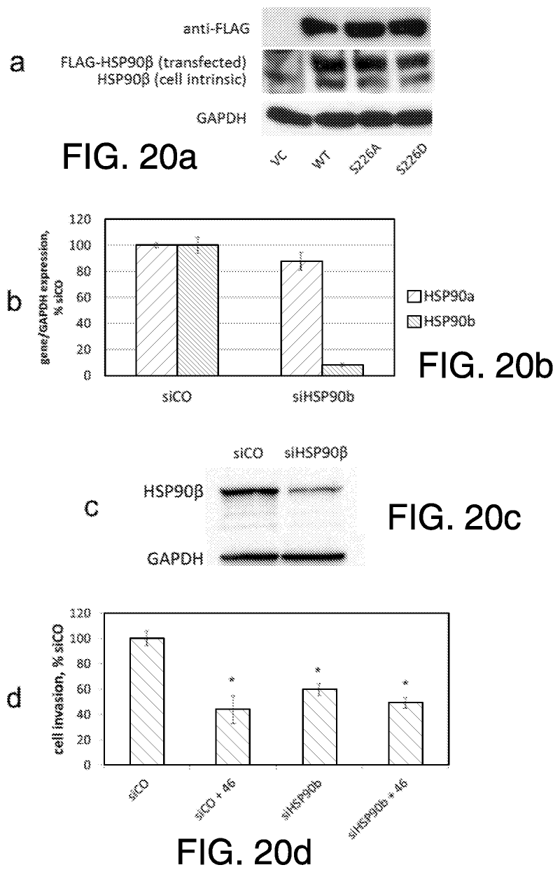

The (S226A)-HSP90β construct lacks a Ser²²⁶ residue, precluding phosphorylation at that site, represents a constitutive inactive mimic, and mimics the effect of KBU246 on that residue (i.e., dephosphorylation). As expected, transfection of cells with (S226A)-HSP90β inhibited cell invasion, compared to vector control (VC) transfected cells. Further, if KBU2046 were exerting efficacy by inhibiting phosphorylation of the Ser²²⁶ residue, then removal of that residue should, by definition, preclude additional efficacy. This is exactly what is observed: in (S226A)-HSP90β transfected cells, KBU2046 does not further inhibit cell invasion, while it significantly inhibits invasion in VC cells (FIG. 4c and FIG. 20). The selectivity of HSP90β in mediating KBU2046 efficacy was further supported by demonstrating that siRNA-mediated HSP90β knockdown inhibited cell invasion and abrogated KBU2046 efficacy (FIG. 20). HSP90β-specific siRNA did not knockdown HSP90α (FIG. 20). Conversely, the pseudophosphorylated (S226D)-HSP90β construct contains a residue that provides a biological mimic of phosphorylated Ser²²⁶, and as such constitutes a constitutively active mutant. As expected, cells transfected with (S226D)-HSP90β were more invasive than VC cells. Recognizing that pseudophosphorylated constructs serve to mimic activated wild type protein, it was not surprising that (S226D)-HSP90β cells were not as invasive as WT-HSP90β cells. More importantly, if KBU2046 were exerting efficacy by inhibiting phosphorylation of the Ser²²⁶ residue, then the presence of a phospho-mimic residue should, by definition, decrease additional efficacy. This is exactly what is observed: in (S226D)-HSP90β transfected cells, KBU2046 did not significantly inhibit cell invasion, while it significantly inhibited invasion in both VC and WT-HSP90β cells (FIG. 4d and FIG. 20). These findings demonstrate that changes in the phosphorylation status of Ser²²⁶ on HSP90β can be altered by a small molecule, and appear to be associated with selective inhibition of cancer cell motility by KBU2046.

KBU2046 Selectively Disrupts Heterocomplex Function. KBU2046's effect upon HSP90β function is completely different from that of classical HSP90 inhibitors. The latter induce cytotoxicity and work by binding directly to HSP90, thereby inhibiting its enzyme activity, in turn affecting the function of large numbers of cellular kinases and other client proteins (Neckers, L. et al., Clin Cancer Res 18, 64-76 (2012)). In contrast, KBU2046 was not cytotoxic and its effects on protein phosphorylation were highly specific, demonstrating a lack of effects on kinase function. HSP90β is part of a large multiprotein chaperone complex whose function involves binding a large but specific set of regulatory proteins. It was reasoned that KBU2046 was changing the signature of bound client proteins, that the change was highly selective in terms of number of affected proteins, and that it was highly specific for proteins that regulate cell motility.

CDC37 is a co-chaperone that mediates the binding of over 350 client proteins to HSP90β, including over 190 kinases (Taipale, M. et al., Cell 150, 987-1001 (2012)). CDC37 is a flexible arm-like structure (protein data bank (PDB) ID: 2WOG), is highly dynamic (Vaughan, C. K. et al., Mol Cell 23, 697-707 (2006)), enables binding of large numbers of kinases, defines their positioning and thereby their potential to affect HSP90β phosphorylation status. It was reasoned that KBU2046 was binding to either CDC37 or HSP90β, that this altered the function of the CDC37/HSP90β heterocomplex resulting in a change in the spectrum of bound client kinase proteins, that changes were highly selective and that this altered binding spectrum would in turn be responsible for KBU2046's effects upon cell motility.

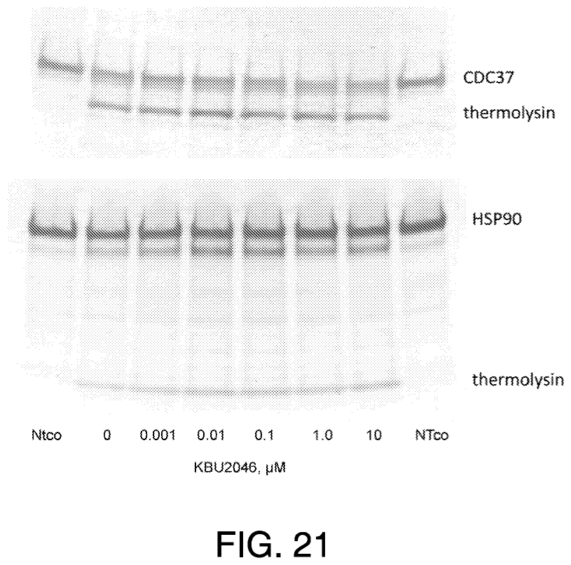

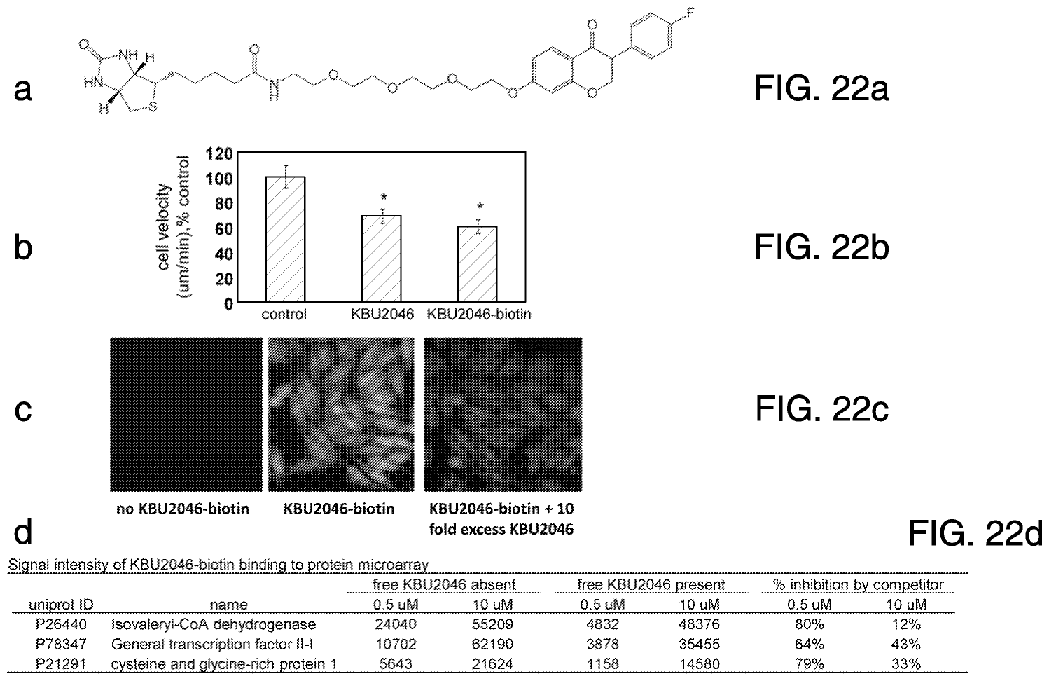

There was no evidence of KBU2046 binding to either CDC37 or HSP90β by biophysical methods, inclusive of isothermal titration calorimetry, fluorescence-based thermal shift assay, biolayer interferiometry or by dynamic light scattering, nor by the biochemical method of drug affinity responsive target stability (DARTS) assay (FIG. 21). DARTS provides a sensitive measure of ligand-induced changes in protein structure and dynamics by measuring the ability of a ligand to protect its target from protease digestion (Lomenick, B. et al., Proc Natl Acad Sci USA 106, 21984-9 (2009)). Although KBU2046 did not bind CDC37 or HSP90β individually, because CDC37 and HSP90β associate to form a heterocomplex (Vaughan, C. K. et al., Mol Cell 23, 697-707 (2006)), CDC37 and HSP90β were combined in a DARTS assay, demonstrating that KBU2046 protected both proteins from digestion (FIG. 5a). The intensity of the CDC37 band increased, that of the HSP90β degradation product decreased, and both effects were statistically significant, concentration-dependent and were evident at 10 nM. Further, the high selectivity of KBU2046 for protein binding was additionally supported by synthesizing a biotin chemical linker to KBU2046, demonstrating that it retained biological activity, that it bound to intact cells (i.e., under physiological conditions of CDC37/HSP90β heterocomplex formation), and that it failed to bind to an array of over 9,000 human proteins (FIG. 22). Together, these findings demonstrate that KBU2046 will not bind to either CDC37 or HSP90β, but that it will bind when both proteins are present and able to form heterocomplexes. Further, these findings also indicate that KBU2046 is not acting as a classical HSP90 inhibitor. Classical HSP90 inhibitors bind isolated HSP90, without the need for co-chaperones being present, are characterized by their cytotoxic effects, are systemically toxic, particularly to the liver, and broadly inhibit client kinase protein binding, thereby exerting widespread effects upon cellular signaling and affecting a wide array of cellular processes (Neckers, L. et al., Clin Cancer Res 18, 64-76 (2012)). In contrast, KBU2046 exhibits a complete lack of cellular cyctotoxicity and systemic toxicity, everts highly specific effects in both molecular-based protein phosphorylation and cellular-based functional assays and will not bind HSP90 in isolation.

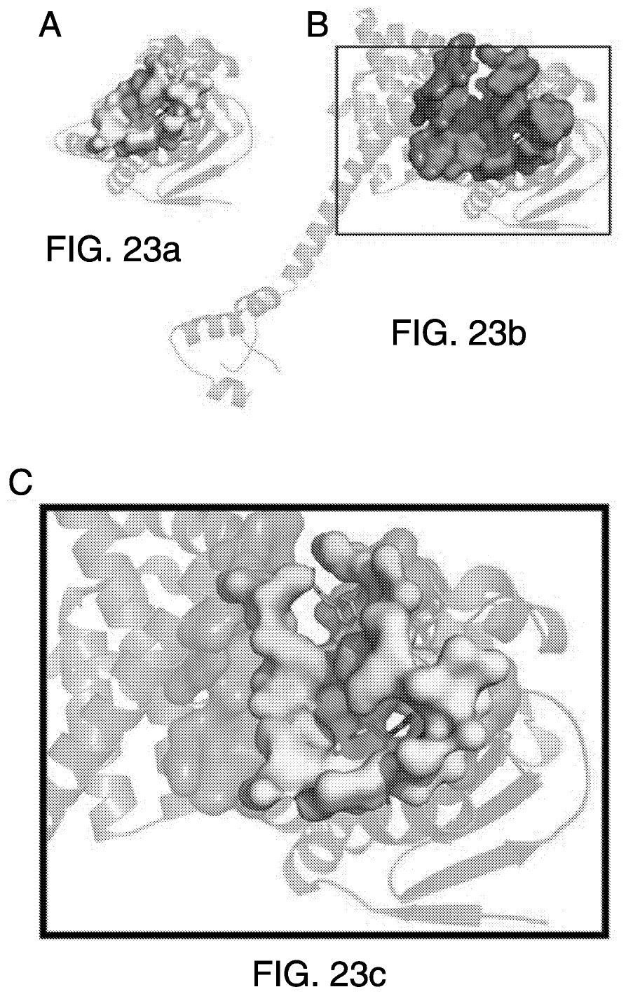

These combined experiments indicate that KBU2046 binds to HSP90β and CDC37 when both proteins are present, does not bind to either protein alone and together support the hypothesis that KBU2046 is binding in a cleft that is present when CDC37 and HSP90β interact. A comprehensive analysis of HSP90β and CDC37 experimental structural information, including X-ray crystallographic data (PDB IDs: 1uym, 3nmq, 3pry, 2cg9 and 1us7) and chemical cross-linker physical mapping analysis (Chavez, J. D. et al., Mol Cell Proteomics 12, 1451-67 (2013)), supports the notion that CDC37/HSP90β heterocomplex formation results in the formation of a new pocket, that is located at the interface of the two proteins. These modeling studies also predict that KBU2046 binds without any high energy steric interactions, and with a favorable energy score (FIGS. 5b-d and FIG. 23). In this computational arrangement, Arg167 from CDC37 protrudes into a large cleft, engages in a hydrogen bond with the carboxyl side chain of Glu33 from HSP90β, which promotes the formation of a new pocket, into which KBU2046 binds.

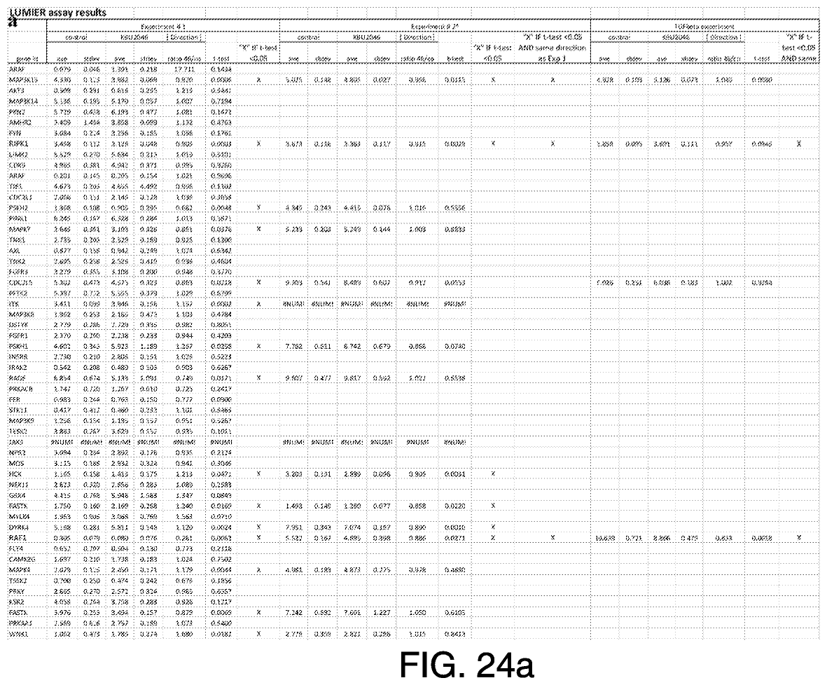

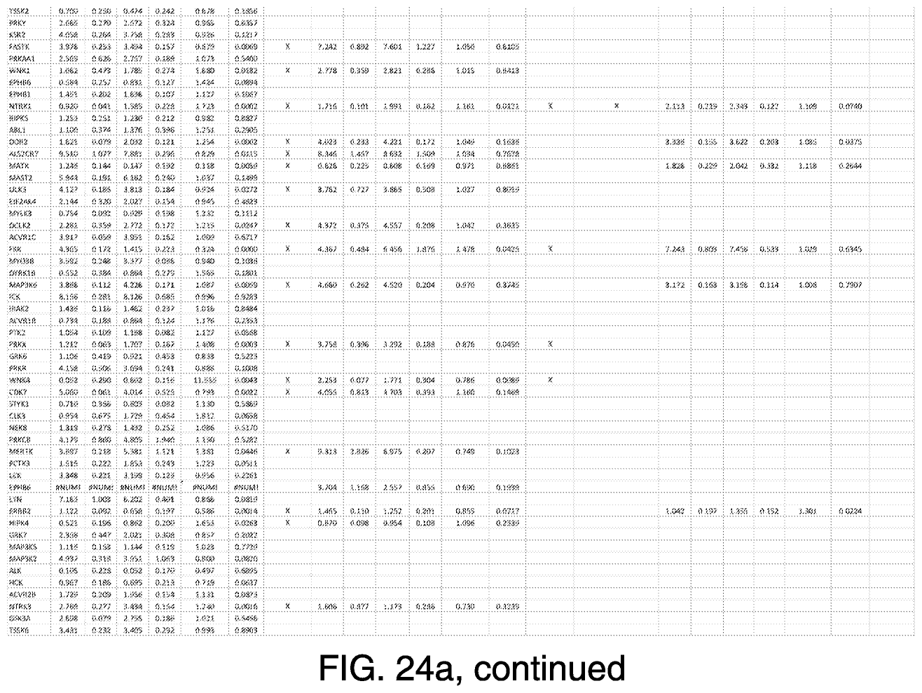

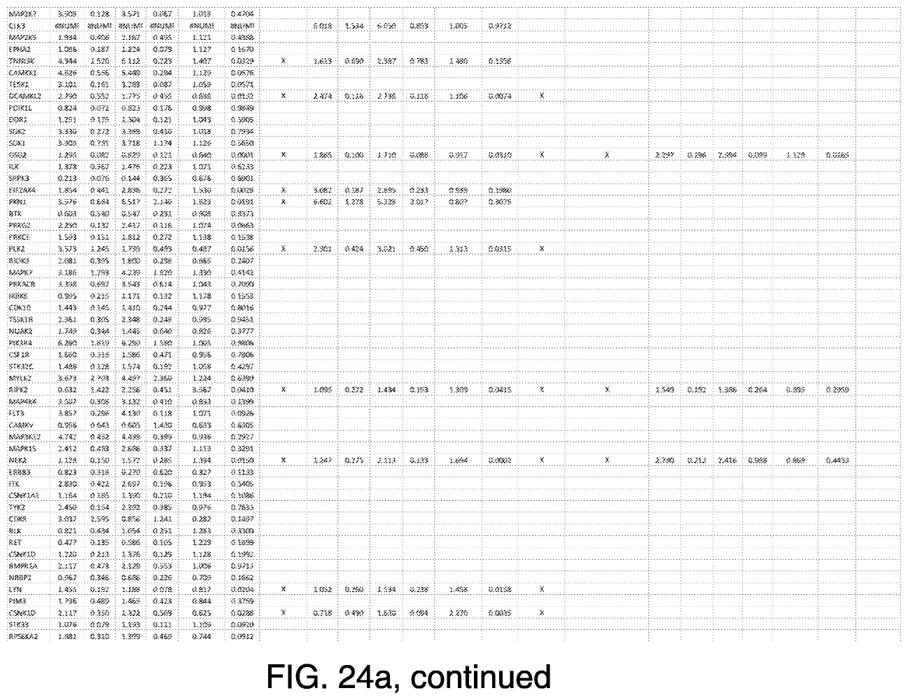

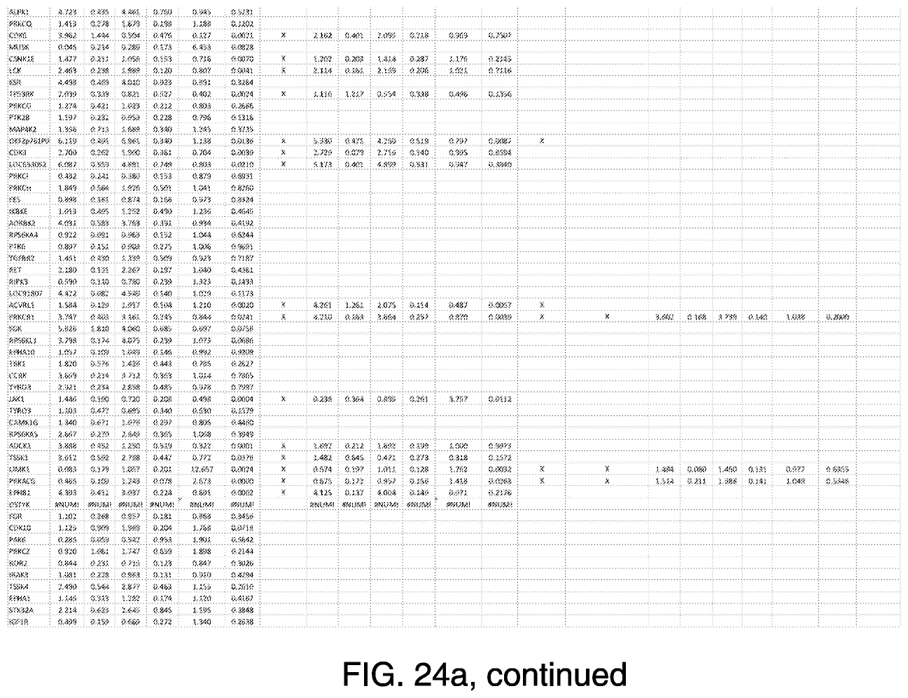

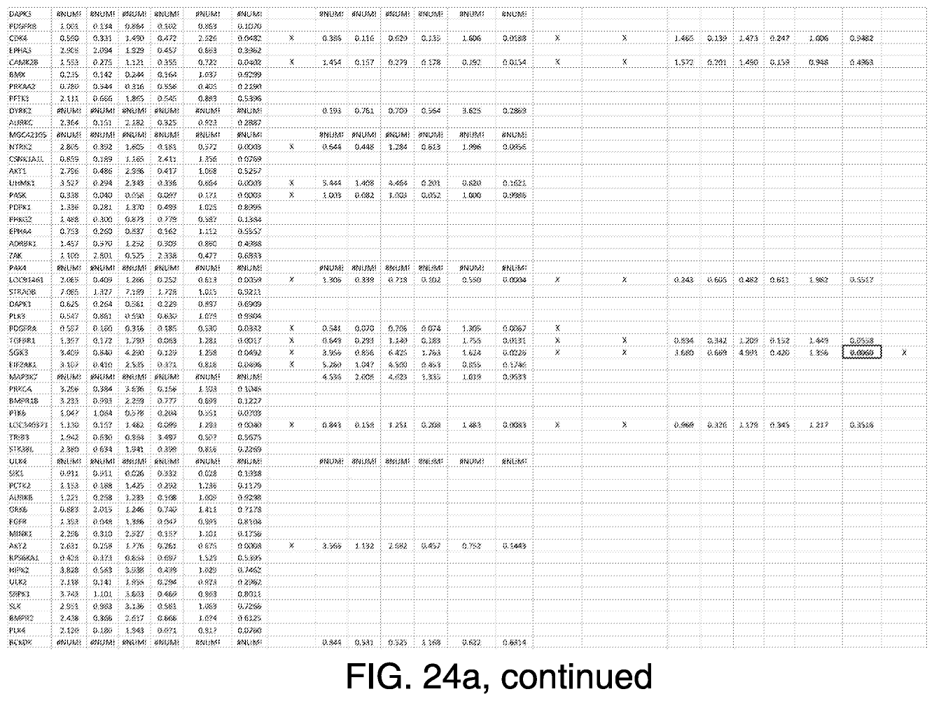

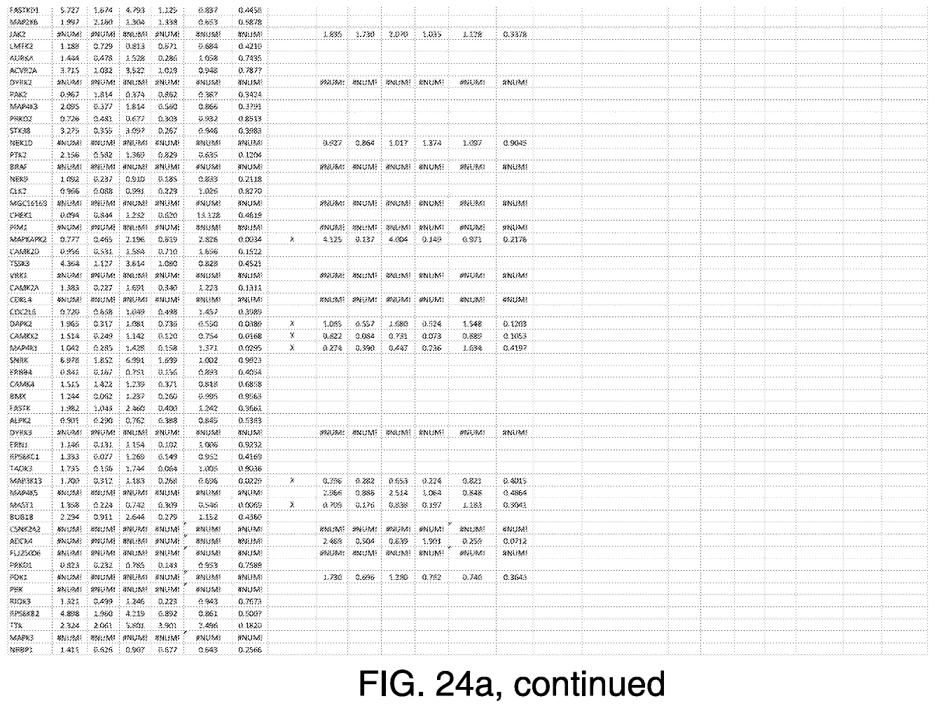

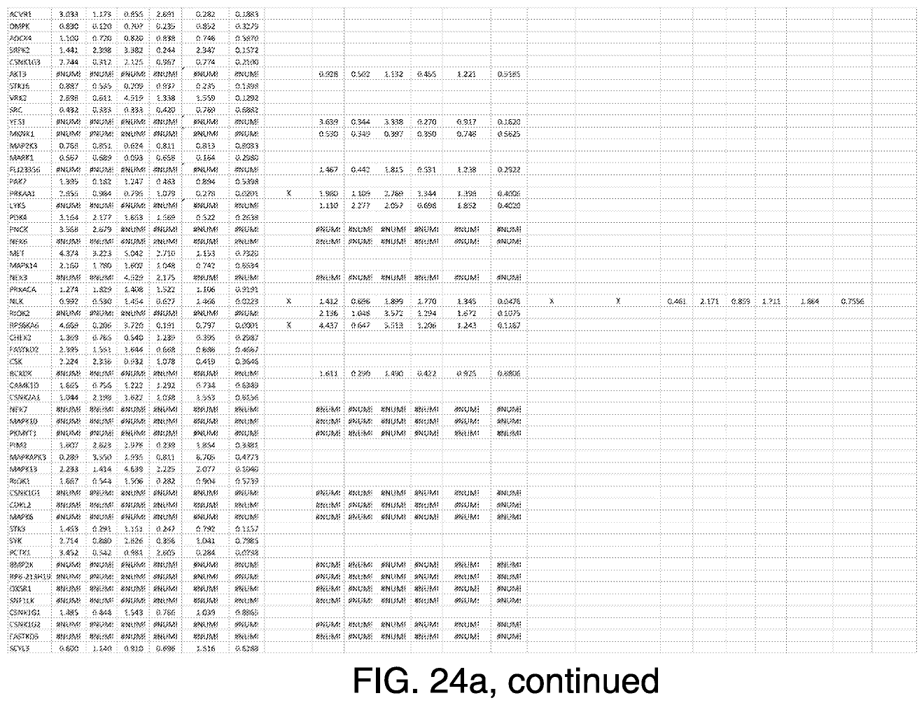

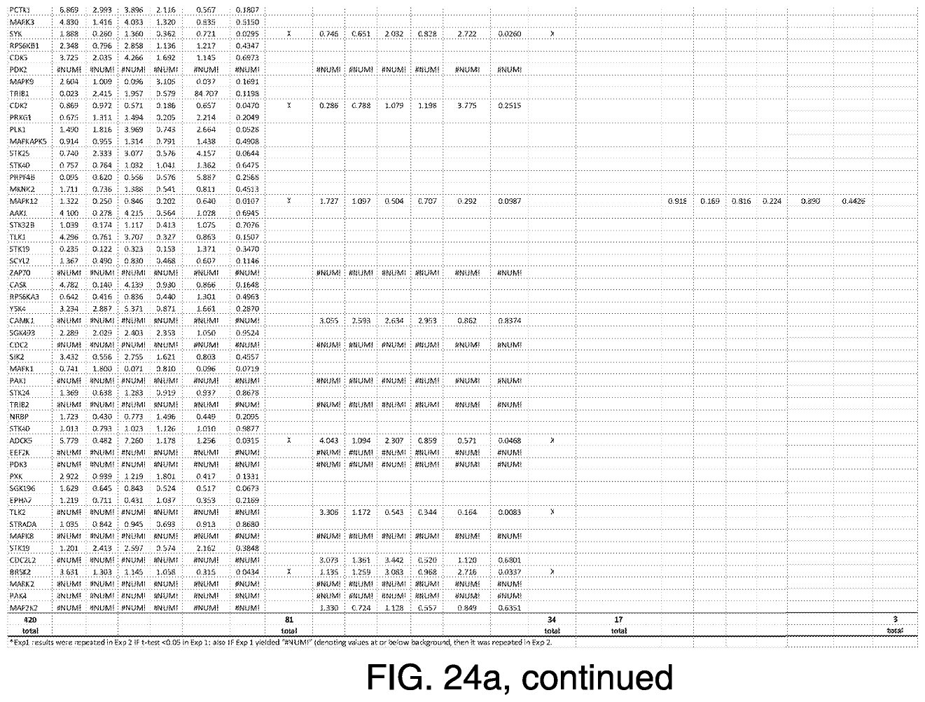

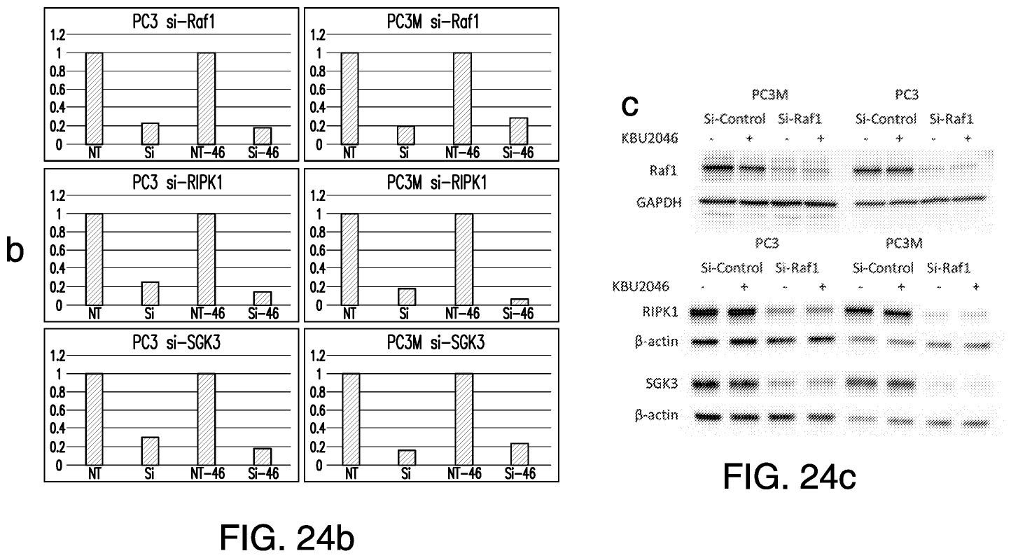

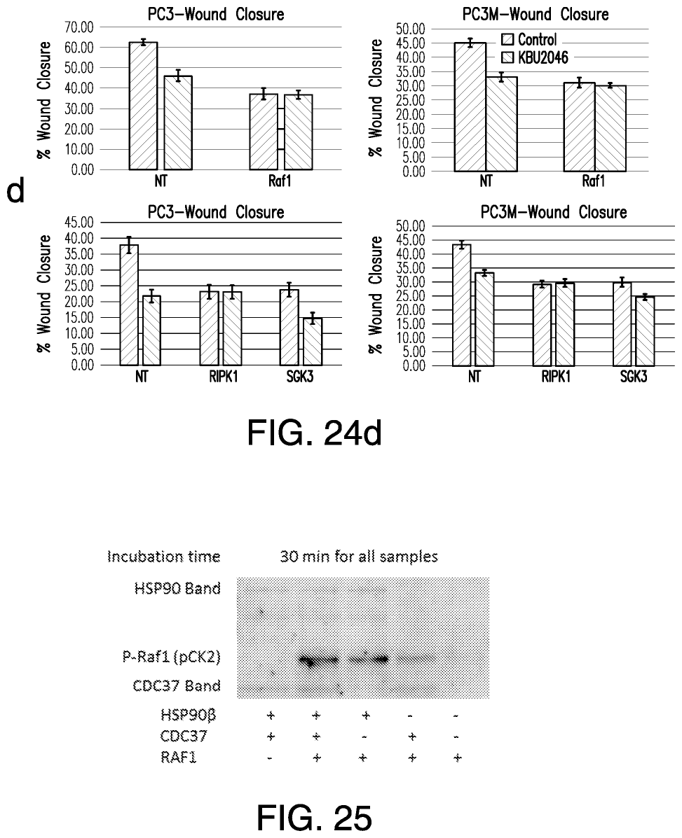

Together, these findings suggest that KBU2046 binds the CDC37/HSP90β heterocomplex. To examine whether this is associated with an altered signature of bound client kinase proteins, a modified LUMIER assay (Taipale, M. et al., Cell 150, 987-1001 (2012)) was performed to detect KBU2046-induced changes in client protein binding to CDC37/HSP90β heterocomplexes in intact cells. Of 420 kinase proteins screened, KBU2046 had highly selective effects, significantly changing the binding of 17 (4%): binding was increased in 10 and decreased in 7 (FIG. 6a and FIG. 24). These findings are in contrast to classical inhibitors of HSP90 function which have been shown, through this same assay, to affect the binding of the majority client kinase proteins (Taipale, M. et al., Cell 150, 987-1001 (2012)). Given that TGFβ increases cell motility and that KBU2046 efficacy remains in the face of TGFβ treatment (FIG. 18), the LUMIER assay was repeated in TGFβ treated cells, identifying 3 kinases whose binding to complexes was significantly affected by KBU2046: RAF1 (decreased binding), RIPKI (decreased) and SGK3 (increased) (FIG. 6b and FIG. 24a). The three proteins have been shown by others to regulate cell motility, and the knockdown of any one of them decreases motility was demonstrated (FIGS. 6c-d and FIGS. 24b-d). However, knockdown of RAF1 or of RIPK1 (i.e., the two kinases whose binding to the heterocomplex was decreased by KBU2046) mitigated KBU2046 efficacy, while KBU2046 still retained efficacy in the face of SGK3 knockdown.

DARTS assay findings (FIG. 5a) supported the notion of a direct interaction. However, the LUMIER-based approach used intact cells treated for three days and was unable to determine whether KBU2046 was directly interacting with heterocomplexes. Additional studies were therefore undertaken. Studies focused on RAF1. RAF1 is known to regulate cell motility and metastasis in several cancer types (Maurer, G. et al., Oncogene 30, 3477-88 (2011)) while KBU2046's effect upon RIPK1-complex binding was minor and not further enhanced by TGFβ. KBU2046 did not alter RAF1 protein expression levels in cells, FIG. 6c. This is significant in that HSP90 inhibitors broadly inhibit chaperone activity, thereby decreasing client protein expression. Next, an in vitro kinase assay was constructed of purified recombinant RAF1, HSP90β and CDC37, and demonstrated that KBU2046 decreased phosphorylation of RAF1's Ser338 activation motif (FIG. 6e). In the absence of CDC37/HSP90β heterocomplex, RAF1 activity was much lower, indicating that this effect is heterocomplex dependent (FIG. 25).

As KBU2046 does not directly inhibit protein kinase activity, it was hypothesized that its ability to decrease HSP90β phosphorylation resulted from changes in the signature of bound client kinases to the heterocomplex. This was examined by considering that in intact cells KBU2046 increased SGK3 binding to the heterocomplex (FIG. 6a), an effect that was anticipated may in turn phosphorylate HSP90β. Utilizing the in vitro kinase assay, it was demonstrated that that SGK3 increased phosphorylation of HSP90β, and that phosphorylation was further increased in the presence of KBU2046 (FIG. 6f). Recognizing the complexity of the system and the dynamic nature of the client-chaperone complex it was suspected that KBU2046 mediated inhibition of HSP90β phosphorylation was not an isolated event (i.e. not mediated by a single kinase operating in isolation). This was explored in the in vitro system by investigating the interplay of multiple kinases. In intact cells KBU2046 increases MAP3K6 binding to complexes (FIG. 6a), but MAP3K6 is not predicted to phosphorylate the Ser²²⁶ motif. Further it was demonstrated that when MAP3K6 is added to the in vitro kinase assay system, SGK3-mediated phosphorylation of HSP90β is inhibited, and also decreases in the presence of KBU2046 (FIG. 6g), emulating what is seen in intact cells.

Next, the results demonstrate that KBU2046 inhibits phosphorylation of RAF1's Ser338 activation motif in intact human prostate cancer cells (FIG. 7). Specifically, KBU2046 decreased levels of phospho-RAF1 in a time-dependent manner in both PC3 and PC3-M cells.

Discussion. The knowledge that increased cancer cell motility drives development of metastasis and that metastasis is responsible for the majority of cancer related mortality has pushed the research community to identify regulators of these processes. A wide array of pathways has been shown to affect these processes. However, the identification of specific regulatory processes has been elusive, which has served as a roadblock to therapeutic modulation (Steeg, P. S., Nat Med 12, 895-904 (2006); and Steeg, P. S., Nat Med 12, 895-904 (2006)).

Described herein is a molecular probe strategy to address this longstanding problem. That strategy used efficient synthesis routes to generate small molecules, which were then used as biological probes. The findings demonstrate that precision modulation of cell motility could be achieved by selectively changing the signature of client kinase proteins bound to the HSP90β/CDC37 heterocomplex. Further, it was demonstrated that enrichment for changes in bound client kinase proteins affect motility. It was further demonstrated that in the context of this altered binding signature that one of the affected client proteins, RAF1, plays an important role in mediating KBU2046 efficacy. These findings constitute an uncommon and specific regulatory mechanism for human cancer cell motility, and resultant downstream effects upon metastasis, end organ destruction and survival.

In parallel with the identification of this regulatory mechanism, these studies provide for a small molecule, KBU2046, that can both serve as a biological probe and as a systemically active therapeutic. Activity is demonstrated across different cancer types and across different clinically relevant systemic models. Further, a comprehensive characterization of pharmacology and toxicity support practical therapeutic application.

Coincident with affecting the bound signature of client proteins, post-translational changes to HSP90β were also inhibited. They include a decrease in its phosphorylation status, with several of the findings pointing to phosphorylation of Ser²²⁶ as being particularly important. It is recognized that there are a relatively high number of potential phosphorylation sites on HSP90β and that the antibody used to probe phosphorylation cannot be considered specific for the Ser²²⁶ motif. However, these investigations involving point mutations at that site and the phospo-proteomics evaluation of proteins bound to the antibody do provide supportive evidence to speculate that this site is of regulatory importance.

Of high importance, KBU2046 lacked the hallmarks of classical HSP90 inhibitors. Specifically, KBU2046 was not cytotoxic, lacked systemic toxicity, did not decrease expression of client proteins and it did not broadly alter kinase function. Further, KBU2046 bound to HSP90β and CDC37 when both proteins were present and able to form heterocomplexes, and would not bind to HSP90β as an isolated protein nor to CDC37 as an isolated protein. This is in contrast to classical HSP90 inhibitors which are characterized by their ability to bind HSP90 as an isolated protein.

Presented herein is a structural model in which KBU2046 binds within a cleft that is created through the binding of HSP90 and CDC37, and exists at the interface between these two proteins. This model contains several strengths including, use of information from physical mapping, biochemical analysis, crystallographic structure and an approach that integrated this information. However, it also has an important weakness in that its final construct was in silico. Several other models also describe the structure of HSP90/CDC37 interactions (Verba, K. A. et al., Science 352, 1542-7 (2016)); and Pearl, Biopolymers 105, 594-607 (2016)). Those models differ from each other, and from the model described herein. For example, the instant model integrated information from physical mapping (based on chemical cross linkers), from structural information reported in the literature, from the findings implicating KBU2046 interaction with HSP90/CDC37 heterocomplexes, and took into consideration the heterocomplex structure (i.e., in the absence of bound client proteins). In contrast, one recent model used cyroEM to evaluate HSP90/CDC37 heterocomplex structure in the context of its binding to the CDK4 client protein (Verba, K. A. et al., Science 352, 1542-7 (2016)). There are many other potential explanations. It likely that each model describes a particular state, and different states are possible. Future investigations will need to be performed in order to determine the effects of bound versus unbound client protein, different client proteins and binding of small molecules on HSP90/CDC37 heterocomplex structure.

From these findings, an integrated structural and functional model is proposed. KBU2046 binds to a cleft that is formed when HSP90β and CDC37 bind to form a heterocomplex. This in turn affects the ability of the hetercomplex to bind client kinase proteins is a precise manner, selectively affecting those which regulate cell motility. Of these, RAF1 is of particular importance. KBU2046 decreases RAF1 binding to the heterocomplex, resulting in decreased activation, and inhibition of cell motility. KBU2046's precision-type of effect on chaperone function differentiates it from classical HSP90 inhibitors which broadly disrupt client protein function, and underlie KBU2046's lack of toxicity and selective modulation of cell motility.

Finally, an in vitro assay for measuring the effect of chaperone protein activity on client proteins is described. Specifically, how KBU2046 inhibits chaperone-mediated activation of RAF1 is demonstrated. The practical predictive value of this assay is supported by a comprehensive set of findings. They include demonstrating KBU2046-mediated inhibition of RAF1 phosphorylation in intact human prostate cancer cells, inhibition of metastasis and end organ destruction in several systemic models of human cancer, demonstrating that RAF1 drives cancer cell motility and is important for KBU2046 efficacy, and demonstrating that KBU2046 physically binds HSP90/CDC37 heterocomplexes.

Together, the findings of this study provide a rational platform to move investigations into humans. That platform includes mechanistic strategy and the physical tools with which to affect it. Also, this study provides proof of principle findings that through pharmacologic means it is possible to induce precision modulation of the signature of client protein binding to chaperone scaffold proteins, in turn resulting in highly selective functional effects at the cellular and systemic level. In parallel, this approach serves to inform us about novel pathways for regulating important biological processes. Finally, this approach which coupled efficient chemical synthesis routes with a well-designed phenotypic screening strategy has the potential to be broadly applied as a tool to interrogate other important biological processes.

### Example 3: Chemical Synthesis

Methods.

Procedure for Large-Scale Production of 4′-fluoroisoflavanone (KBU2046).

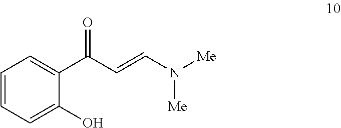 [SMILES: CN(C)/C=C/C(=O)c1ccccc1O]

3-(dimethylamino)-1-(2-hydroxyphenyl)prop-2-en-1-one. The starting materials 2′-hydroxyacetophenone (50 mmol, 6.02 mL) and N,N-dimethylformamide dimethyl acetal (50 mmol, 6.64 mL) were added to a 10-20 mL microwave vial. The vial was capped and heated in a Biotage Initiator microwave synthesizer at 150° C. and 11 bar for 10 minutes. The resulting dark orange liquid was allowed to cool to 23° C., at which time yellow-orange crystals crashed out of solution. The crystals were collected and washed with hexanes (50 mL), then dried and weighed to give 3-(dimethylamino)-1-(2-hydroxyphenyl)prop-2-en-1-one (9.09 g, 95%) as orange-yellow needles. Analytical data for 3-(dimethylamino)-1-(2-hydroxyphenyl)prop-2-en-1-one: ¹H nuclear magnetic resonance (NMR) (500 MHz, CDCl₃) δ 13.97 (s, 1H), 7.92 (d, J=12.1 Hz, 1H), 7.72 (dd, J=8.0, 1.6 Hz, 1H), 7.38 (ddd, J=8.5, 7.2, 1.6 Hz, 1H), 6.96 (dd, J=8.3, 1.2 Hz, 1H), 6.85 (ddd, J=8.3, 7.2, 1.2 Hz, 1H), 5.81 (d, J=12.1 Hz, 1H), 3.23 (s, 3H), 3.01 (s, 3H); ¹³C NMR (126 MHz, CDCl₃): δ 191.5, 163.0, 154.8, 134.0, 128.2, 120.3, 118.3, 118.0, 90.1, 45.5, 37.5; ultra-performance liquid chromatography/mass spectrometry (UPLCMS): Mass calculated for C₁₁H₁₃NO₂, [M+H]⁺, 192. Found 192.

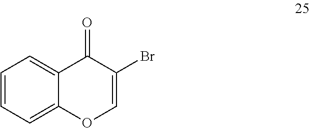 [SMILES: O=c1c(Br)coc2ccccc12]

3-bromochromone. 3-bromochromone was prepared by a procedure taken from Gammill (Gammill, R., Synthesis 1979, 901-903 (1979)). To a flame-dried 250 mL round bottom flask, was added 3-(dimethylamino)-1-(2-hydroxyphenyl)prop-2-en-1-one (36.6 mmol, 7.0 g), which was dissolved in CHCl₃ (70 mL). The reaction flask was cooled to 0° C. in an ice bath, then Br₂ (36.6 mmol, 1.87 mL) was added dropwise through an addition funnel. After all of the Br₂ was added, water (70 mL) was added slowly to the reaction and it was stirred at 23° C. for 10 minutes. The dark orange-yellow organic layer was then separated from the aqueous layer, which was back-extracted with 3×50 mL CHCl₃. The combined organic layers were then dried over Na₂SO₄ and concentrated to give a dark orange oil. This was purified by flash column chromatography (SiO₂, 10% EtOAc/hexanes) to afford 3-bromochromone (5.26 g, 64%) as an off-white solid. Analytical data for 3-bromochromone: ¹H NMR (500 MHz, CDCl₃) δ 8.31 (dd, J=8.0, 1.7 Hz, 1H), 8.27 (s, 1H), 7.75 (ddd, J=8.7, 7.1, 1.7 Hz, 1H), 7.57-7.44 (m, 2H); ¹³C NMR (126 MHz, CDCl₃): δ 172.3, 156.1, 153.8, 134.2, 126.5, 125.9, 123.2, 118.2, 110.7; UPLCMS: Mass calculated for C₉H₅BrO₂, [M+H]⁺, 226. Found 226.

Palladium tetrakis(triphenylphosphine) (Pd(PPh₃)₄). The catalyst for the Suzuki-Miyaura cross-coupling reaction to synthesize 4′-fluoroisoflavone was made using a procedure by Coulson (Coulson, D. R., Satek, L. C. & Grim, S. O. Tetrakis(Triphenylphosphine)Palladium(0). in Inorganic Syntheses: Reagents for Transition Metal Complex and Organometallic Syntheses, Vol. 28 (ed. Angelici, R. J.) (John Wiley & Sons, Inc., Hoboken, N.J., USA. 1990). To a flame-dried 100 mL Schlenk flask was added PdCl2 (5 mmol, 890 mg) and triphenylphosphine (25 mmol, 6.56 g). The solids were dissolved in DMSO (60 mL), then the mixture was purged with N2 and heated to 145° C., at which time it turned a bright yellow-orange color. The reaction was removed from heat and allowed to stir at room temperature for 15 minutes, then hydrazine hydrate (20 mmol, 0.972 mL) was added via syringe, with a vent needle in place to account for the formation of N₂ gas. After the hydrazine hydrate had been added, the reaction was cooled to 23° C., during which time a yellow solid crashed out of solution. The solid was washed under Schlenk filtration conditions with 2×50 mL EtOH, then 2×50 mL ether to yield Pd(PPh₃)₄ (5.31 g, 94%) as a canary yellow solid that was stored under N₂ in the glovebox.

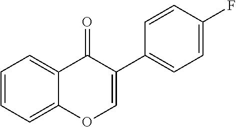 [SMILES: O=c1c(-c2ccc(F)cc2)coc2ccccc12]

4′-fluorisoflavone. 4′-fluoroisoflavone was prepared on large scale according to a procedure from Suzuki and Miyaura (Hoshino et al., Bull. Chem. Soc. Jpn. 61, 3008-3010 (1988)). To a flame-dried 500 mL round bottom flask was added 3-bromochromone (50 mmol, 11.25 g), 4-fluorophenylboronic acid (55 mmol, 7.69 g) and Na₂CO₃ (100 mmol, 10.6 g). The solids were dissolved in a mixture of benzene (100 mL) and water (50 mL), and the system was purged with N₂ for 10-15 minutes. The Pd(PPh₃)₄ catalyst (2.5 mmol, 2.89 g) was then added, at which time the reaction turned a bright orange. The flask was equipped with a reflux condenser and the reaction was heated to reflux (80° C.) overnight. After approximately 16 h, the reaction was cooled to 23° C. and was diluted with EtOAc (250 mL), then the crude material was passed through a plug of silica with EtOAc as the eluent. The organic material was dried over Na₂SO₄ and concentrated to give a dark brown solid that was adsorbed onto silica gel using DCM. Material purified by flash column chromatography (SiO₂, 20% EtOAc/hexanes) to afford 4′-fluoroisoflavone (8.14 g, 67% yield) as a yellow-orange solid that showed minor impurities by ¹H NMR spectroscopy. Slightly impure material was taken onto the next step of the synthesis without further purification. ¹H NMR (500 MHz, CDCl₃) δ 8.35 (dd, J=8.0, 1.6 Hz, 1H), 8.05 (s, 1H), 7.73 (ddd, J=8.7, 7.1, 1.7 Hz, 1H), 7.61-7.54 (m, 2H), 7.53 (dd, J=8.4, 1.1 Hz, 1H), 7.48 (ddd, J=8.2, 7.0, 1.1 Hz, 1H), 7.17 (ap t, J=8.7 Hz, 2H); ¹³C NMR (126 MHz, CDCl₃): δ 176.2, 163.8, 161.8, 156.2, 152.9, 133.8, 130.7, 127.8, 126.4, 125.4, 124.5, 118.1, 115.5; UPLCMS: Mass calculated for C₁₅H₉FO₂, [M+H]⁺, 241. Found 241.

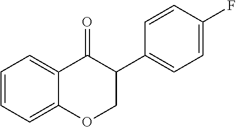 [SMILES: O=C1c2ccccc2OCC1c1ccc(F)cc1]

4′-fluoroisoflavanone (KBU2046). The reaction conditions to synthesize 4′-fluoroisoflavanone on large scale were adapted from a procedure reported by Wähälä (Salakka et al., Beilstein J Org Chem 2, 16 (2006)). To a flame-dried 500 mL round bottom flask was added 4′-fluoroisoflavone (25 mmol, 6.01 g), and the solid was dissolved in dry THF (100 mL). The solution was cooled to −78° C. (dry ice/acetone bath), monitored by a thermocouple. Once the solution had cooled to the desired temperature, L-selectride (55 mmol, 55 mL, 1 M solution in THF) was added dropwise over a period of 30-45 minutes. The reaction was then allowed to stir at −78° C. for 2 h, after which time it was quenched with MeOH (55 mL) at −78° C. The mixture was then poured into 300 mL of water, and the aqueous layer was adjusted to pH 7 with 2 M HCl. The aqueous layer was extracted 2×200 mL with EtOAc, then the combined organic layers were dried over Na₂SO₄ and concentrated to give a dark brown oily solid. This was purified by flash column chromatography (SiO₂, 1:1 hexanes:DCM) to give 4.5 g of crude material that was recrystallized in hexanes to afford 4′-fluoroisoflavanone (3.4 g, 56%) as a fluffy white solid. It was checked for purity by both ¹H NMR and HPLC analysis, with material that was >98% pure taken onto animal studies. Analytical data for isoflavanone 4′-fluoroisoflavanone: ¹H NMR (500 MHz, CDCl₃) δ 7.98 (dd, J=7.9, 1.7 Hz, 1H), 7.55 (ddd, J=8.6, 7.1, 1.7 Hz, 1H), 7.33-7.24 (m, 1H), 7.14-6.99 (m, 4H), 4.77-4.54 (m, 2H), 4.02 (dd, J=9.0, 5.3 Hz, 1H); ¹³C NMR (126 MHz, CDCl₃) δ 192.0, 163.3, 161.5, 136.2, 130.7, 130.2, 127.8, 121.8, 120.9, 117.9, 115.8, 71.4, 51.5; UPLCMS: Mass calculated for C₁₅H₁₁FO₂, [M+H]⁺, 243. Found 243.

Synthesis of additional compounds. A series of related analog compounds was synthesized in addition to the parent 4′-fluoroisoflavanone (KBU2046). These compounds were prepared in the same general manner of KBU2046 and the structures of each such compound are depicted in the Figures. The structure and purity of the additional analogs were confirmed by NMR spectroscopy (¹H and 13C) as well as by UPLCMS (minimal ion fragmentation). The compounds were isolated and stored in powdered form (in the absence of light) and were formulated into DSMO stock solutions just prior to use.

Cell Culture and Reagents. Prostate cancer (PC3, LNCaP, and DU145), breast cancer (MDA-MB-231 and MCF-7), colon cancer (HCT110 and HT29), and lung cancer cells (H226 and A549) were obtained from American Type Culture Collection. The origin, characteristics, for PC3-M, as well as for human papilloma virus (HPV) transformed primary 1532NPTX (normal), 1532CPTX (cancer), 1542NPTX (normal), and 1542CP3TX (cancer) cell lines, have previously been described (Liu, Y. Q. et al., Prostate Cancer Prostatic Dis 4, 81-91 (2001)). The origin of the stable polyclonal HEK293T cell lines expressing Renilla-HSP90β were previously described (Taipale, M. et al., Cell 150, 987-1001 (2012)). LM2-4H2N human breast cancer metastatic variant cells were derived from MDA-MB-231 cells as described (Francia, G. et al., Clin Cancer Res 15, 6358-66 (2009)), and the tdTomato-Luc2-expressing cell line was established by transduction of these cells with a lentiviral vector encoding fluorescent (tdTomato) and bioluminescent (Luc2) genes as described (Liu, H. et al., Proc Natl Acad Sci USA 107, 18115-20 (2010)). The cells were cultured as described (Liu, Y. Q. et al., Prostate Cancer Prostatic Dis 4, 81-91 (2001)); (Francia, G. et al., Clin Cancer Res 15, 6358-66 (2009)) were maintained at 37° C. in a humidified atmosphere of 5% carbon dioxide with biweekly media changes, were drawn from stored stock cells and replenished on a standardized periodic basis and were routinely monitored for Mycoplasma (PlasmoTest™, InvivoGen, San Diego, Calif.), at least every 3 months. Cells were authenticated by the following: they were acquired from the originator of that line, grown under quarantine conditions, expanded and stored as primary stocks and not used until following conditions were met: Mycoplasma negative; through morphologic examination; growth characteristics; hormone responsiveness or lack thereof, when applicable; replenished from primary stocks at least every 3 months; working with a single primary stock cell line at a time with hood sterilization in between.

Phospho-HSP27 (catalog #2401), phospho-p38 MAPK (#4631), p38 MAPK (#9212), Phospho-CK2 Substrate (#87385), CDC37 (#36185), HSP90β (#50875), GST (#26225), phospho-c-RAF (ser338) (#94275), SGK3 (#85735), and GAPDH (#2118) antibodies were purchased from Cell Signaling Technology. MAP3K6 (#SAB1300114) antibody, estradiol and 4′,5,7-trihydroxyisoflavone, genistein, were purchased from Sigma-Aldrich. The following recombinant proteins were purchased: Raf-1 (EMD Millipore; #17-360), MAP3K6 (Abnova; #P5592) and SGK3 (Thermo Scientific; #PV3859).

Cell Invasion Assays. Boyden chamber cell invasion assays were performed as previously described⁹, using either denatured collagen (BD Biosciences) or type IV human collagen (BD Biosciences), with the experiments repeated, each in N=4 replicates. In some experiments, as indicated, cells were transfected with an expression plasmid, using Lipofectamine 2000™ (Invitrogen), or with siRNA, using DharmaFect Duo (Thermo Scientific) and co-transfected with β-Galactosidase (Plasmid pCMV⋅SPORT-βgal; Life Technology).

Cell Growth Inhibition Assays. Three day MTT cell growth inhibition assays were performed as described (Liu, Y. et al., Oncogene 21, 8272-81 (2002)). Assays were in replicates of N=3, and were repeated.

Cell Migration Assays. Single cell motility assays were conducted by adding 10⁴ cells to 35 mm tissue culture dishes (BD Falcon) coated with collagen I (BD Biosciences), incubating at 37° C. in 5% CO₂, performing time-lapse imaging using a Biostation (Nikon Instruments), tracking the path of N≥35 cells using ImageJ software and the Manual Tracker plug-in, and using Chemotaxis and Migration Tool plug-in for data analysis.

Scratch Wound Assays. Cells were transfected with the indicated si-RNA constructs per manufacture protocol (GE-Dharmacon), cultured 48 hrs with 10 μM KBU2046 or vehicle, and scratch wound assay performed as described (Xiao, X. et al., Oncotarget 6, 3225-39 (2015)). The experiments were conducted in N=4 replicates, and repeated.

Constructs, Transfection, and Luciferase Assays. Constructs were purchased or gifts: constitutive active MEK4EE (MAP2K4-EE; residues 37-399; Addgene, plasmid 14813), pRL-TK-Renilla luciferase (Promega), pCMV-β-galactosidase (Agilent Technologies), and pcDNA-GFP (Invitrogen), HSP90β was from Pawel Bieganowski (Mossakowski Medical Research Center PAS, Poland) (Zurawska, A. et al., Biochim Biophys Acta 1803, 575-83 (2010)), estrogen responsive promoter-luciferase reporter construct, pERE-Luc, was from Craig Jordan (Georgetown University) (Catherino, W. H. et al., Cancer Lett 92, 39-47 (1995)), human CDC37 in pET15b plasmid was from Avrom Caplan (City College of New York) (Rao, J. et al., J Biol Chem 276, 5814-20 (2001)). siRNA used Dharmacon ON-Targetplus SMARTpool™ siRNA directed against HSP90β (cat #L-005187-00-0010) non-targeting siRNA (cat #D-001810-10-05), used TransIT-LT1 Transfection Reagent (Mirus Bio LLC), or with Dharmafect Duo (Thermo Scientific, Lafayette, Colo.) for co-delivery of plasmid. Luciferase assays were performed as described (Breen, M. J. et al., PLoS One 8, e72407 (2013)).

Animal Models of Metastasis and of Systemic Effects. The animals were housed in barrier (for immunocompromised mice) or conventional facilities, with a 12-hour light/dark cycle and given soy-free food and water ad libitum. Animal study sample size determination: Sample sizes were determined using the samples size estimation formula for differences in means with power set 80%, 2-sided alpha=0.05, and a pre-specified effect size of 30%.

Prostate cancer: orthotopic implantation. Orthotopic implantation of human GFP-PC3-M PCa cells into 6-8 week male Balb/c athymic mice (Charles River Laboratories) and quantification of distant metastasis was performed as described (Pavese, J. et al., J Vis Exp, e50873 (2013)). Treatment with KBU2046, incorporated into Harlan Teklad 20165® chow, began one week prior to implantation. Animals were excluded from the analysis if they died and/or met the criteria for euthanasia in the 7 day post-operative period. Experimental groups were randomly assigned to cages prior to the initiation of the study. Metastasis were scored in a blinded fashion. Specifically, animals were assigned an ID number, resultant histologic slides contained a separate pathological ID number, slides were scored in a random fashion, after which the two numbers were linked up.

Breast cancer: orthotopic implantation. Orthotopic implantation of 2×10⁶ dTomato-LM2-4H2N cells in matrigel:PBS human breast cancer cells into 5-6 week old female SCID-Beige mice (Taconic), followed by resection of resultant primary tumor, provides a model wherein survival is dictated by metastatic burden, and was performed as described (du Manoir, J. M. et al., Clin Cancer Res 12, 904-16 (2006)). KBU2046 treatment by oral gavage 5 days/wk began 4 wks after resection, with weekly IVIS imaging. Animals were excluded from the analysis if they died and/or met the criteria for euthanasia in the 7 day post-operative period. Experimental groups were randomly assigned based upon size of primary tumor before treatment, in and manner that ensured equal distribution of tumor sizes across treatment groups.

Prostate cancer: intracardiac (IC) injection. IC injection of 4.0×10\hat{ }5 PC3-Luc cells into male 6-8 week old athymic mice with IVIS imaging was performed as described (Chu, K. et al., Mol Cancer Res 6, 1259-67 (2008)), and was done so under ultrasound guidance (FIG. 15). Mice were treated pre- and/or post-IC injection, as indicated. The pretreatment cohort emulates a metastasis naïve model wherein cell motility is required in order for cells to invade into a distant organ, in this case the jaw bone, for which this model is widely used. If KBU2046 were inhibiting cell motility, then in this model it would act to inhibit metastasis formation. In contrast, with the post implantation cohort, cells have already completed invasion into the jaw bone, and distant metastasis have been established. If KBU2046 were inhibiting cell motility, then in this model, it should exhibit no efficacy. IVIS imaging was performed 30 minutes after IC injection to confirm systemic distribution of cells, and thereafter weekly for 4 weeks starting seven days post injection, allowing real time tracking of metastasis development and growth. Computed Tomograpgy (CT) (Inveon, Seimens) radiographic imaging was performed on some mice, as indicated, and resultant images analyzed in a blinded fashion using Inveon Research Workplace 4.2 visualization/analysis software and ImageJ. Animals were excluded from the analysis if 30 min post injection IVIS imaging revealed a focal signal in the chest indicating a failed injection where cells were not distributed into circulation. Experimental groups were randomly assigned to cages prior to the initiation of the study. CT images were analyzed in a blinded fashion to determine total bone loss using both the Invenon Research Workplace 4.2 visualization/analysis software and ImageJ. Specifically, to assess bone loss in an unbiased manor, an operator randomly loaded images into the system and blocked out identifying information prior to analysis by two separate blinded individuals.

Systemic effects. Off target effects were sought by performing histologic examination of tissue and by measuring organ function.

Histologic examination of tissue. Organs of athymic male mice stained with H&E, Trichrome or Giemsa were microscopically examined by a mouse pathologist in a blinded fashion, and toxicity scored using an established semi-quantitative histological scoring system (Knodell, R. G. et al., Hepatology 1, 431-5 (1981)) (FIG. 14). The following clinical chemistry parameters were measured in blood of CD1 male and female mice by Charles River Research Animal Diagnostic Services: cholesterol, triglycerides, alanine aminotransferase (ALT), aspartate aminotransferase (AST), total bilirubin, glucose, phosphorus, total protein, calcium, blood urea nitrogen (BUN), creatinine, albumin, Na, K, Cl, white blood cells (with differential), red blood cells, hemoglobin, and platelets.

Hematopoietic Stem Cell Colony Formation Assay. Fourteen-day colony formation assays were conducted as described (Bergan, R. et al., Blood 88, 731-41 (1996)), using human cord blood CD34+ stem cells (AllCells Inc.), using MethoCultExpress™ colony growth media (StemCell Technologies Inc.), performed in replicates of N=2.

KBU2046 Quantification and Pharmacokinetics. For determination of pharmacokinetic (PK) parameters, KBU2046 was administered by either intravenous injection or oral gavage to groups of N=3 CD1 (ICR) mice at 0 (i.e., vehicle), 25, or 100 mg/kg. Blood was collected into EDTA by terminal cardiac puncture before drug administration (i.e., baseline) and at 5, 10, 15, 30, 45, 60, 90, 120, 180, 240, 360, 480, 960, 1440 minutes after administration. In separate experiments, as described herein, Balb/c athymic mice were dosed with KBU2046 incorporated into chow for 35 days, after which blood was collected by terminal cardiac puncture. Resultant plasma (approximately 250 μl/mouse) was stored at −80° C.

Plasma KBU2046 concentrations were measured in duplicate by liquid chromatography-tandem mass spectrometry after sample preparation by solid-phase extraction. Specifically, 0.1 mL of a sample, 10 μL of 0.1 μg/mL internal standard solution (3-(2-chlorophenyl)-(4H-1-benzopyran-4-one, a chloride analog of KBU2046), 3800 μL of water, and 10 μl of 85% phosphoric acid were added, vortexed, and stored at 4° C. for 2 hours. After washing a 96-well Strata-X 33 μm Polymeric Reversed Phase 30 mg/well solid-phase extraction plate (Phenomenex) with methanol and water, sample was applied, washed with 20% methanol in water, eluted with 70% acetonitrile/30% methanol, dried at 50° C. under N₂, reconstituted with 100 μL of mobile phase, and 20 μl was analyzed on an API 3000 liquid chromatography-tandem mass spectrometry system (Applied Biosystems) with an Agilent 1100 series HPLC system (Agilent Technologies). Samples were eluted isocratically from a Synergi 4-μm MAX-RP 100 Å column (2.0×50 mm; Phenomenex) by a mobile phase consisting of 10 mM ammonium formate in water and methanol (30:70 [vol/vol]) at a flow rate of 0.20 mL/min. The tandem mass spectrometer was operated with its electrospray source in the positive ionization mode. The mass-to-charge ratios of the precursor-to-product ion reactions monitored were 243.5→125.1 for KBU2046 and 257.0→165.0 for the internal standard. The retention time of KBU2046 was approximately 2.7 minutes while that of the internal standard was approximately 2.3 minutes. The linear range for plasma KBU2046 standard curves was 0.1 to 25.0 ng/mL, with coefficients of variation of 10% or less throughout the entire concentration range. Fresh plasma standard curves were prepared in blank plasma and run on the day of analysis of plasma samples.

Plasma KBU2046 concentration versus time relationships after both intravenous and oral drug administration were modeled simultaneously using the SAAM II software system (SAAM Institute), implemented on a Windows™-based PC (see PK modeling schema). Plasma concentrations were modeled with a three-compartment PK model using a naïve pooled data approach (Kataria, B. K. et al., Anesthesiology 80, 104-22 (1994)). Oral drug absorption was characterized by a tanks-in-series delay element, to account for the non-instantaneous appearance of drug in the body. Simultaneous estimation of PK model parameters for both routes of administration permitted estimation of the bioavailability of the orally administered drug (F) (Avram, M. J. et al., Clin Pharmacol Ther 85, 71-7 (2009)). The SAAM II objective function used was the extended least-squares maximum likelihood function using data weighted with the inverse of the model-based variance of the data at the observation times (Barrett, P. H. et al., Metabolism 47, 484-92 (1998)). Model misspecification was sought by visual inspection of the measured and predicted marker concentrations versus time relationships (Barrett, P. H. et al., Metabolism 47, 484-92 (1998); and Cobelli, C. et al., Adv Exp Med Biol 445, 79-101 (1998)).

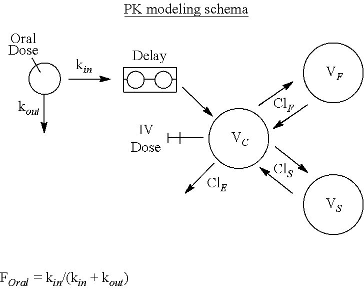

Quantitative Reverse Transcriptase Polymerase Chain Reaction. RNA was isolated and qRT/PCR was performed as described (Ding, Y. et al., J Biomol Tech 18, 321-30 (2007)), analyzed by the 2^{-ΔΔCt} method (Livak, K. J. et al., Methods 25, 402-8 (2001)), using primer/probes sets (ABI), HSP90α (Hs00743767_sH), HSP90β (Hs00427665_g1), trefoil factor 1 (TFF1; Hs00907239 ml), cathepsin D (CTSD; Hs00157205_m1), progesterone receptor (PGR; Hs01556702 ml) and GAPDH (Hs99999905_m1). Assays were repeated, each in replicates of N=2.

Biophysical and Biochemical Binding Assays. Fluorescent thermal shift (Krishna, S. N. et al., PLoS One 8, e81504 (2013)), isothermal titration calorimetry (Chavez, J. D. et al., Mol Cell Proteomics 12, 1451-67 (2013)), and biolayer interferometry assays (Makowska-Grzyska, M. et al., Biochemistry 51, 6148-63 (2012)), were performed using MEK4EE (37-399, S257E, T261E) cloned into pMCSG7 and KRX Competent E. coli cells (Promega Inc.) and full-length HSP90B and CDC37 vectors and Rosetta BL-21 and BL-21 DE-3 competent E. coli cells, respectively. Drug affinity responsive target stability (DARTS) assays were conducted as described (Lomenick, B. et al., Proc Natl Acad Sci USA 106, 21984-9 (2009)), using equimolar amounts of CDC37 and HSP90β, thermolysin digestion and silver stain visualization (ProteoSilver Silver stain kit, Sigma Aldrich).

In Vitro Kinase Assays. MEK4/MKK4/MAP2K4 in vitro kinase assay was performed as described (Krishna, S. N. et al., PLoS One 8, e81504 (2013)). HSP90β/CDC37 heterocomplex kinase assays with RAF1, SGK3 and MAP3K6 assays incubated indicated proteins in 20 mM MOPS, pH 7.2, 25 mM β-glycerol phosphate, 5 mM EGTA, 1 mM sodium orthovanadate, 1 mM dithiothreitol, 0.25 mM ATP and 37.5 mM MgCl2 at 30° C. for the indicated times.

Phospho-proteomic Analysis. The Kinoview™ and PhosphoScan™ assays were performed by Cell Signaling Technology Inc.

Protein Microarray Binding Assay. The ProtoArray® assay, using the Human Protein Microarray platform, was performed by Invitrogen.

High-Performance Molecular Modeling Platform. Modeling and docking used the APPLIED Pipeline (Analysis Pipeline for Protein Ligand Interactions and Experimental Determination) at the Argonne Leadership Computing Facility, Argonne National Laboratory, tuned for the 786,432 core BlueGene/Q Mira (Zhao, Y. et al., (Springer, 2007)), using a multi-stage pipeline that considers protein-protein/ligand interactions through evolutionary protein surface analysis (Binkowski, T. A. et al., J Mol Biol 332, 505-26 (2003); Binkowski, T. A. et al., BMC Struct Biol 8, 45 (2008); and Binkowski, T. A. et al., Protein Sci 14, 2972-81 (2005)), robust homology modeling (Leaver-Fay, A. et al., Methods Enzymol 487, 545-74 (2011)), massively parallel docking simulations using mixed strategies (Lang, P. T. et al., RNA 15, 1219-30 (2009); Graves, A. P. et al., J Mol Biol 377, 914-34 (2008); Morris, G. M. et al., J Comput Chem 30, 2785-91 (2009); Deng, Y. et al., J Chem Phys 128, 115103 (2008); and Wang, J. et al., Biophys J 91, 2798-814 (2006)), and advanced, physics-based rescoring methodologies.

LUMIER assay. The LUMIER assay was performed as described (Taipale, M. et al., Cell 150, 987-1001 (2012)).

Statistical Analysis. The results were analyzed by a statistician. Unless otherwise stated, statistical significance was evaluated with the two-sided Student's t-test using a threshold of P≤0.05. The experiments, unless otherwise stated, were conducted in replicates of at least N=2-6 (with specific N values are denoted for each experiment) and were repeated at a separate time, also in replicates of N=2-6. The relationship between dose and metastasis, and between drug concentration and effect on protein degradation, was evaluated by two-sided ANOVA. The survival of mice was evaluated by the log rank (Mantel-Cox) test.

### Example 4: The Synthetic Strategy

Beginning with 4′,5,7-trihydroxyisoflavone (genistein) as a chemical scaffold, a fragment-based chemical diversification synthesis approach was pursued, and coupled in an iterative fashion to biological assays of cell invasion and cell growth inhibition. Compounds that inhibited cell invasion but did not inhibit cell growth were selected for further modification and evaluation. The initial round of synthesis was designed to examine the removal of individual chemical fragments. In this manner, the importance of these functional groups in mediating efficacy (inhibition of cell invasion) could be determined. Subsequent rounds built upon refined structure activity relationship (SAR) knowledge, and sought to improve efficacy, while deselecting for toxicity (cell growth inhibition). Initial assays were performed with PC3 and PC3-M cells. However, as these studies yielded similar findings, subsequent screening assays utilized only PC3-M cells. In designing chemical synthetic routes, priority was given to efficacy and toxicity parameters. Additional design features were also included in the chemical synthetic routes, but they were incorporated if they did not compromise efficacy and toxicity parameters. These additional design features included removal of fragments that mediated genistein binding to the estrogen receptor (ER), as determined by ER-genistein 3D x-ray crystal structures (Protein Data Base IDs: 1X7R and 1X7J, for crystal structures of ERα and ERβ with bound genistein, respectively). These features also included removal of chemical fragments that are considered to increase susceptibility to rapid metabolism, for example by the cytochrome P450 (CYP) pathway. The final feature involved incorporation of chemical characteristics previously shown to be associated with effective drugs and which together generally impart more favorable pharmacologic properties, including those described by Lipinski et al. (Lipinski, C. A. et al., Adv Drug Deliv Rev 46, 3-26 (2001)).

FIG. 7 shows mean±SEM of three separate experiments, each run in replicates of N=3. Three day MTT cell growth inhibition assays were performed with PC3-M cells. Values are the mean±SEM of a single experiment, in replicates of N=4, repeated at least once (also N=4).

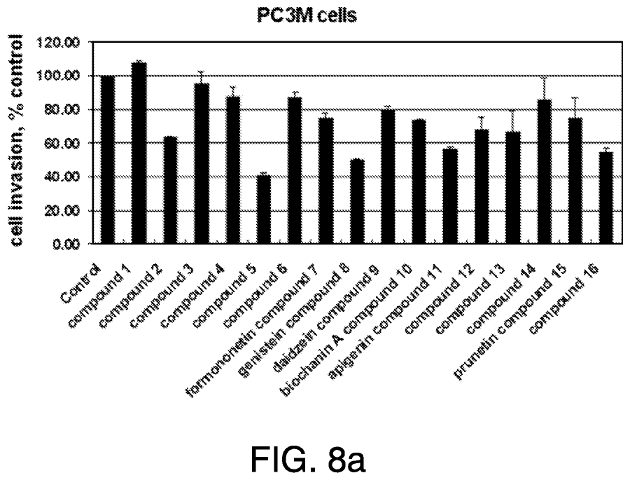

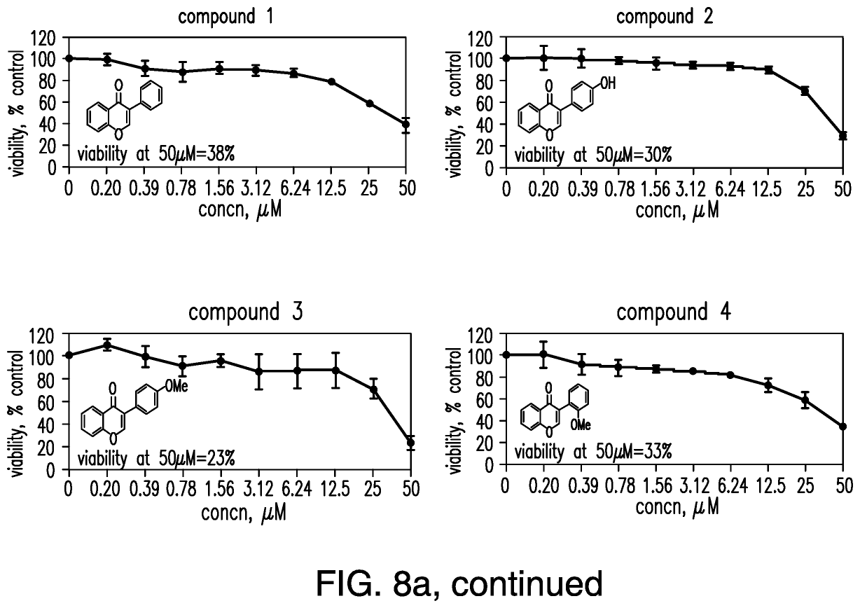

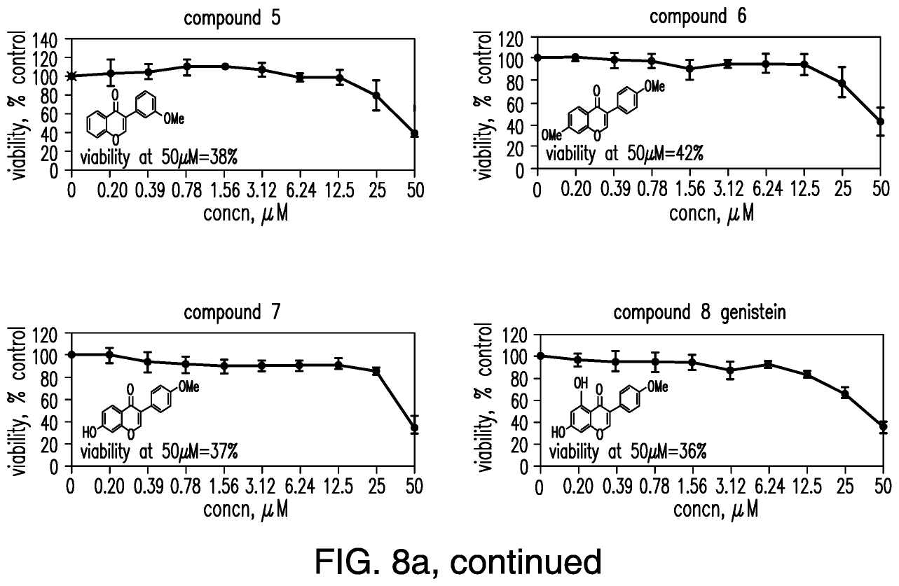

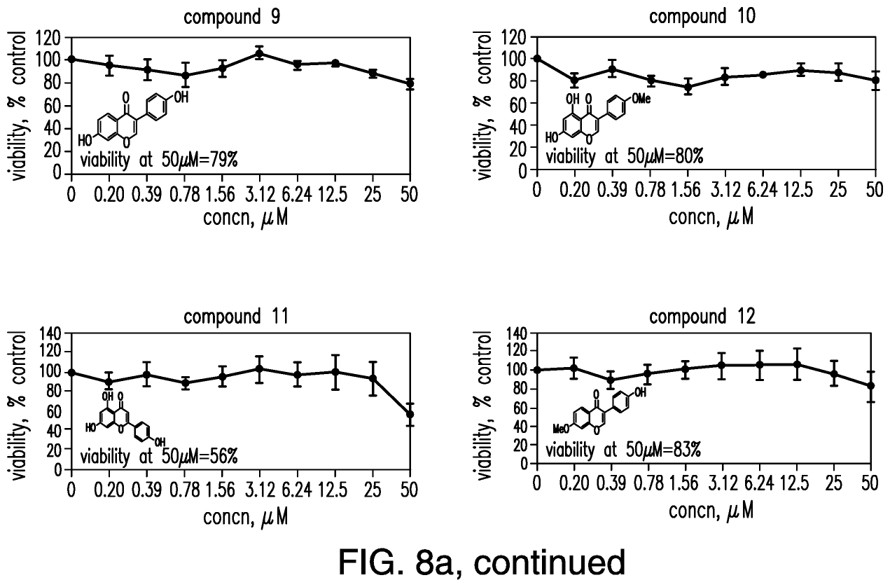

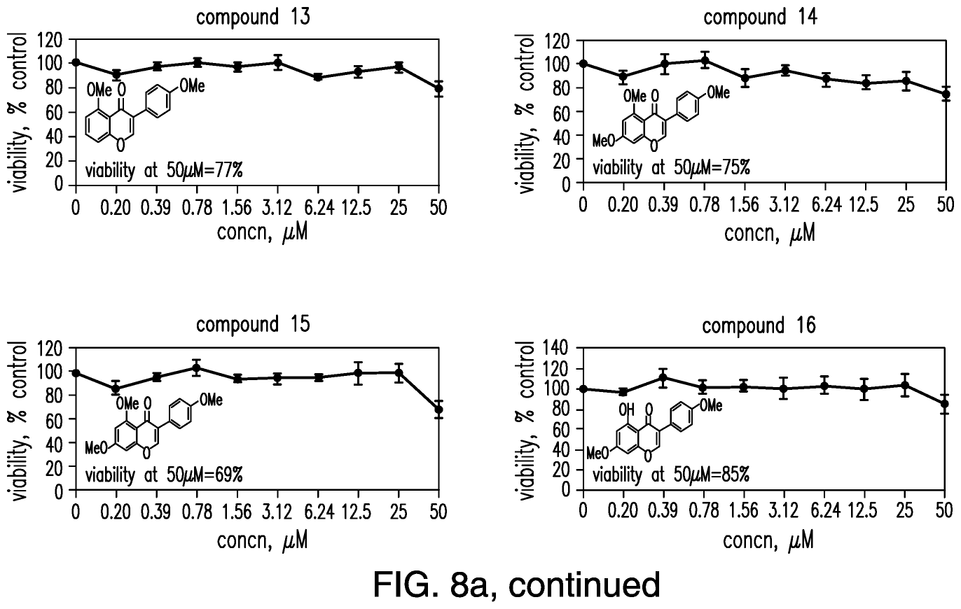

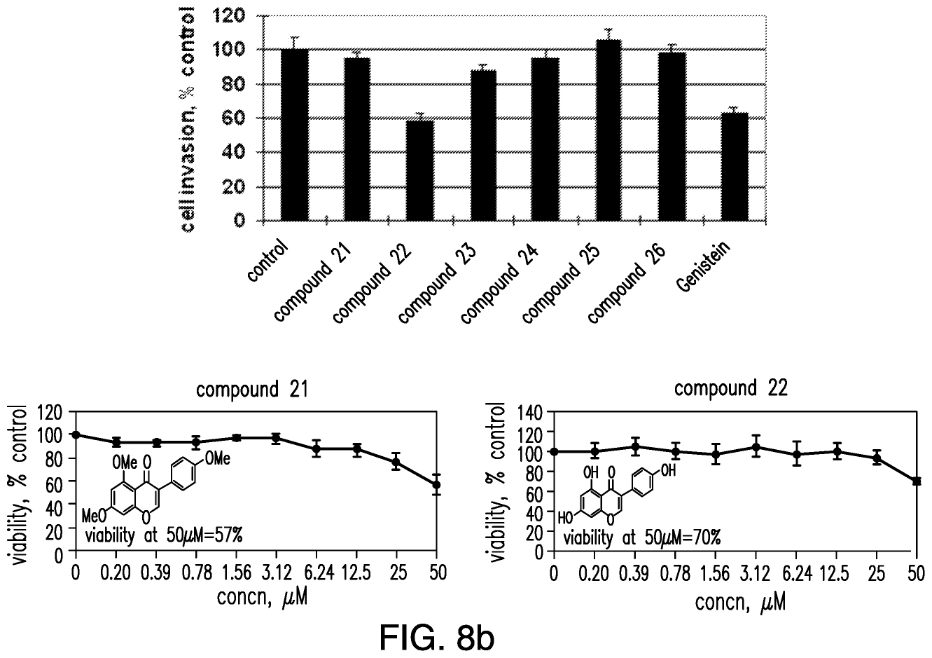

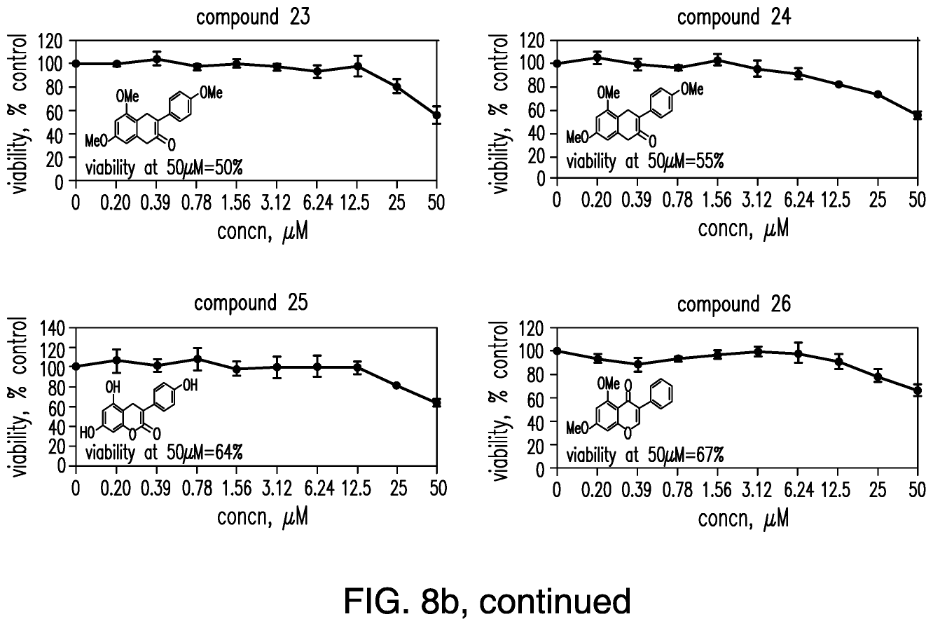

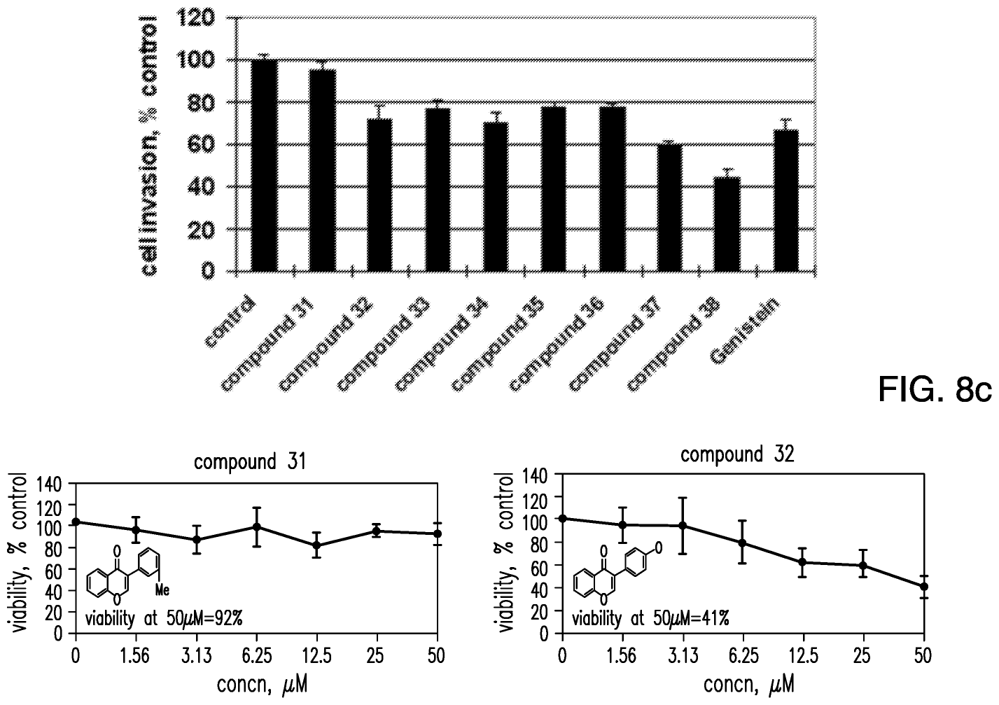

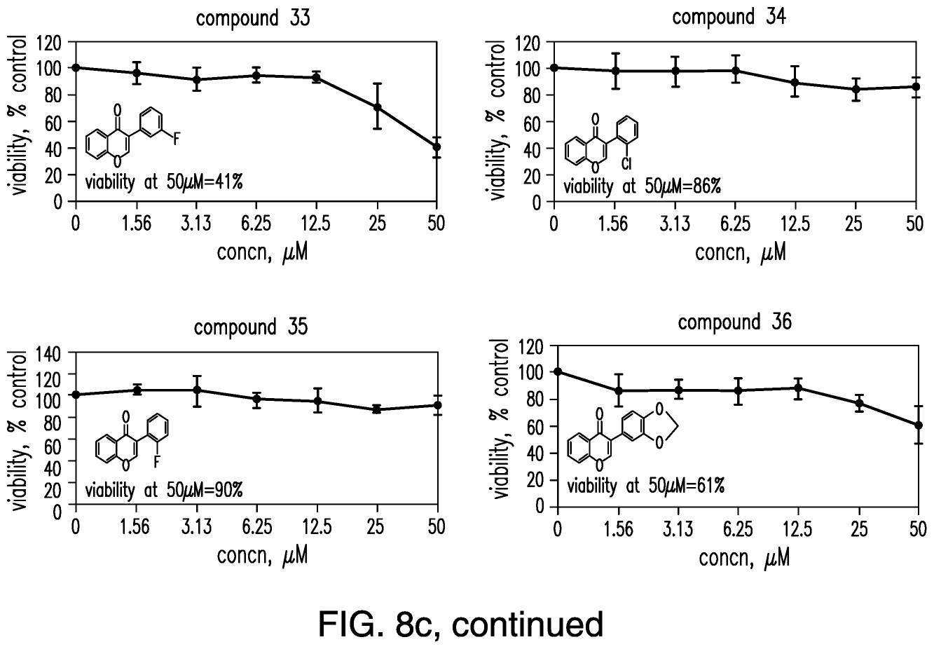

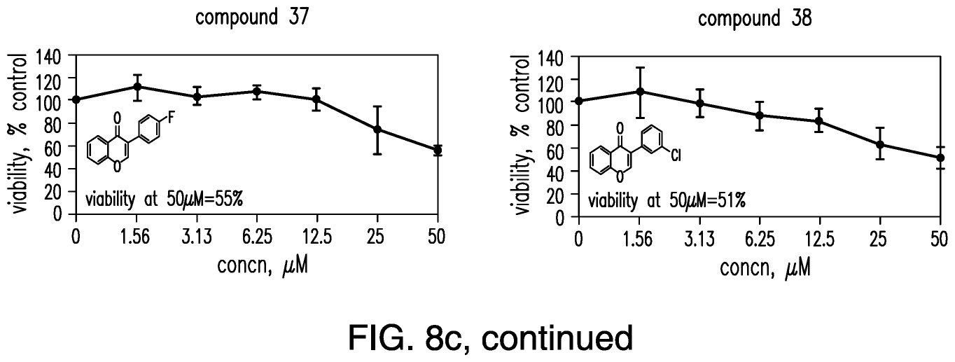

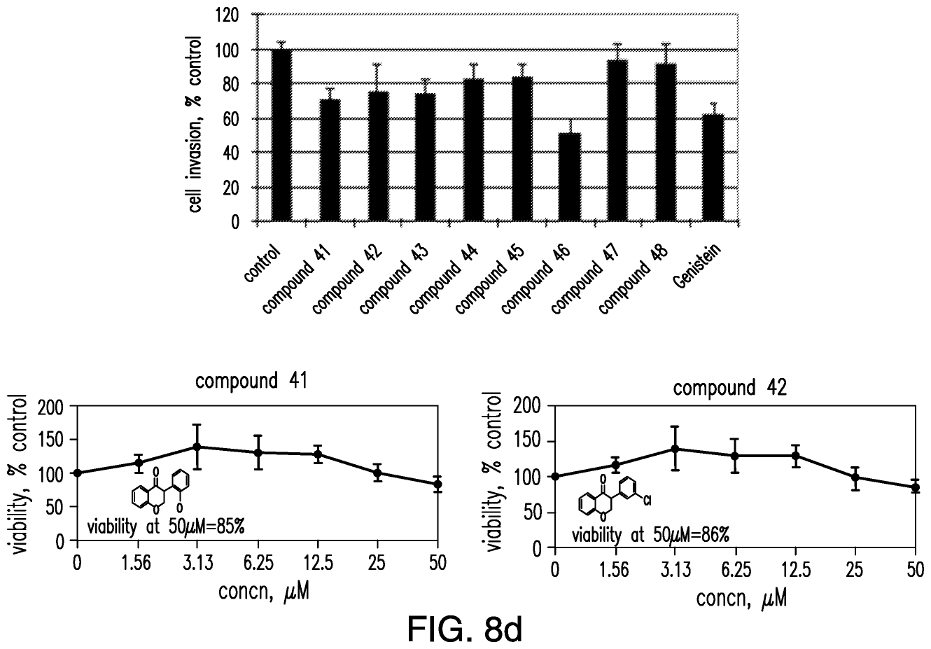

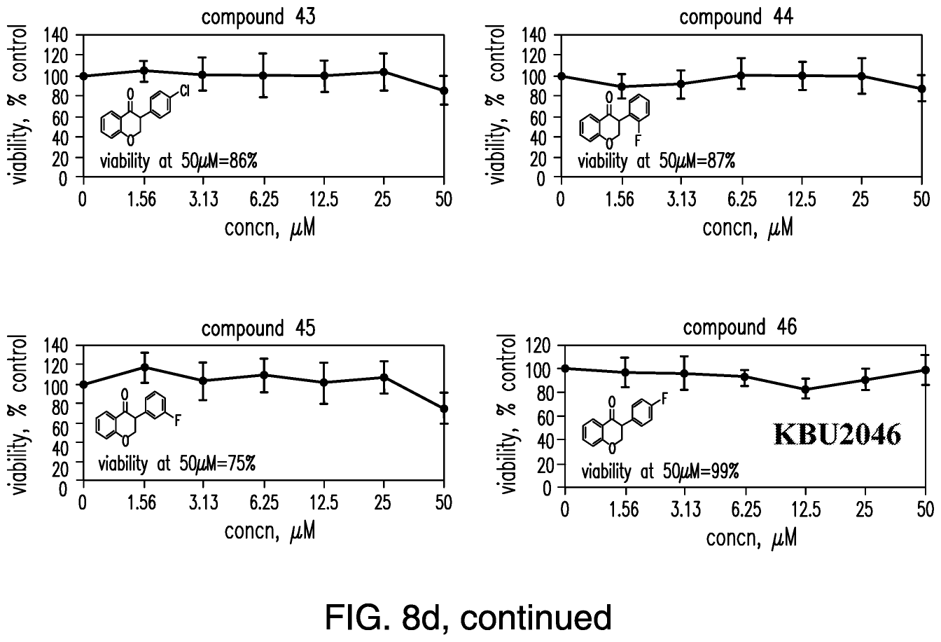

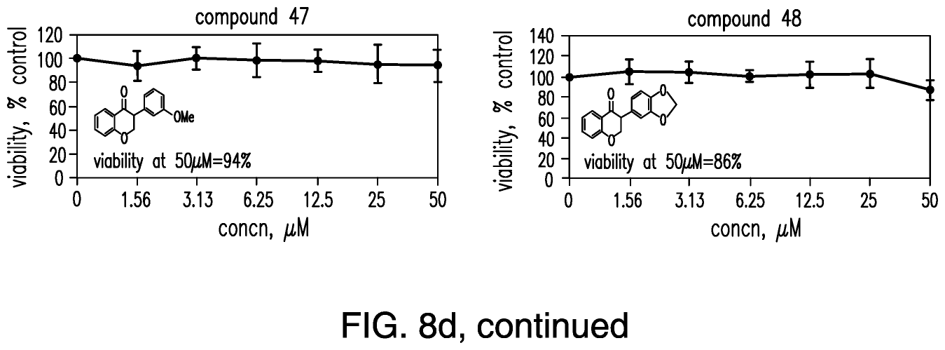

FIG. 8c shows synthetic round #3. Key findings include but are not limited to: substitution of the C4′-hydroxyl group with a halide is associated with maintenance of activity (compounds 37 and 38). A new chemical entity was identified with potent anti-invasive effects, but which still retains growth inhibitory effects (compound 38). As shown in FIG. 10, estrogen receptor positive MCF-7 cells were cultured under hormone free conditions transfected pERE-Luc or empty control vector, along with constitutive active β-gal, grown under estrogen-free conditions, and pre-treated for 24 hours with nanomolar concentrations of estradiol, or with micromolar concentrations of genistein or KBU2046, as indicated. Luciferase activity was measured, normalized to that of β-gal, and values expressed as the percent of untreated vector control cells.

As shown in FIG. 11, in order for small compound probes to exert biological efficacy at the systemic level, they must be able to reach their protein target inside the body, and thus they must possess a favorable pharmacologic profile. Recognized chemical properties associated with favorable pharmacologic attributes (Lipinski, C. A. et al., Adv Drug Deliv Rev 46, 3-26 (2001)) are provided in the figure, as are the associated chemical properties of KBU2046.

Female CD1 mice were dosed with 25 or 100 mg KBU2046/kg via oral gavage or intravenous injection (iv), and blood collected at 0 (pre-dose), 5, 10, 15, 30, 45, 60, 90, 120, 180, 240, 360, 480 and 960 minutes post dose. For each route and time point, N=3 mice were sampled. Mice were only sampled once. The results are shown in FIG. 12. Values are parameter estimates from a naïve pooled data approach in which single plasma concentrations measured for individual animals were pooled for both routes of administration of both doses and modeled simultaneously, as described herein. Note that estimates of parameter variability are not associated with this approach (Kataria, B. K. et al., Anesthesiology 80, 104-22 (1994)), and are thus not reported here.

For histologic examination of tissue, as shown in FIG. 14, cohorts of N=5 male 6-8 week old Balb/c athymic mice (Charles River Laboratories), which did not receive orthotopic implants, were treated with KBU2046 as described in FIG. 2a. After 35 days of treatment, the following organs were harvested at necropsy, and stained with H&E (alternative staining methods as indicated): heart, lungs, esophagus, stomach, colon, small intestine, liver (Trichrome staining), kidneys (Trichrome staining), adrenals, bladder, prostate, spleen, pancreas, brain, testes, and bone marrow and peripheral blood (Giemsa staining). Organs were examined for damage using a semi-quantitative histological scoring system, as described (Knodell, R. G. et al., Hepatology 1, 431-5 (1981)). No organ damage was observed, except in the livers of both control and treatment mice. Changes in the liver observed in control mice were not increased by KBU2046 treatment. Note that mice were immunocompromised, and that changes in the liver reflected episodic and minor foci of necrosis, consistent with a prior resolved infection; clinically, mice were all healthy. The graph above depicts the scoring of liver lesions, with each data point representing a single mouse.

For examination of organ function, studies used cohorts of 22-24 gm N=3 female and N=3 male (i.e., N=6 mice total per dose cohort) CD1 (ICR) mice (Charles River). Note that for 22-24 gm/mouse, this translated to 5-7 week old females and 4.5-5.5 week old males. Mice were dosed once intravenously with KBU2046 at 0 (vehicle), 15, 75 or 125 mg/kg-body weight. On day 8 and 14, important organs were weighed, and the following parameters measured in blood: cholesterol, triglycerides, alanine aminotransferase (ALT), aspartate aminotransferase (AST), total bilirubin, glucose, phosphorus, total protein, calcium, blood urea nitrogen (BUN), creatinine, albumin, Na, K, Cl, white blood cells (with differential), red blood cells, hemoglobin, platelets. No abnormal alterations in any of these parameters were observed, and there were no significant differences between treatment and control mice. Immediate death was observed for mice dosed with 150 mg/kg IV.

As shown in FIG. 17, PC3-M cells were pre-treated with 10 μM KBU2046, genistein or vehicle control for 3 days, then with ±TGFβ, as indicated. Resultant cell lysate, as well as lysate from tumors from mice treated with 150 mg/kg KBU2046 or control mice (from FIG. 2a), were then probed with the KinomeView® panel of antibodies by Western blot. In instances where KBU2046 was inhibiting protein phosphorylation in cells and in tumors, a repeat experiment of PC3-M cells was conducted (Experiment #2). In addition to including PC3-M cells, as in Experiment #1, Experiment #2 expanded to examine effects on PC3 cells.

FIG. 19 shows the results of identifying the 83 kDa band using a proteomic approach. Proteins bound in this manner were analyzed in replicates of N=2 using PhosphoScan™ technology. This identified 483 phosphopeptide assignments from 306 parent proteins, with a mean false discovery rate of 0.30% (estimated via Sorcerer search of composite human database of forward and reverse protein entries). Proteins were considered if the average values for treatment and control were each 3 times that of background. Further, it was required that each of the N=2 replicate values (used to calculate the average) to be >/=2.5 fold above background. Interest was focused on which protein decreased with KBU2046, and it was required that the decrease be >/=2.5 fold, compared to control. According to these parameters, there were 19 phospho-proteins whose expression decreased in cells treated with KBU2046, compared to control. These are depicted herein. Recognizing that each of these proteins may have value, it was elected to remain focused on the 83 kDa protein, and therefore considered proteins that were +/−5 kDa of this value. Heat shock protein 90 (HSP90)(3 met this criteria. FIG. 19b shows the evaluation of HSP90β levels. In order to assess whether phosphorylation changes detected in FIG. 17a were not due to changes in HSP90β protein, total HSP90β protein levels were measured by Western blot, after treatment of PC3 and PC3-M cells as described in FIG. 18a.

As shown in FIG. 16, proteins meeting the following criteria were sought, and did so at both 0.5 and 10 μM concentrations of KBU2046-biotin (in the absence of free KBU2046): Z-Score greater than 2.5, Z-Factor greater than 0.5, CI P-value less than 0.05, negative control value <2,000 (relative fluorescence units; RFUs), and a signal/negative control signal of >10 and >5 for 10 μM and 0.5 μM conditions, respectively. This yielded 3 proteins, shown in table, for an initial hit rate of 0.03%. Next, free KBU2046 was needed to inhibit binding by >75%. Of the two remaining candidates, isovaleryl-CoA dehydrogenase (ICD) was eliminated. Its binding signal doubled on going from 0.5 to 10, while percent competition by free KBU2046 markedly decreased; non-specific binding by the biotin-linker moiety was suspected. Cysteine and glycine-rich protein 1 (CSRP1) was deemed the top protein, though its binding characteristics at 10 μM+free KBU2046 were similar to ICD, albeit not as extreme. Thus, it was evaluated whether KBU2046-biotin would pull down recombinant CSRP1, and no evidence of such was observed.

There were two additional notable findings from protein array studies. First, the positive control used in protein arrays was staurosporine. Staurosporine is similar to genistein in that both are small compound natural products that are broad spectrum kinase inhibitors. In contrast to the lack of binding by KBU2046-biotin, staurosporine bound to 214 proteins at levels that were >/=10 fold above that of background. The vast majority of these proteins were kinases. Second, both HSP90β and CDC37 were present on the protein arrays, and were not bound by KBU2046-biotin.

The analysis began with experimentally determined structural information, including the crystal structures of human HSP90β (PDBs 1uym, 3nmq and 3pry) and HSP82-CDC37 complex from yeast (PDB 1us7), which were determined by X-ray diffraction-based crystallographic analysis. The HSP90β structure was experimentally probed using chemical cross-linking with mass spectrometry, employing chemical cross-linkers of various lengths, as previously described (Chavez, J. D., et al., Mol Cell Proteomics 12, 1451-67 (2013)). Cross-linked peptide samples were analyzed using ReACT (Weisbrod, C. R. et al., J Proteome Res (2013)) which allows targeted MS (Kataria, B. K. et al., Anesthesiology 80, 104-22 (1994)) to be carried out efficiently on each released peptide that satisfies expected PIR mass relationships (Tang, X. et al., Anal Chem 77, 311-8 (2005)). Further, the chemical structure of KBU2046 had been experimentally determined, as described herein. Finally, this experimental information was integrated using the APPLIED Pipeline (Analysis Pipeline for Protein Ligand Interactions and Experimental Determination) at the Argonne Leadership Computing Facility, Argonne National Laboratory, tuned for the 786,432 core BlueGene/Q Mira (Zhao, Y. et al., (Springer, 2007)), using a multi-stage pipeline that considers protein-protein/ligand interactions through evolutionary protein surface analysis (Binkowski, T. A. et al., J Mol Biol 332, 505-26 (2003); Binkowski, T. A. et. AL., BMC Struct Biol 8, 45 (2008); and Binkowski, T. A. ET AL., Protein Sci 14, 2972-81 (2005), robust homology modeling (Leaver-Fay, A. et al., Methods Enzymol 487, 545-74 (2011)), massively parallel docking simulations using mixed strategies (Lang, P. T. et al., RNA 15, 1219-30 (2009); Graves, A. P. et al., J Mol Biol 377, 914-34 (2008); Morris, G. M. et al., J Comput Chem 30, 2785-91 (2009); Deng, Y. et. al., J Chem Phys 128, 115103 (2008); and Wang, J. et. al., Biophys J 91, 2798-814 (2006)), and advanced, physics-based rescoring methodologies (Wang, J. et. al., Biophys J 91, 2798-814 (2006); Jiang, W. et. al, J Chem Theory Comput 5, 2583-2588 (2009); and Jiang, W. et. al., J Chem Theory Comput 6, 2559-2565 (2010)).

It was found that KBU2046 does not bind directly to HSP90β or CDC37 (FIG. 21), but that it does bind to the HSP90β-CDC37 complex (FIG. 5a). Therefore, the complex must afford a suitable binding pocket that is not independently present on either protein. Beginning with homology based modeling, existing structures from the protein data bank (PDB) were used. Structures of the human HSP90β N-terminal ATPase domain (PDB ids=1uym, 3nmq) and middle domain (PDB id=3pry) were used. The noncontiguous models cover 65% of the primary sequence, separated by a highly disordered region of 63 residues that terminates the ATP binding domain. No experimental models exist for the C-terminal region. The entirety of the HSP90β structure was then modeled against the HSP82 template from S. cerevisiae (PDB id=2cg9) (Leaver-Fay, A. et al., Methods Enzymol 487, 545-74 (2011)). A model of the complex of HSP90β-CDC37 was completed through superposition of the HSP90β model onto the structure of the S. cerevisiae HSP82-CDC37 complex (PDB id=1us7). The sequence identity between HSP90β and HSP82 is 94% at the CDC37 interface (86% for entire protein), thus, preserving the integrity of the interactions.

A marked feature of the HSP90 structure is the nucleotide binding site (see, FIG. 12). The site, with solvent accessible area of 496.2 Å (Shoemaker, R. H., Nat Rev Cancer 6, 813-23 (2006)) and volume of 301.3 Å (Kataria, B. K. et al., Anesthesiology 80, 104-22 (1994)); and (Binkowski, T. A. et al., Nucleic Acids Res 31, 3352-5 (2003)), has been well-characterized and targeted by a variety of compounds for anti-cancer activity. FIG. 23a shows the nucleotide binding site in yellow surface representation, bound with a purine-based inhibitor (PDB id=1uym) (Wright, L. et al., Chem Biol 11, 775-85 (2004)). When complexed with CDC37, an expansive surface, with solvent accessible area of 1446.4 Å (Shoemaker, R. H., Nat Rev Cancer 6, 813-23 (2006)) and volume of 2082.6 Å (Kataria, B. K. et al., Anesthesiology 80, 104-22 (1994)), is formed at the molecules' interface (FIG. 23b). Arg167_{cdc37} is drawn in to the nucleotide binding pocket and forms a hydrogen bond with the carboxyl side chain from Glu33_{HSP90} (Roe, S. M. et al., Cell 116, 87-98 (2004)). It was shown that Arg167_{cdc37} does not preclude access to the nucleotide binding site or displace any bound ligands (Roe, S. M. et al., Cell 116, 87-98 (2004)). It does, however, divide the large cleft into two distinct pockets: a newly formed pocket and the undisturbed, yet smaller, nucleotide binding site (FIG. 23c and FIG. 5b). The new pocket has solvent accessible area of 429.2 Å (Shoemaker, R. H., Nat Rev Cancer 6, 813-23 (2006)) and volume of 832.5 Å (Kataria, B. K. et al., Anesthesiology 80, 104-22 (1994)) and meets the criteria of a structural feature only present in the HSP90β-CDC37 complexed state.

The KBU2046 compound was docked into the newly formed pocket. A suite of docking software, representing different methodologies and approaches was applied. When allowed in the docking procedures, side chains from the HSP90β-CDC37 complex were allowed to be fully flexible. A consensus pose was reached with root mean square distance (RMSD) less than 1.1 Å over all atoms that exhibits no steric clashing with the complex. This model suggests that the molecule is capable of binding to this secondary site.

A dimer of the HSP90β structure in the closed conformation was modeled from S. cerevisiae HSP90A (PDB id=2cg9). In construction of the dimer, the HSP90β-CDC37 interface interactions were maintained. Position and orientation of the extended CDC37 regions were guided by cross-linking data that showed inter-domain cross-links between residues 53-347, 107-347, and 69-286 (FIG. 5d). This resulting structure shows agreement with other reported conformations (Vaughan, C. K. et al., Mol Cell 23, 697-707 (2006)). In this model, both the ATP and proposed KBU2046 pockets remain intact in the dimerized complex.

As shown in FIG. 24, HEK293T cells stably transfected with HSP90β-luciferase were transfected with 1 of 420 different FLAG-tagged protein kinases, treated with 10 μM KBU2046 for 3 days, and LUMIER assays performed as described (Taipale, M. et al., Cell 150, 987-1001 (2012)). Experiments had N=5 vehicle treated control plus N=5 KBU2046 treated separate wells of cells. Those yielding a t-test p value <0.05 in experiment #1, or those whose values were at or below baseline, were repeated in experiment #2. Those yielding a t-test p value <0.05 in both experiments and exhibiting a change in hetercomplex association with KBU2046 treatment in the same direction in both experiments were selected. Of 420 kinase proteins tested, 17 (4%) met these criteria (FIG. 6a). For these 17 kinases, the experiment was repeated except that HEK293T cells were treated with TGFβ for the last 24 hrs of the 3 day incubation (FIG. 6b).

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

1. A method for identifying one or more agents of interest that alters binding or activity of a client protein to a chaperone-co-chaperone complex, the method comprising:
a) forming a cell-free chaperone-co-chaperone complex in vitro with an isolated chaperone protein and a co-chaperone protein;
b) incubating the chaperone-co-chaperone complex with a client protein in the presence or absence of the one or more agents of interest;
c) assaying the binding of the client protein to the chaperone-co-chaperone complex or activity of the client protein in step (b); and
d) determining whether the one or more agents of interest alter the binding or activity of the client protein in step (c),
thereby identifying the one or more agents of interest that alter binding or activity of the client protein to the chaperone-co-chaperone complex.

2. The method of claim 1, further comprising incubating the isolated chaperone protein or the co-chaperone protein with the one or more agents of interest, wherein the one or more agents of interest do not bind to the isolated chaperone protein in the absence of the isolated co-chaperone protein or wherein the one or more agents of interest do not bind to the co-chaperone protein in the absence of the chaperone protein.

3. The method of claim 1, wherein the activity of the client protein to the chaperone-co-chaperone complex is kinase activity, E3 ligase activity or transcription factor activity or a combination thereof.

4. The method of claim 1, wherein in step (b), incubating conditions permit the client protein's activity, wherein the client protein's activity is kinase activity, E3 ligase activity and/or transcription factor activity.

5. The method of claim 4, further comprising determining phosphorylation status of the chaperone protein or the co-chaperone protein, wherein the phosphorylation status of the chaperone protein or the co-chaperone protein is altered by the one or more agents of interest.

6. (canceled)

7. The method of claim 1, wherein the chaperone protein is selected from the group consisting of Hsp100, Hsp104, Hsp110, Hsp90a, Hsp90b, Grp94, Grp78, Hsp72, Hsp71, Hsp70, Hsx70, Hsp60, Hsp47, Hsp40, Hsp27, Hsp20, hspb12, Hsp10, hspb7, Hspb6, Hspb4, HspB1, and alpha B crystallin.

8. The method of claim 1, wherein the co-chaperone protein is selected from the group consisting of Cdc37/p50, Aha1, auxilin, BAG1, CAIR-1/Bag-3, Chp1, Cyp40, Djp1, DnaJ, E3/E4-ubiquitin ligase, FKBP52, GAK, GroES, Hch1, Hip (Hsc70-interacting protein)/ST13, Hop (Hsp70/Hsp90 organizing protein)/STIP1, Mrj, PP5, Sacsin, SGT, Snl1, SODD/Bag-4, Swa2/Aux1, Tom34, Tom70, UNC-45, and WISp39.

9. The method of claim 1, wherein the chaperone-co-chaperone complex is selected from the group consisting of Hsp90b-Cdc37, or a chaperone-co-chaperone grouping from any of the chaperone protein of Hsp100, Hsp104, Hsp110, Hsp90a, Hsp90b, Grp94, Grp78, Hsp72, Hsp71, Hsp70, Hsx70, Hsp60, Hsp47, Hsp40, Hsp27, Hsp20, hspb12, Hsp10, hspb7, Hspb6, Hspb4, HspB1, and alpha B crystallin.

10. The method of claim 1, wherein the client protein is a kinase, a E3 ligase, a transcription factor, a polypeptide, MAP3K15, RJPK1, RAF1, NTRK1, MAP3K6, GSG2, RIPK2, NEK2, PRKCB1, LIMK1, TGFBR1, LOC340371, PRKACG, CAMK28, LOC81461, SGK3, NLK, or a fragment or derivative thereof.

11. (canceled)

12. The method of claim 1, wherein the one or more agents of interest alter cancer cell invasion and motility or inhibit cancer cell invasion and motility.

13. (canceled)

14. The method of claim 1, further comprising assaying one or more agents of interest for
a) cell migration, and identifying and selecting one or more one or more agents of interest as having reduced or no cell migration;
b) cytotoxicity, identifying and selecting one or more agents of interest having reduced or no cytotoxicity;
c) inhibiting cancer metastasis, and identifying and selecting one or more agents of interest that reduce or inhibit cancer metastasis;
d) promoting survival in a cancer xenograft animal model, and identifying and selecting one or more agents of interest promoting survival in the cancer xenograft animal model;
e) inhibiting organ destruction in an animal, and identifying and selecting one or more agents of interest having reduced or no organ destruction property;
f) altering phosphorylation of HSP90, and identifying and selecting one or more agents of interest altering phosphorylation of HSP90;
g) stabilizing the chaperone-co-chaperone complex, wherein the chaperone-co-chaperone complex is HSP90β/CDC37, and identifying and selecting one or more agents of interest stabilizing the chaperone-co-chaperone complex;
h) changes in signature of client proteins bound to the chaperone-co-chaperone complex, wherein the chaperone-co-chaperone complex is HSP90β/CDC37, and identifying and selecting one or more agents of interest changing the signature of client proteins bound to the chaperone-co-chaperone complex; or
i) altering post-translational modification of any chaperone, co-chaperone or client protein, wherein the post-translation modification is selected from the group consisting of phosphorylation, acetylation, nitrosylation, methylation, ubiquitination, sumoylation, acylation and oxidation.

15. (canceled)

16. (canceled)

17. (canceled)

18. (canceled)

19. (canceled)

20. (canceled)

21. (canceled)

22. The method of claim 14, wherein HSP90 is selected from the group consisting of HSP90β and HSP90β.

23. (canceled)

24. The method of claim 14, wherein altered phosphorylation of HSP90 comprises a decrease in phosphorylation of serine-226 of HSP90β.

25. (canceled)

26. (canceled)

27. The method of claim 14, wherein stabilizing HSP90β/CDC37 comprises stabilizing proteolytic degradation, preserving intact polypeptide or reducing proteolytic degradation products.

28. (canceled)

29. The method of claim 14, wherein the one or more agents of interest alters the signature of client proteins bound to HSP90β/CDC37 by reducing or inhibiting association of HSP90β/CDC37 to a subset of client proteins.

30. The method of claim 29, wherein the subset of client proteins are or comprise one or more kinases participating in cell motility, wherein the kinases participating in cell motility are selected from the group consisting of RAF1, RIPK1, SGK3, MAP3K15, NTRK1, MAP3K6, GSG2, RIPK2, NEK2, PRKCB1, LIMK1, TGFBR1, LOC340371, PRKACG, CAMK2B, LOC91461 and NLK.

31. (canceled)

32. (canceled)

33. The method of claim 1, wherein the one or more agents of interest are selected from the group consisting of small molecule, biological agent, peptide, polypeptide, antibody or derivative or fragment thereof, aptamer, PNA (peptide nucleic acid), nucleic acid, chemical compound, flavonoid, coumestan, prenylflavonoid, isoflavone, lignan and a substituted natural phenolic compound.

34. (canceled)

35. (canceled)

36. A method of treating cancer or metastatic cancer in a subject, the method comprising: identifying a subject in need of treatment; and administering a therapeutically effective amount of an agent of interest identified by the method of claim 1 or a salt or a derivative thereof.

37. A method of inhibiting or preventing cancer or metastatic cancer in a subject, the method comprising: identifying a subject in need of treatment; and administering a therapeutically effective amount of an agent of interest identified by the method of claim 1 or a salt or a derivative thereof.

38. (canceled)

39. (canceled)

40. A method for identifying one or more agents of interest that alters binding or activity of a client protein to a chaperone-co-chaperone complex, wherein the chaperone-co-chaperone complex is HSP90β/CDC37, the method comprising:
a) forming a cell-free chaperone-co-chaperone complex in vitro with an isolated chaperone protein and a co-chaperone protein;
b) incubating HSP90β/CDC37 with a client protein in the presence or absence of the one or more agents of interest;
c) assaying the binding of the client protein to HSP90β/CDC37 or activity of the client protein in step (b); and
d) determining whether the one or more agents of interest alter the binding or activity of the client protein in step (c),
thereby identifying the one or more agents of interest that alter binding or activity of the client protein to HSP90β/CDC37.

41. (canceled)

42. (canceled)
